(19) United States
(12) Patent Application Publication
(10) Pub. No.: US 2025/0213548 A1
(43) Pub. Date: Jul. 3, 2025

(54) PHARMACEUTICAL COMPOSITIONS FOR IMPROVING ORAL BIOAVAILABILITY

(71) Applicant: Bristol-Myers Squibb Company, Princeton, NJ (US)

(72) Inventors: Bo LANG, Somerset, NJ (US); David John GOOD, Westfield, NJ (US); Neil Raymond MATHIAS, North Brunswick, NJ (US); Divyakant S. DESAI, West Windsor, NJ (US); Monika LAVAN, Princeton, NJ (US); Freddy ARCE, Princeton, NJ (US)

(21) Appl. No.: 18/847,553

(22) PCT Filed: Mar. 17, 2023
(86) PCT No.: PCT/US2023/064656

§ 371 Date: Sep. 16, 2024

Related U.S. Application Data
(60) Provisional application No. 63/320,976, filed on Mar. 17, 2022.

(51) Int. Cl. A61K 31/455; A61K 9/00; A61K 31/24; A61K 38/12

(52) U.S. Cl. CPC A61K 31/455; A61K 9/0053; A61K 31/24; A61K 38/12

(57) ABSTRACT

In accordance with the present disclosure, pharmaceutical formulations that improve the oral bioavailability of biologically active compounds, including macrocyclic compounds, have been discovered.

### CROSS-REFERENCE TO RELATED APPLICATIONS

This application claims the priority benefit of U.S. Provisional Application No. 63/320,976, filed Mar. 17, 2022, which is incorporated herein by reference in its entirety.

### FIELD

The present disclosure provides pharmaceutical compositions which improve the oral bioavailability of biologically active compounds. Also disclosed are pharmaceutical compositions comprising macrocyclic compounds that bind to PD-L1 and are capable of inhibiting the interaction of PD-L1 with PD-1 and CD80.

### BACKGROUND

Despite an increasing trend in drug discovery favoring larger molecules such as millamolecules, poor oral bioavailability remains an impediment for more widespread use. Oral administration of large, hydrophilic molecules is a challenge due to pH and gastric/small intestinal enzyme interaction and low intestinal epithelial membrane permeability resulting from minimal passive or carrier-mediated transcellular permeation across phospholipid bilayers, as well as restricted paracellular transport through tight junctions. Other variables, such as plasma half-life and therapeutic index also impact the feasibility of the oral delivery of millamolecules.

In recent years, several cyclic peptides that block the interaction of PD-L1 with either PD-1 or CD80 have been reported (see, for example, U.S. Pat. Nos. 9,308,263; 9,850,283; 9,879,046; and 9,856,292). Such compounds are useful for enhancing, stimulating, and/or increasing an immune response in patients and can be used in the treatment of conditions such as septic shock and cancer. While oral delivery of these molecules would offer numerous advantages over delivery through injection, as in the case of other larger-sized molecules, developing oral formulations of such molecules has proven challenging. Thus there is a need for oral formulations that provide improved bioavailability of larger biologically active molecules such as macrocyclic peptides.

### SUMMARY

The present disclosure provides a pharmaceutical composition comprising a biologically active compound, a salcaprozate salt, and nicotinamide. In certain aspects, the salcaprozate salt is salcaprozate sodium (SNAC).

In some aspects, the composition further comprises one or more protease inhibitors. In some aspects, the one or more protease inhibitors comprises one or more trypsin inhibitors. In some aspects, the one or more trypsin inhibitors are isolated from bovine pancreas, raw avian egg white, soybean, or lima bean. In some aspects, the one or more protease inhibitors are selected from soybean trypsin inhibitor, aprotinin, lima bean trypsin inhibitor, ovomucoid trypsin inhibitor, and combinations thereof.

In some aspects, the biologically active compound comprises a cyclic peptide. In some aspects, the cyclic peptide comprises from 5 to 30 amino acids. In some aspects, the cyclic peptide comprises from 5 to 20 amino acids. In some aspects, the cyclic peptide comprises from 12 to 16 amino acids. In some aspects, the cyclic peptide is a compound of formula (I):

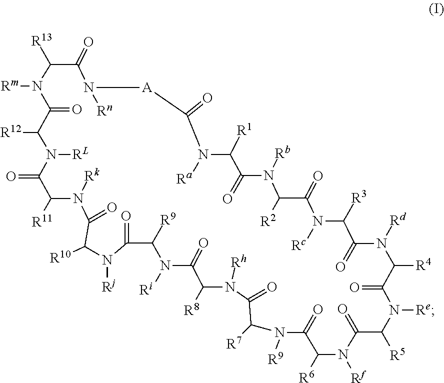

or a pharmaceutically acceptable salt thereof, wherein:
A is selected from:

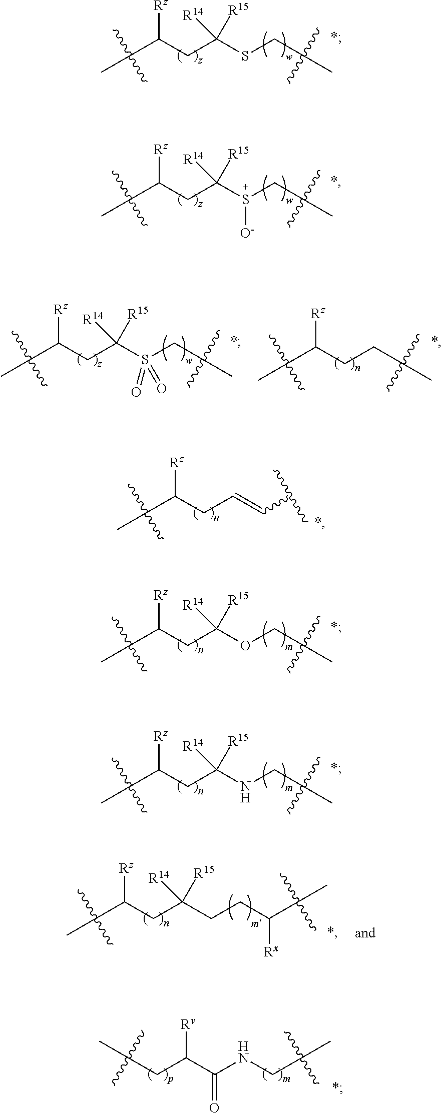

wherein

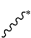

denotes the point of attachment to the carbonyl group and

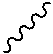

denotes the point of attachment to the nitrogen atom;
n is 0, 1, or 2;
m is 1 or 2;
m′ is 0 or 1;
z is 0, 1 or 2;
w is 1 or 2;
p is 0, 1, or 2;
R¹⁴ and R¹⁵ are independently selected from hydrogen and methyl;
R^{x} is selected from hydrogen, amino, hydroxy, and methyl;
R^{y} is hydrogen, methyl, or a natural amino acid side chain;
R^{z} is selected from hydrogen and —C(O)NHR¹⁶;
R¹⁶ is selected from hydrogen, —CHR¹⁷C(O)NH₂, —CHR¹⁷C(O)NHCHR¹⁷C(O)NH₂, —CH₂C(O)NHCH(R¹⁷)C(O)NHCH(R^{17a})C(O)NH₂; —CH₂C(O)NHCH(R¹⁷)CO₂H; and —(C(R^{17a})₂)₂—X′—R³⁰,
each R¹⁷ is independently selected from hydrogen, —CH₃, —CH₂OH, and —(CH₂)_{w}-triazolyl-X—R³⁵;
R³⁵ is selected from —CO₂H and CH₃;
each R^{17a} is independently selected from hydrogen and —CH₂CO₂H;
X′ is a chain of between 8 and 46 atoms wherein the atoms are selected from carbon and oxygen and wherein the chain may contain one, two, or three C(O)NH groups embedded therein; and wherein the chain is optionally substituted with one or two groups independently selected from —CO₂H, —C(O)NH₂, —CH₂C(O)NH₂, and —CH₂CO₂H;
R³⁰ is selected from —CO₂H, —C(O)NR^{w}R^{x}, and —CH₃ wherein R^{w} and R^{w′} are independently selected from hydrogen and C₁-C₆alkyl, provided that when X′ is all carbon, R³⁰ is other than —CH₃;
X is selected from
—(CH₂)₂CH(CO₂H)NHC(O)(CH₂);
—(CH₂CH₂O)g; and
—(CH₂CH₂O)_{g}CH₂CH₂NHC(O)CH₂CH₂CH(CO₂H)NHC(O)(CH₂)_{f};
f is 14, 15, or 16;
g is 3, 4, 5, 6, 7, 8, 9, 10, or 11;
R¹³ is selected from a natural amino acid, an unnatural amino acid, —(C(R^{17a})₂)₂—X′—R³⁰, and —(CH₂)_{w}-triazolyl-X—R³⁵,
R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{a}, R^{c}, R^{f}, R^{h}, Rⁱ, R^{j}, R^{m}, and Rⁿ are each independently selected from hydrogen and methyl;
R^{b} is hydrogen or methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R^{e} is hydrogen or methyl, or R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{k} is hydrogen or methyl, or, R^{k} and R¹¹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, and hydroxy; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects,
R^{c}, R^{f}, R^{h}, Rⁱ, R^{m}, and Rⁿ are hydrogen;
R^{b} is methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects,
R¹⁶ is —CH₂C(O)NHCH(R¹⁷)CO₂H or —(C(R^{17a})₂)₂—X′—R³⁰; and
R¹⁷ is —(CH₂)_{w}-triazolyl-X—R³⁵.

In some aspects, the cyclic peptide is a compound of formula (II):

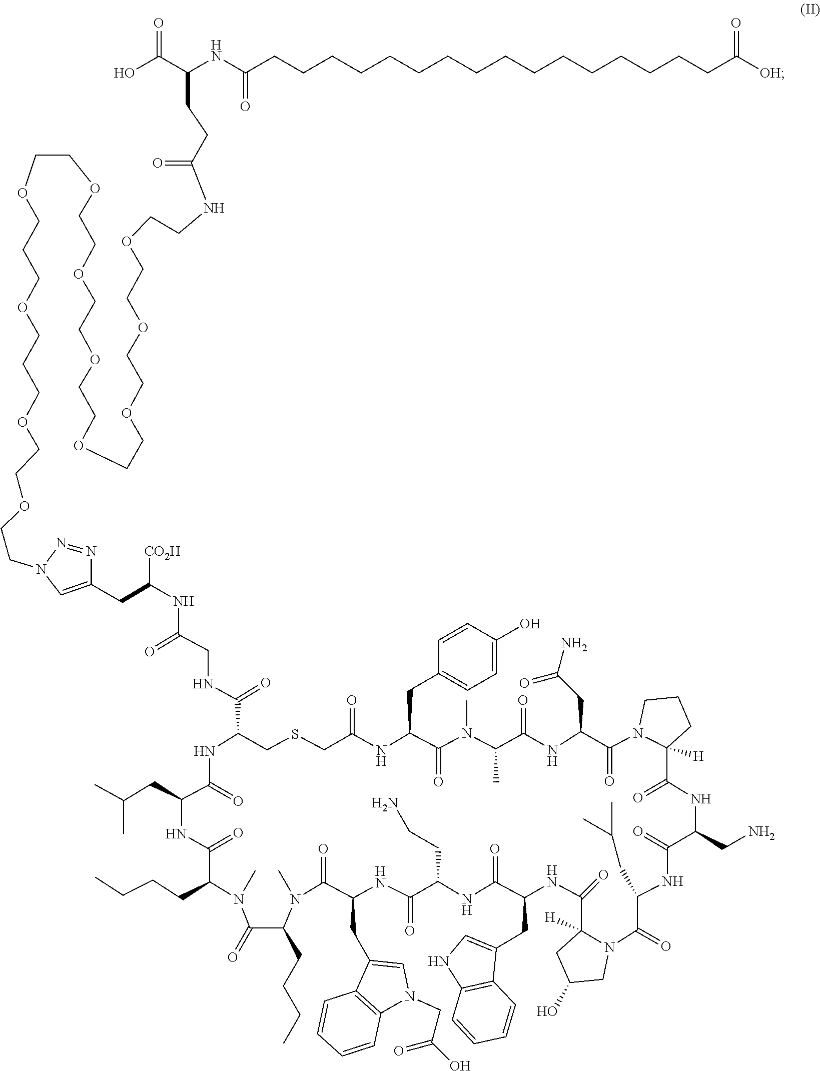 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

In some aspects, the biologically active compound is present in an amount of about 0.1% (w/w) to about 50% (w/w). In some aspects, the biologically active compound is present in an amount of about 1% (w/w) to about 45% (w/w). In some aspects, the biologically active compound is present in an amount of about 2% (w/w) to about 40% (w/w). In some aspects, the biologically active compound is present in an amount of about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 21% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), or about 45% (w/w).

In some aspects, the salcaprozate salt is present in an amount of about 30% (w/w) to about 95% (w/w). In some aspects, the salcaprozate salt is present in an amount of about 50% (w/w) to about 90% (w/w). In some aspects, the salcaprozate salt is present in an amount of about 60% (w/w) to about 85% (w/w). In some aspects, the salcaprozate salt is present in an amount of about 60% (w/w) to about 80% (w/w).

In some aspects, the biologically active compound and the salcaprozate salt are present in a w/w ratio of about 0.02 to about 1.5. In some aspects, the biologically active compound and the salcaprozate salt are present in a w/w ratio of about 0.03 to about 1.4. In some aspects, the biologically active compound and the salcaprozate salt are present in a w/w ratio of about 0.02, about 0.03, about 0.04, about 0.05, about 0.06, about 0.07, about 0.08, about 0.09, about 0.10, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, or about 1.5.

In some aspects, the nicotinamide is present in an amount of about 5% (w/w) to about 60% (w/w). In some aspects, the nicotinamide is present in an amount of about 10% (w/w) to about 50% (w/w). In some aspects, the nicotinamide is present in an amount of about 15% (w/w) to about 40% (w/w). In some aspects, the nicotinamide is present in an amount of about 20% (w/w) to about 30% (w/w).

In some aspects, the one or more protease inhibitors are present in an amount of about 0.1% (w/w) to about 50% (w/w), about 0.5% (w/w) to about 40% (w/w), about 0.75% (w/w) to about 30% (w/w), about 1% (w/w) to about 25% (w/w), or about 5% (w/w) to about 20% (w/w).

In some aspects, the present disclosure provides a pharmaceutical composition comprising:
(a) a cyclic peptide;
(b) salcaprozate sodium at a concentration of about 45% (w/w) to about 80% (w/w); and
(c) nicotinamide at a concentration of about 15% (w/w) to about 30% (w/w).

In some aspects, the present disclosure provides a pharmaceutical composition comprising:
(a) a cyclic peptide;
(b) salcaprozate sodium at a concentration of about 45% (w/w) to about 80% (w/w);
(c) nicotinamide at a concentration of about 15% (w/w) to about 30% (w/w); and
(d) a protease inhibitor at a concentration of about 1% (w/w) and about 20% (w/w).

In some aspects, the cyclic peptide is a compound of formula (I):

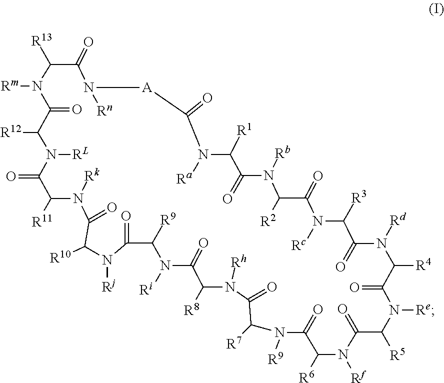

or a pharmaceutically acceptable salt thereof, wherein:
A is selected from:

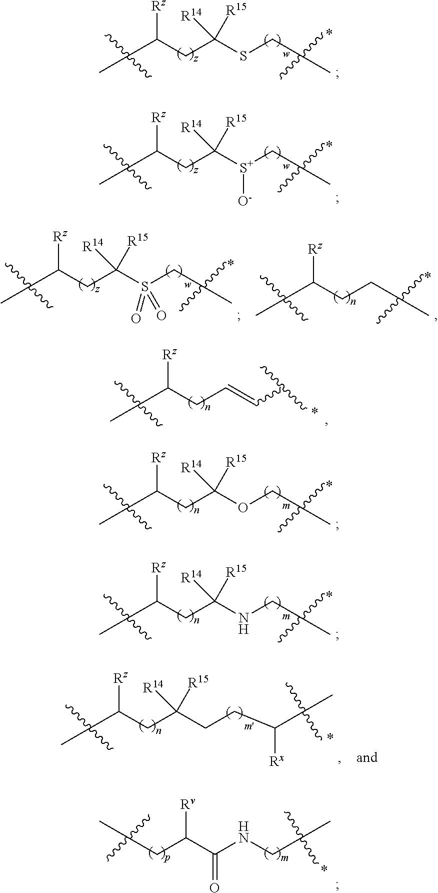

wherein

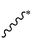

denotes the point of attachment to the carbonyl group and

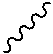

denotes the point of attachment to the nitrogen atom;
n is 0, 1, or 2;
m is 1 or 2;
m′ is 0 or 1;
z is 0, 1 or 2;
w is 1 or 2;
p is 0, 1, or 2;
R¹⁴ and R¹⁵ are independently selected from hydrogen and methyl;
R^{x} is selected from hydrogen, amino, hydroxy, and methyl;
R^{y} is hydrogen, methyl, or a natural amino acid side chain;
R^{z} is selected from hydrogen and —C(O)NHR¹⁶;
R¹⁶ is selected from hydrogen, —CHR¹⁷C(O)NH₂, —CHR¹⁷C(O)NHCHR¹⁷C(O)NH₂, —CH₂C(O)NHCH(R¹⁷)C(O)NHCH(R^{17a})C(O)NH₂; —CH₂C(O)NHCH(R¹⁷)CO₂H; and —(C(R^{17a})₂)₂—X′—R³⁰;
each R¹⁷ is independently selected from hydrogen, —CH₃, —CH₂OH, and —(CH₂)_{w}-triazolyl-X—R³⁵;
R³⁵ is selected from —CO₂H and CH₃;
each R^{17a} is independently selected from hydrogen and —CH₂CO₂H;
X′ is a chain of between 8 and 46 atoms wherein the atoms are selected from carbon and oxygen and wherein the chain may contain one, two, or three C(O)NH groups embedded therein; and wherein the chain is optionally substituted with one or two groups independently selected from —CO₂H, —C(O)NH₂, —CH₂C(O)NH₂, and —CH₂CO₂H;
R³⁰ is selected from —CO₂H, —C(O)NR^{w}R^{x}, and —CH₃ wherein R^{w} and R^{w′} are independently selected from hydrogen and C₁-C₆alkyl, provided that when X′ is all carbon, R³⁰ is other than —CH₃;
X is selected from
—(CH₂)₂CH(CO₂H)NHC(O)(CH₂)_{f};
—(CH₂CH₂O)_{g}; and
—(CH₂CH₂O)_{g}CH₂CH₂NHC(O)CH₂CH₂CH(CO₂H)NHC(O)(CH₂)_{f};
f is 14, 15, or 16;
g is 3, 4, 5, 6, 7, 8, 9, 10, or 11;
R¹³ is selected from a natural amino acid, an unnatural amino acid, —(C(R^{17a})₂)₂—X′—R³⁰, and —(CH₂)_{w}-triazolyl-X—R³⁵;

R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{a}, R^{c}, R^{f}, R^{h}, Rⁱ, R^{j}, R^{m}, and Rⁿ are each independently selected from hydrogen and methyl;
R^{b} is hydrogen or methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R^{e} is hydrogen or methyl, or R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{k} is hydrogen or methyl, or, R^{k} and R¹¹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, and hydroxy; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects,
R^{c}, R^{f}, R^{h}, Rⁱ, R^{m}, and Rⁿ are hydrogen;
R^{b} is methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects,
R¹⁶ is —CH₂C(O)NHCH(R¹⁷)CO₂H or —(C(R^{17a})₂)₂—X′—R³⁰; and
R¹⁷ is —(CH₂)_{w}-triazolyl-X—R³⁵.

In some aspects, the cyclic peptide is a compound of formula (II):

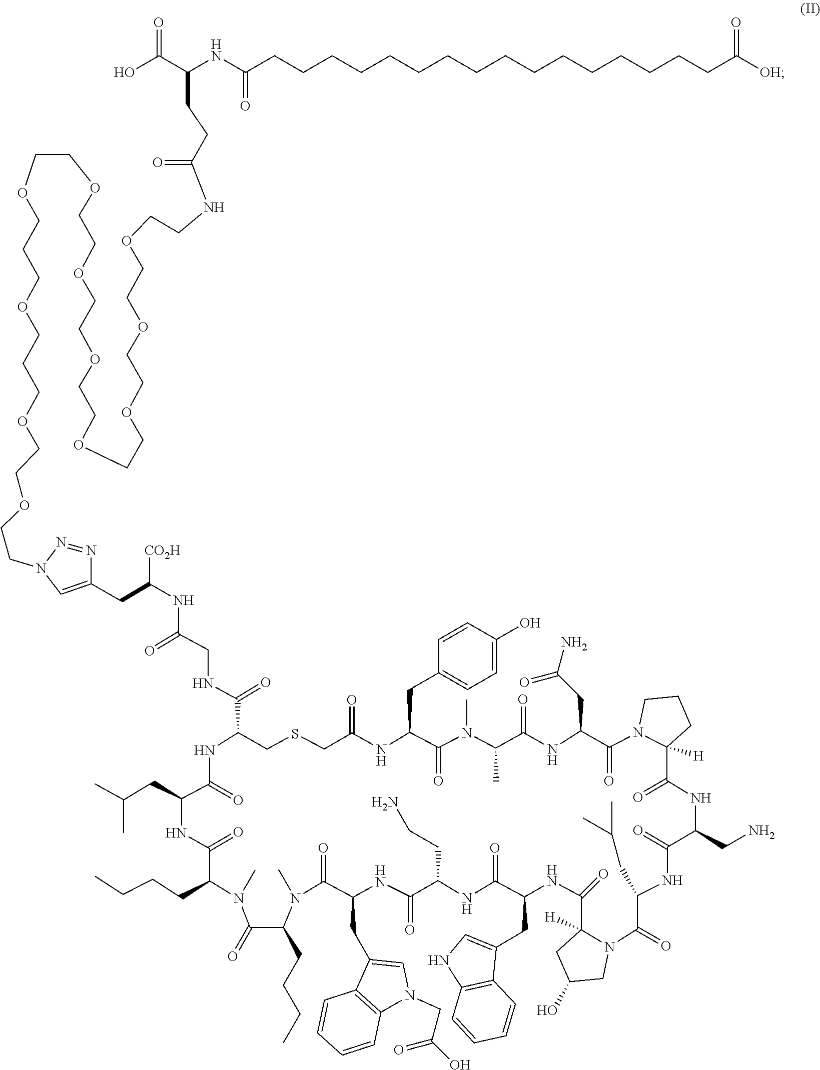 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

In some aspects, the cyclic peptide is present in an amount of about 1% (w/w) to about 40% (w/w).

In some aspects, the present disclosure provides a kit or article of manufacture comprising (i) the pharmaceutical composition of any one of the above aspects, and (ii) instructions for use.

In some aspects, the present disclosure provides a method of improving oral bioavailability of a biologically active compound in a subject in need thereof comprising formulating the biologically active compound with a salcaprozate salt and nicotinamide. In some aspects of the method, the formulated biologically active compound has improved oral bioavailability compared to the biologically active compound without the salcaprozate salt and nicotinamide. In some aspects of the method, the oral bioavailability is improved at least by about 10%, at least by about 20%, at least by about 30%, at least by about 40%, at least by about 50%, at least by about 60%, at least by about 70%, at least by about 80%, at least by about 90%, at least about 100%, at least about 200%, at least about 300% at least about 400%, at least about 500%, at least about 600%, at least 700%, or at least 800%. In some aspects of the method, the salcaprozate salt is salcaprozate sodium.

In some aspects of the method, the biologically active compound is a cyclic peptide. In some aspects of the method, the cyclic peptide comprises from 5 to 30 amino acids. In some aspects, the cyclic peptide comprises from 5 to 20 amino acids. In some aspects of the method, the cyclic peptide comprises from 12 to 16 amino acids. In some aspects of the method, the cyclic peptide is a compound of formula (I):

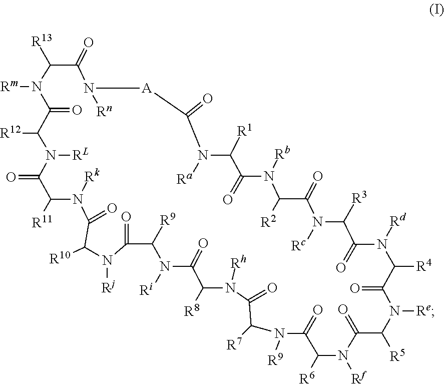

or a pharmaceutically acceptable salt thereof, wherein:
A is selected from:

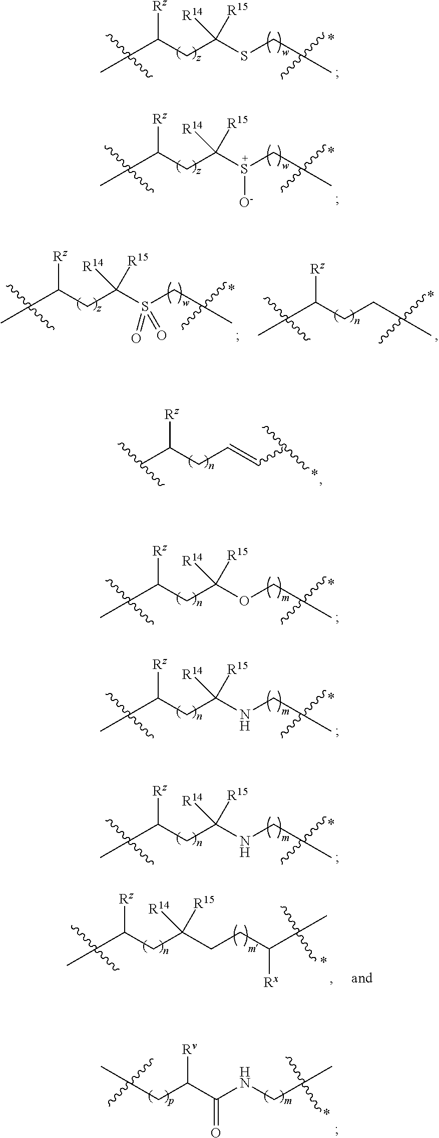

wherein

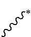

denotes the point of attachment to the carbonyl group and

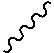

denotes the point of attachment to the nitrogen atom;
n is 0, 1, or 2;
m is 1 or 2;
m′ is 0 or 1;
z is 0, 1 or 2;
w is 1 or 2;
p is 0, 1, or 2;
R¹⁴ and R¹⁵ are independently selected from hydrogen and methyl;
R^{x} is selected from hydrogen, amino, hydroxy, and methyl;
R^{y} is hydrogen, methyl, or a natural amino acid side chain;
R^{z} is selected from hydrogen and —C(O)NHR¹⁶;
R¹⁶ is selected from hydrogen, —CHR¹⁷C(O)NH₂, —CHR¹⁷C(O)NHCHR¹⁷C(O)NH₂, —CH₂C(O)NHCH(R¹⁷)C(O)NHCH(R^{17a})C(O)NH₂; —CH₂C(O)NHCH(R¹⁷)CO₂H; and —(C(R^{17a})₂)₂—X′—R³⁰;
each R¹⁷ is independently selected from hydrogen, —CH₃, —CH₂OH, and —(CH₂)_{w}-triazolyl-X—R³⁵;
R³⁵ is selected from —CO₂H and CH₃;
each R^{17a} is independently selected from hydrogen and —CH₂CO₂H;
X′ is a chain of between 8 and 46 atoms wherein the atoms are selected from carbon and oxygen and wherein the chain may contain one, two, or three C(O)NH groups embedded therein; and wherein the chain is optionally substituted with one or two groups independently selected from —CO₂H, —C(O)NH₂, —CH₂C(O)NH₂, and —CH₂CO₂H;
R³⁰ is selected from —CO₂H, —C(O)NR^{w}R^{x}, and —CH₃ wherein R^{w} and R^{w′} are independently selected from hydrogen and C₁-C₆alkyl, provided that when X′ is all carbon, R³⁰ is other than —CH₃;
X is selected from
—(CH₂)₂CH(CO₂H)NHC(O)(CH₂)_{f};
—(CH₂CH₂O)_{g}; and
—(CH₂CH₂O)_{g}CH₂CH₂NHC(O)CH₂CH₂CH(CO₂H)NHC(O)(CH₂)_{f};
f is 14, 15, or 16;
g is 3, 4, 5, 6, 7, 8, 9, 10, or 11;
R¹³ is selected from a natural amino acid, an unnatural amino acid, —(C(R^{17a})₂)₂—X′—R³⁰, and —(CH₂)_{w}-triazolyl-X—R³⁵;
R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{a}, R^{c}, R^{f}, R^{h}, Rⁱ, R^{j}, R^{m}, and Rⁿ are each independently selected from hydrogen and methyl;
R^{b} is hydrogen or methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R^{e} is hydrogen or methyl, or R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{k} is hydrogen or methyl, or, R^{k} and R¹¹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, and hydroxy; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects of the method,
R^{c}, R^{f}, R^{h}, Rⁱ, R^{m}, and Rⁿ are hydrogen;
R^{b} is methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

In some aspects of the method,
R¹⁶ is —CH₂C(O)NHCH(R¹⁷)CO₂H or —(C(R^{17a})₂)₂—X′—R³⁰; and
R¹⁷ is —(CH₂)_{w}-triazolyl-X—R³⁵.

In some aspects of the method, the cyclic peptide is a compound of formula (II):

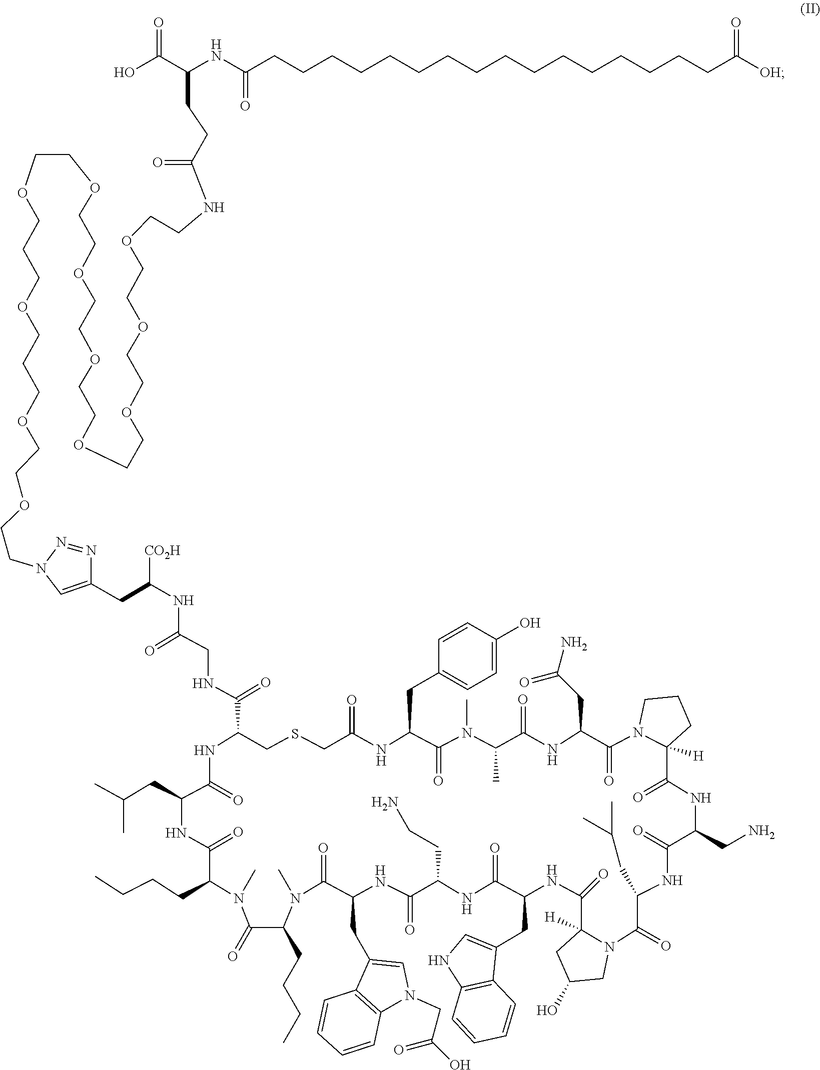 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

In some aspects, the present disclosure provides a composition comprising a compound of formula (II):

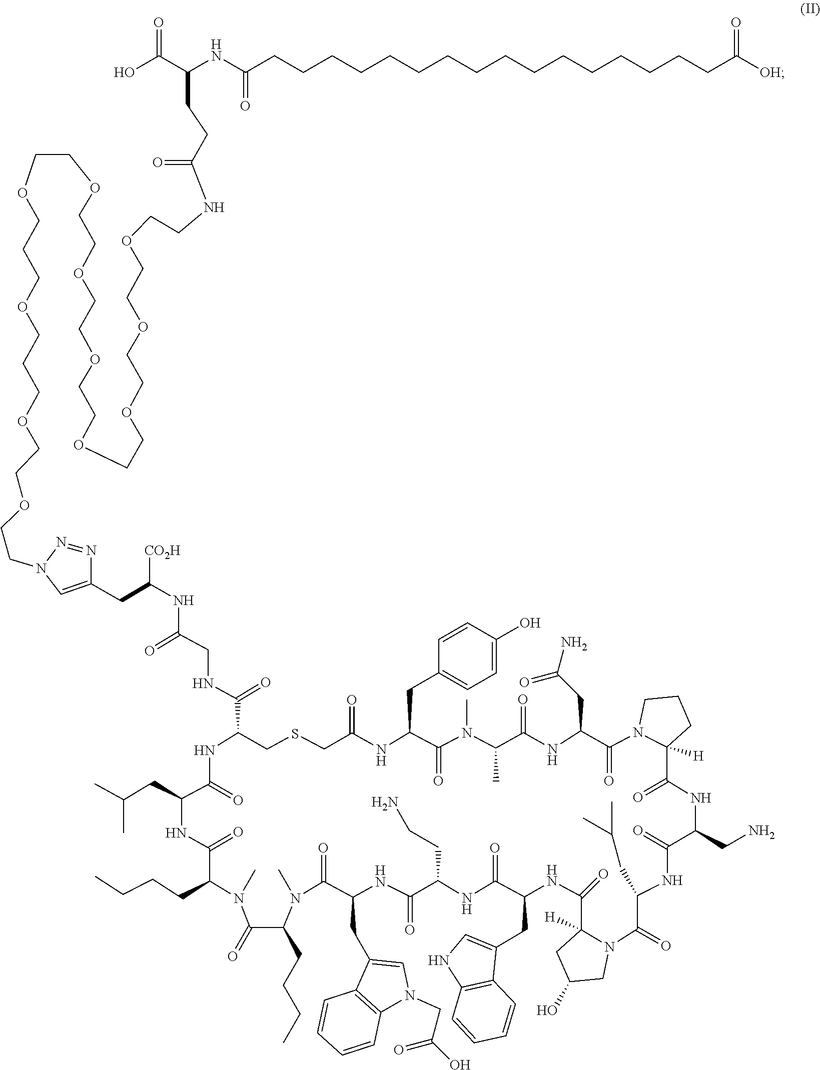 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof;

salcaprozate sodium, and nicotinamide, wherein the salcaprozate sodium and nicotinamide provide an oral bioavailability of >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

In some aspects, the present disclosure provides a method of improving the oral bioavailability of a compound of formula (II):

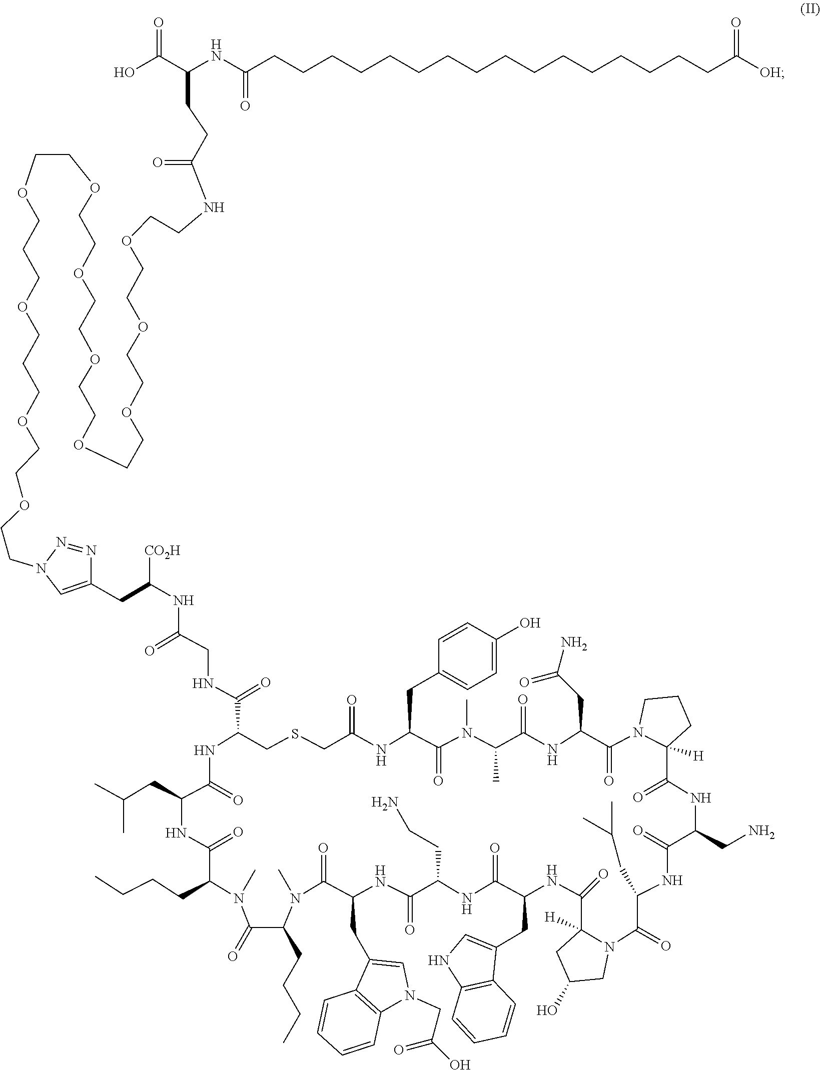 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof, to >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%, the method comprising formulating the biologically active compound with a salcaprozate salt and nicotinamide.

In some aspects, the present disclosure provides a method of inhibiting growth, proliferation, or metastasis of cancer cells in a subject in need thereof, said method comprising administering to the subject a therapeutically effective amount of a pharmaceutical composition, or kit of any of the above aspects. In some aspects of the method, the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and a hematological malignancy.

In some aspects, the present disclosure provides a method of enhancing, stimulating, and/or increasing an immune response in a subject in need thereof, said method comprising administering to the subject a therapeutically effective amount of a pharmaceutical composition, or kit of any of the above aspects.

In some aspects, the present disclosure provides a method of treating septic shock in a subject in need thereof, the method comprising administering to the subject a therapeutically effective amount of a pharmaceutical composition, or kit of any of the above aspects.

In some aspects, the present disclosure provides a method of blocking the interaction of PD-L1 with PD-1 and/or CD80 in a subject, said method comprising administering to the subject a therapeutically effective amount of a pharmaceutical composition, or kit of any of the above aspects.

In some aspects of the methods, the formulation is administered orally. In some aspects of the method, the formulation is intravenously administered. In some aspects of the methods, the formulation is intraduodenally administered.

In some aspects, the present disclosure provides a method of administering a compound of formula (II):

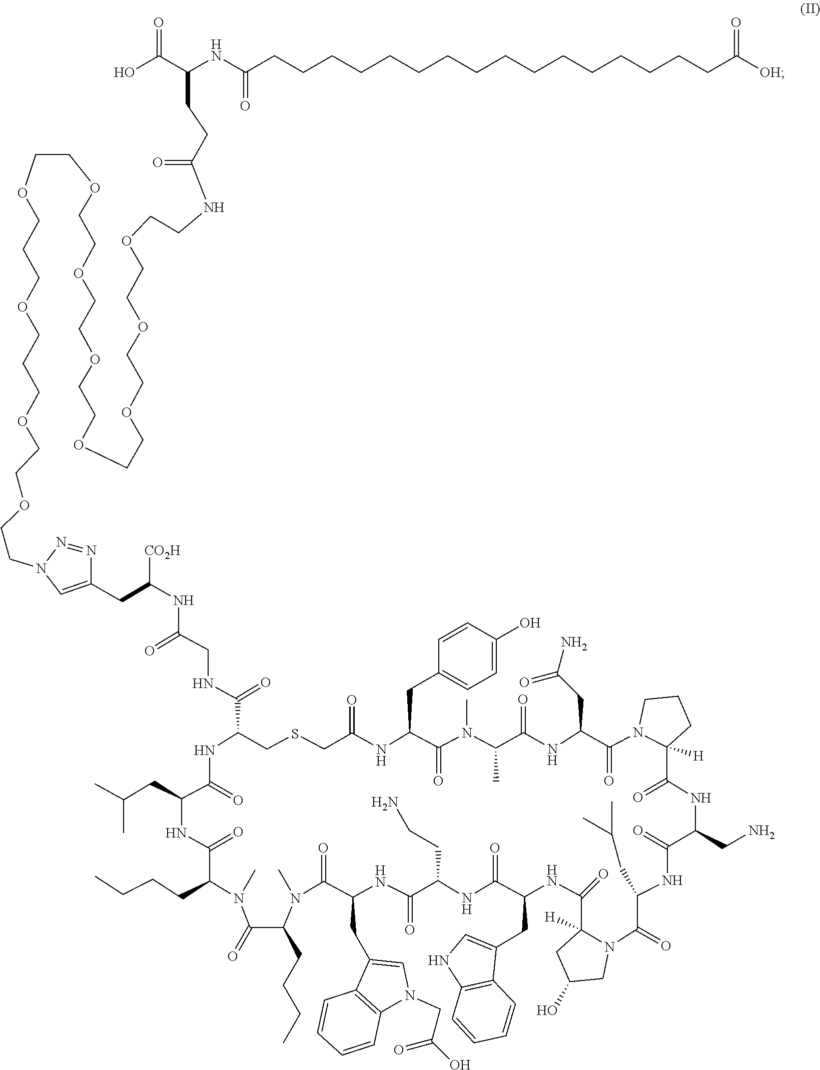 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

comprising administering the compound orally with salcaprozate sodium and nicotinamide wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

In some aspects the present disclosure provides an orally adminstered composition comprising a compound of formula (II):

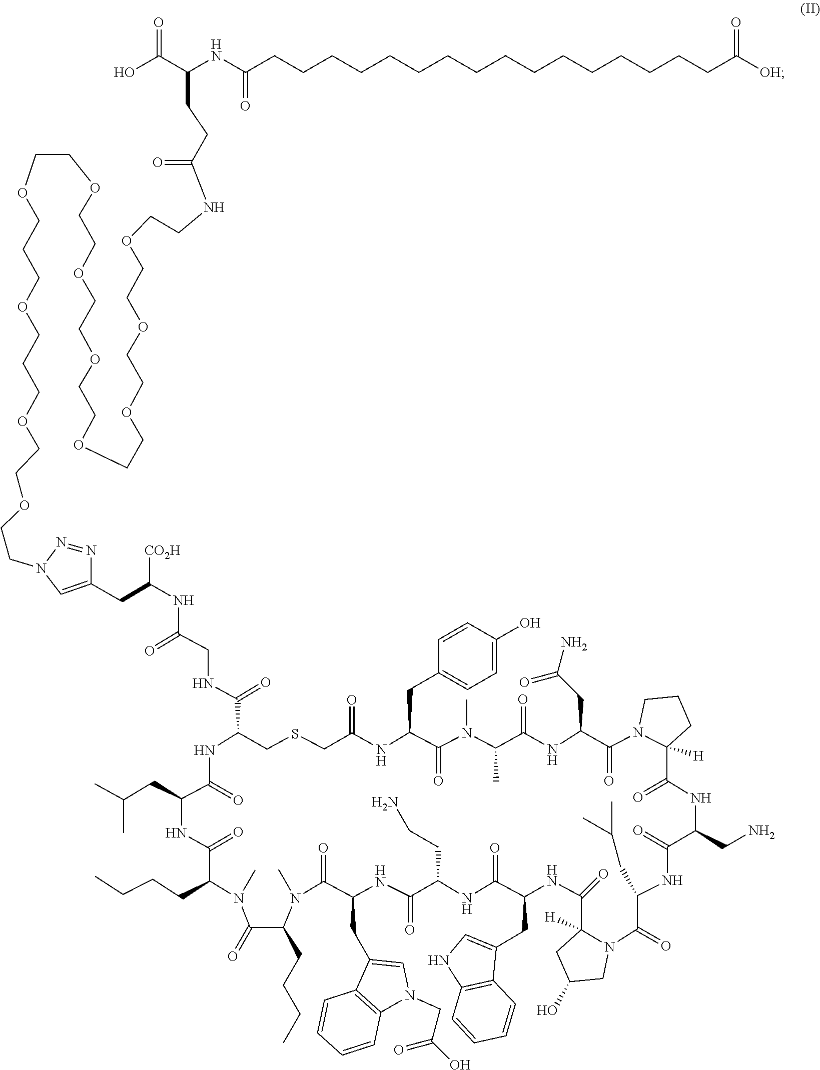 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof,
salcaprozate sodium, and nicotinamide, wherein the oral bioavailability of the compound of formula (II) is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

In some aspects, the present disclosure provides a method of administering a compound of formula (II):

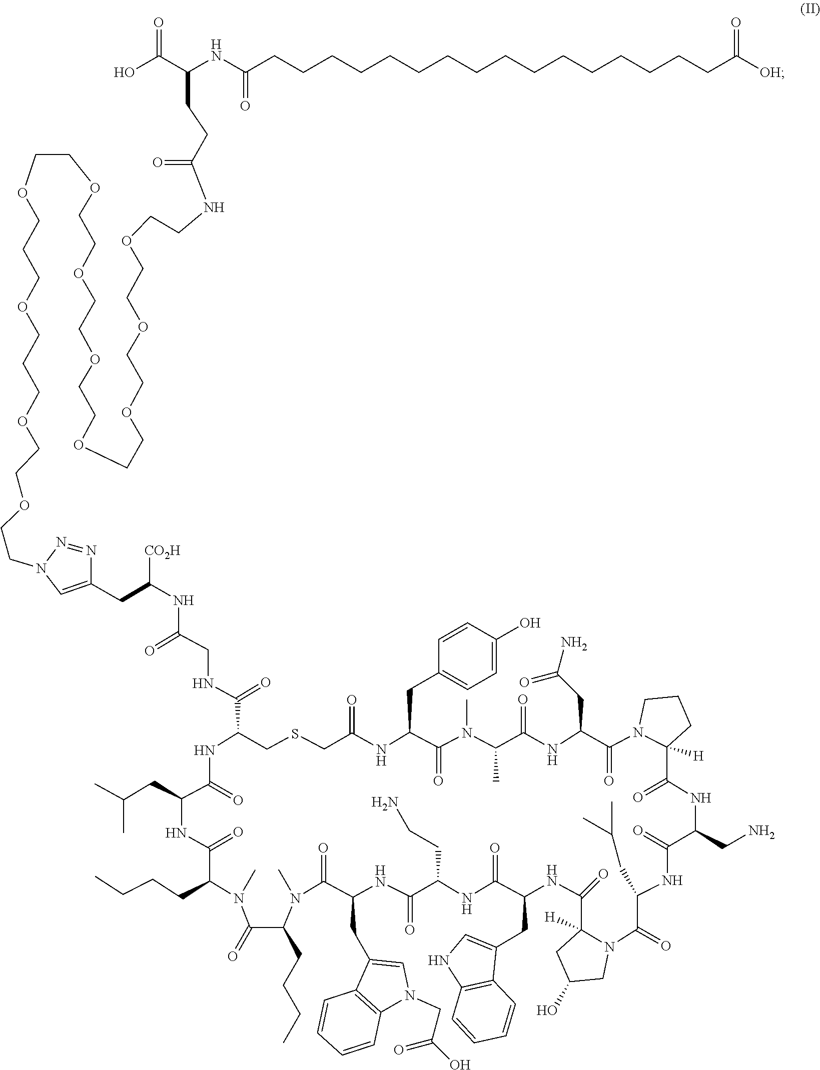 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

comprising administering the compound orally with salcaprozate sodium and nicotinamide wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

In some aspects, the present disclosure provides a method of orally administering a peptide comprising orally administering a compound of formula (II):

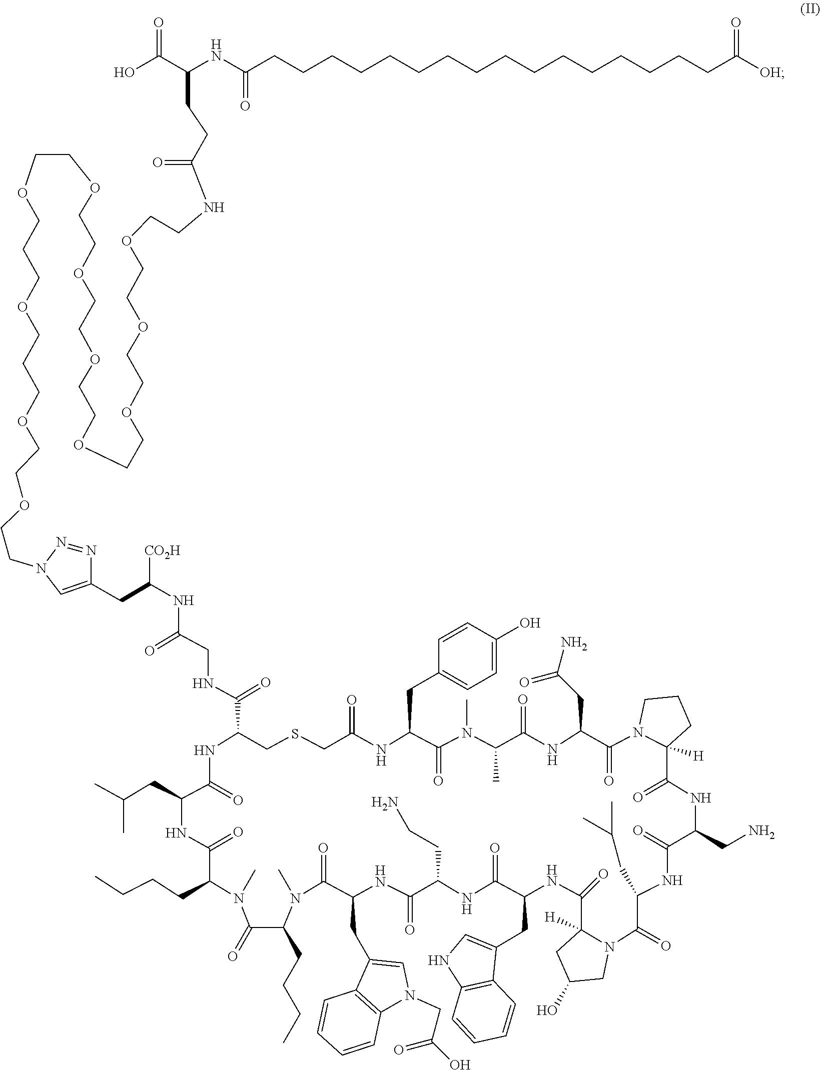 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof,
salcaprozate sodium, and nicotinamide, wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

### BRIEF DESCRIPTION OF THE FIGURES

FIG. 1 shows the gastric and intestinal absorption in rat of Formulations A and B.

FIG. 2 shows the pharmacokinetic profile in dog of tablet formulations of Compound (II) containing differing amounts of salcaprozate sodium (SNAC) in comparison to a tablet formulation of Compound (II) containing SNAC and a protease inhibitor (PI).

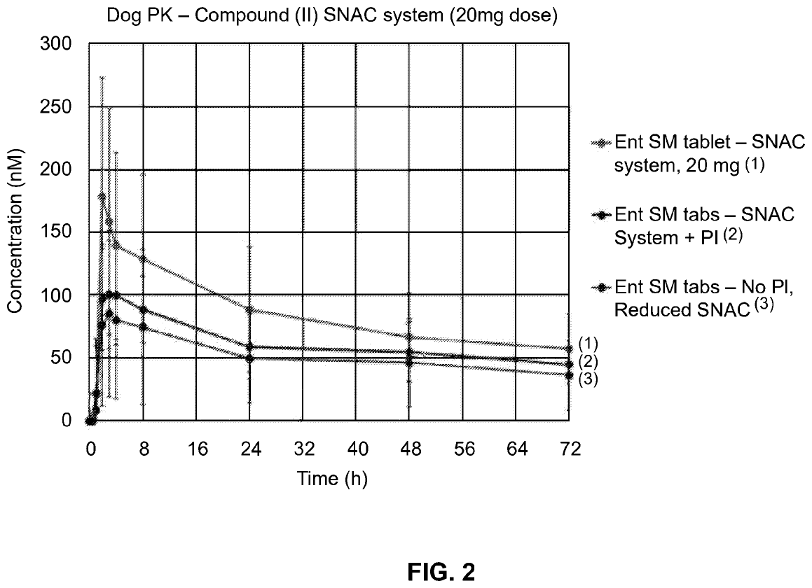

FIG. 3 shows the pharmacokinetic profile in dog of tablet formulations containing different amounts of Compound (II).

FIG. 4 shows the pharmacokinetic profile in dog of formulated tablets with containing higher or lower amounts of Compound (II).

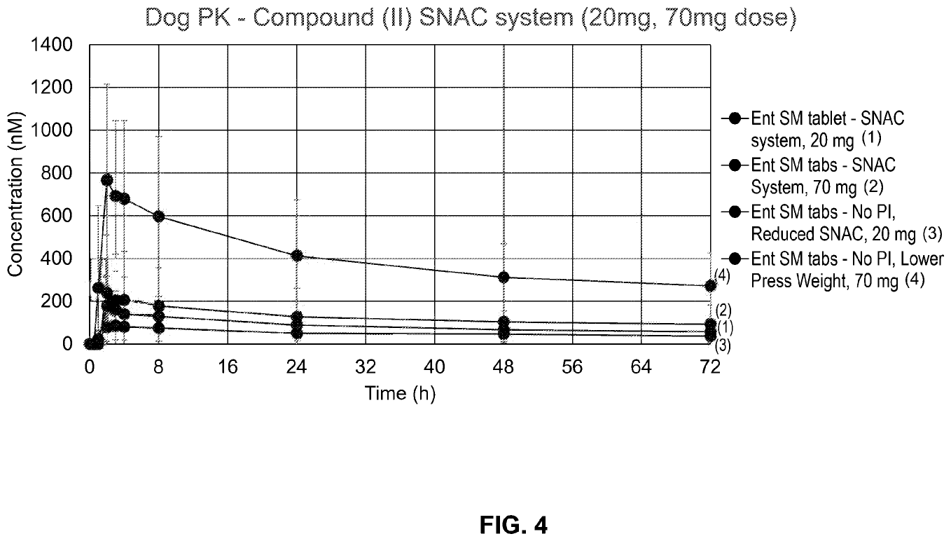

FIG. 5A shows the concentration of Compound (II) in individual dogs dosed with a formulation prepared using higher press weight (700 lb compression force).

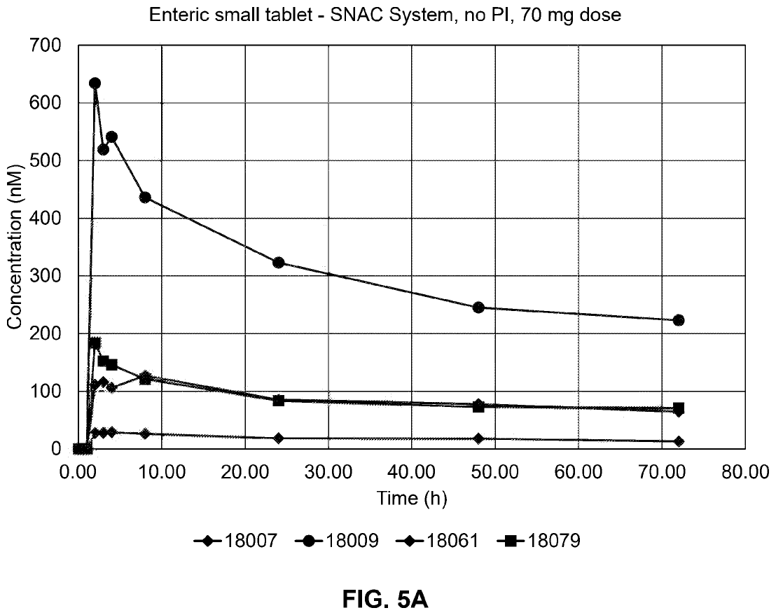

FIG. 5B shows the pharmacokinetic profile in individual dogs of a tablet formulation of Compound (II) prepared using lower press weight (500 lb compression force).

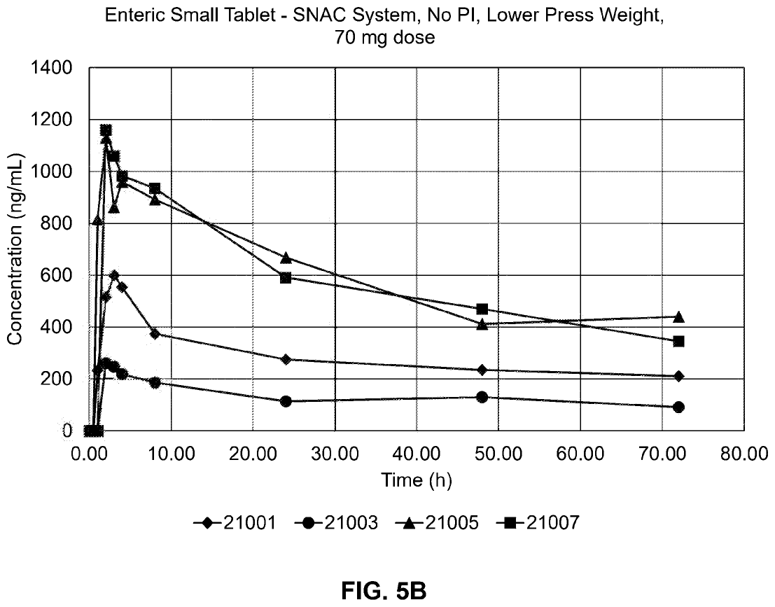

FIG. 6 shows the exposure of Compound (II) in dogs dosed with 50 mg and 100 mg dosages of Compound (II).

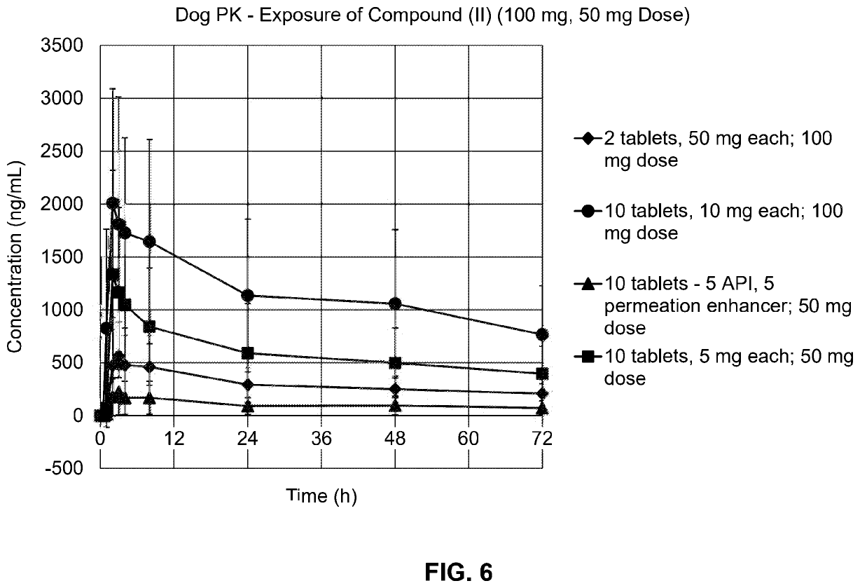

FIG. 7 shows the exposure of Compound (II) in cyno monkeys dosed with 12 mg dosage of Compound (II).

FIG. 8 shows the exposure of Compound (II) in dogs dosed with 70 mg dosages of Compound (II).

FIG. 9 shows the exposure of Compound (II) in dogs dosed with 50 mg dosages of Compound (II).

FIG. 10 shows the exposure of Compound (II) in dogs dosed with 100 mg dosages of Compound (II).

### DETAILED DESCRIPTION

The present disclosure is directed toward a pharmaceutical composition comprising a biologically active compound; a salcaprozate salt (such as salcaprozate sodium), nicotinamide, and optionally one or more protease inhibitors. The composition can improve the bioavailability of the biologically active compound.

### I. Definitions

In order that the present description can be more readily understood, certain terms are first defined. Additional definitions are set forth throughout the detailed description.

Unless otherwise indicated, any atom with unsatisfied valences is assumed to have hydrogen atoms sufficient to satisfy the valences.

The singular forms “a,” “an,” and “the” include plural referents unless the context dictates otherwise. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements or use of a negative limitation.

The term “or” is a logical disjunction (i.e., and/or) and does not indicate an exclusive disjunction unless expressly indicated such as with the terms “either,” “unless,” “alternatively,” and words of similar effect.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Those of ordinary skill in the art are aware that an amino acid includes a compound represented by the general structure:

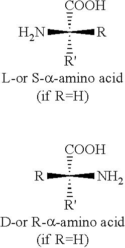

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which produce the desired activity. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

The terms “natural amino acid side chain” and “naturally occurring amino acid side chain”, as used herein, refer to side chain of any of the naturally occurring amino acids (i.e., alanine, arginine, asparagine, aspartic acid, cysteine, glutamine, glutamic acid, glycine, -histidine, isoleucine, leucine, lysine, methionine, phenylalanine, proline, serine, threonine, tryptophan, tyrosine, and valine) usually in the S-configuration (i.e., the L-amino acid).

The terms “unnatural amino acid side chain” and “non-naturally occurring amino acid side chain”, as used herein, refer to a side chain of any naturally occurring amino acid usually in the R-configuration (i.e., the D-amino acid) or to a group other than a naturally occurring amino acid side chain in R- or S-configuration (i.e., the D- or L-amino acid, respectively) selected from:
C₂-C₇alkenyl, C₁-C₃alkoxyC₁-C₃alkyl, C₁-C₆alkoxycarbonylC₁-C₃alkyl, C₁-C₇alkyl, C₁-C₃alkylsulfanylC₁-C₃alkyl, amidoC₁-C₃alkyl, aminoC₁-C₃alkyl, azaindolylC₁-C₃alkyl, benzothiazolylC₁-C₃alkyl, benzothienylC₁-C₃alkyl, benzyloxyC₁-C₃alkyl, carboxyC₁-C₃alkyl, C₃-C₁₄cycloalkylC₁-C₃alkyl, diphenylmethyl, furanylC₁-C₃alkyl, imidazolylC₁-C₃alkyl, naphthylC₁-C₃alkyl, pyridinylC₁-C₃alkyl, thiazolylC₁-C₃alkyl, thienylC₁-C₃alkyl;
biphenylC₁-C₃alkyl wherein the biphenyl is optionally substituted with a methyl group;
heterocyclyl optionally substituted with one, two, three, four, or five groups independently selected from C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂;
indolylC₁-C₃alkyl, wherein the indolyl part is optionally substituted with one group selected from C₁-C₃alkoxycarbonylC₁-C₃alkyl, C₁-C₃alkyl, carboxyC₁-C₃alkyl, halo, hydroxy, and phenyl, wherein the phenyl is further optionally substituted by one, two, or three groups independently selected from C₁-C₃alkoxy, C₁-C₃alkyl, and halo;
NR^{x}R^{y}(C₁-C₇alkyl), wherein R^{x} and R^{y} are independently selected from hydrogen, C₂-C₄alkenyloxycarbonyl, C₁-C₃alkyl, C₁-C₃alkylcarbonyl, C₃-C₁₄cycloalkylcarbonyl, furanylcarbonyl, and phenylcarbonyl. When the alkyl linker contains more than one carbon, an additional NR^{x}R^{y} group can be on the chain.
NR^{t}R^{u}carbonylC₁-C₃alkyl, wherein R^{t} and R^{u} are independently selected from hydrogen, C₁-C₃alkyl, and triphenylmethyl;
phenyl optionally substituted with one, two, three, four, or five groups independently selected from C₁-C₄alkoxy, C₁-C₄alkyl, C₁-C₃alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂;
phenylC₁-C₃alkyl wherein the phenyl part is optionally substituted with one, two, three, four, or five groups independently selected from C₁-C₄alkoxy, C₁-C₄alkyl, C₁-C₇alkylsulfonylamino, amido, amino, aminoC₁-C₃alkyl, aminosulfonyl, carboxy, cyano, halo, haloC₁-C₃alkyl, hydroxy, —NC(NH₂)₂, nitro, and —OP(O)(OH)₂; and
phenoxyC₁-C₃alkyl wherein the phenyl is optionally substituted with a C₁-C₃alkyl group.

The term “C₂-C₄alkenyl”, as used herein, refers to a straight or branched chain group of two to four carbon atoms containing at least one carbon-carbon double bond.

The term “C₂-C₇alkenyl”, as used herein, refers to a straight or branched chain group of two to seven carbon atoms containing at least one carbon-carbon double bond.

The term “C₂-C₄alkenyloxy”, as used herein, refers to a C₂-C₄alkenyl group attached to the parent molecular moiety through an oxygen atom.

The term “C₂-C₄alkenyloxycarbonyl”, as used herein, refers to a C₂-C₄alkenyloxy group attached to the parent molecular moiety through a carbonyl group.

The term “C₁-C₃alkoxy”, as used herein, refers to a C₁-C₃alkyl group attached to the parent molecular moiety through an oxygen atom.

The term “C₁-C₄alkoxy”, as used herein, refers to a C₁-C₄alkyl group attached to the parent molecular moiety through an oxygen atom.

The term “C₁-C₆alkoxy”, as used herein, refers to a C₁-C₆alkyl group attached to the parent molecular moiety through an oxygen atom.

The term “C₁-C₁₃alkoxy”, as used herein, refers to a C₁-C₁₃alkyl group attached to the parent molecular moiety through an oxygen atom.

The term “C₁-C₃alkoxyC₁-C₃alkyl”, as used herein, refers to a C₁-C₃alkoxy group attached to the parent molecular moiety through a C₁-C₃alkyl group.

The term “C₁-C₁₃alkoxycarbonyl”, as used herein, refers to a C₁-C₁₃alkoxy group attached to the parent molecular moiety through a carbonyl group.

The term “C₁-C₃alkoxycarbonylC₁-C₃alkyl”, as used herein, refers to a C₁-C₃alkoxycarbonyl group attached to the parent molecular moiety through a C₁-C₃alkyl group.

The term “C₁-C₆alkoxycarbonylC₁-C₃alkyl”, as used herein, refers to a C₁-C₆alkoxycarbonyl group attached to the parent molecular moiety through a C₁-C₃alkyl group.

The term “C₁-C₃alkyl”, as used herein, refers to a group derived from a straight or branched chain saturated hydrocarbon containing from one to three carbon atoms.

The term “C₁-C₄alkyl”, as used herein, refers to a group derived from a straight or branched chain saturated hydrocarbon containing from one to four carbon atoms.

The term “C₁-C₆alkyl”, as used herein, refers to a group derived from a straight or branched chain saturated hydrocarbon containing from one to six carbon atoms.

The term “C₁-C₇alkyl”, as used herein, refers to a group derived from a straight or branched chain saturated hydrocarbon containing from one to seven carbon atoms.

The term “C₁-C₁₃alkyl”, as used herein, refers to a group derived from a straight or branched chain saturated hydrocarbon containing from one to thirteen carbon atoms.

The term “C₄-C₁₃alkyl”, as used herein, refers to a group derived from a straight or branched chain saturated hydrocarbon containing from four to thirteen carbon atoms.

The term “C₁-C₃alkylcarbonyl”, as used herein, refers to a C₁-C₃alkyl group attached to the parent molecular moiety through a carbonyl group.

The term “C₁-C₁₃alkylcarbonyl”, as used herein, refers to a C₁-C₁₃alkyl group attached to the parent molecular moiety through a carbonyl group.

The term “C₄-C₁₃alkylcarbonyl”, as used herein, refers to a C₄-C₁₃alkyl group attached to the parent molecular moiety through a carbonyl group.

The term “C₁-C₃alkylsulfanyl”, as used herein, refers to a C₁-C₃alkyl group attached to the parent molecular moiety through a sulfur atom.

The term “C₁-C₁₃alkylsulfanyl”, as used herein, refers to a C₁-C₁₃alkyl group attached to the parent molecular moiety through a sulfur atom.

The term “C₁-C₃alkylsulfanylC₁-C₃alkyl”, as used herein, refers to a C₁-C₃alkylsulfanyl group attached to the parent molecular moiety through a C₁-C₃alkyl group.

The term “C₁-C₁₃alkylsulfanylcarbonyl”, as used herein, refers to a C₁-C₁₃alkylsulfanyl group attached to the parent molecular moiety through a carbonyl group.

The term “C₁-C₃alkylsulfonyl”, as used herein, refers to a C₁-C₃alkyl group attached to the parent molecular moiety through a sulfonyl group.

The term “C₁-C₃alkylsulfonylamino”, as used herein, refers to a C₁-C₃alkylsulfonyl group attached to the parent molecular moiety through an amino group.

The term “amido”, as used herein, refers to —C(O)NH₂.

The term “amidoC₁-C₃alkyl”, as used herein, refers to an amido group attached to the parent molecular moiety through a C₁-C₃alkyl group.

The term “amino”, as used herein, refers to —NH₂.

The term “aminoC₁-C₃alkyl”, as used herein, refers to an amino group attached to the parent molecular moiety through a C₁-C₃alkyl group.

The term “aminosulfonyl”, as used herein, refers to an amino group attached to the parent molecular moiety through a sulfonyl group.

The term “azaindolylC₁-C₃alkyl”, as used herein, refers to an azaindolyl group attached to the parent molecular through a C₁-C₃alkyl group. The azaindolyl group can be attached to the alkyl moiety through any substitutable atom in the group.

The term “benzothiazolylC₁-C₃alkyl”, as used herein, refers to an benzothiazolyl group attached to the parent molecular through a C₁-C₃alkyl group. The benzothiazolyl group can be attached to the alkyl moiety through any substitutable atom in the group.

The term “benzothienylC₁-C₃alkyl”, as used herein, refers to a benzothienyl group attached to the parent molecular through a C₁-C₃alkyl group. The benzothienyl group can be attached to the alkyl moiety through any substitutable atom in the group.

The term “benzyl”, as used herein, refers to a phenyl group attached to the parent molecular moiety through a CH₂ group.

The term “benzyloxy”, as used herein, refers to a benzyl group attached to the parent molecular moiety through an oxygen atom.

The term “benzyloxyC₁-C₃alkyl”, as used herein, refers to a benzyloxy group attached to the parent molecular moiety through a C₁-C₃alkyl group.

The term “biphenylC₁-C₃alkyl”, as used herein, refers to a biphenyl group attached to the parent molecular moiety through a C₁-C₃alkyl group. The biphenyl group can be attached to the alkyl moiety through any substitutable atom in the group.

The term “carbonyl”, as used herein, refers to —C(O)—.

The term “carboxy”, as used herein, refers to —CO₂H.

The term “carboxyC₁-C₃alkyl”, as used herein, refers to a carboxy group attached to the parent molecular moiety through a C₁-C₃alkyl group.

The term “cyano”, as used herein, refers to —CN.

The term “C₃-C₁₄cycloalkyl”, as used herein, refers to a saturated monocyclic or bicyclic hydrocarbon ring system having three to fourteen carbon atoms and zero heteroatoms. The bicyclic rings can be fused, spirocyclic, or bridged. Representative examples of cycloalkyl groups include, but are not limited to, cyclopropyl, cyclopentyl, octahydropentalene, and bicyclo[3.1.1]heptyl.

The term “C₃-C₁₄cycloalkylC₁-C₃alkyl”, as used herein, refers to a C₃-C₁₄cycloalkyl group attached to the parent molecular moiety through a C₁-C₃alkyl group.

The term “C₃-C₁₄cycloalkylcarbonyl”, as used herein, refers to a C₃-C₁₄ cycloalkyl group attached to the parent molecular moiety through a carbonyl group.

The term “diphenylmethyl”, as used herein, refers to (Ph)₂CH—, wherein each Ph is a phenyl ring.

The term “furanylC₁-C₃alkyl”, as used herein, refers to a furanyl group attached to the parent molecular moiety through a C₁-C₃alkyl group. The furanyl group can be attached to the alkyl moiety through any substitutable atom in the group.

The term “furanylcarbonyl”, as used herein, refers to a furanyl group attached to the parent molecular moiety through a carbonyl group.

The terms “halo” and “halogen”, as used herein, refer to F, Cl, Br, or I.

The term “haloC₁-C₁₃alkoxy”, as used herein, refers to a haloC₁-C₁₃alkyl group attached to the parent molecular moiety through an oxygen atom

The term “haloC₁-C₁₃alkoxycarbonyl”, as used herein, refers to a haloC₁-C₁₃alkoxy group attached to the parent molecular moiety through a carbonyl group.

The term “haloC₁-C₃alkyl”, as used herein, refers to a C₁-C₃alkyl group substituted with one, two, or three halogen atoms.

The term “haloC₁-C₁₃alkyl”, as used herein, refers to a C₁-C₁₃alkyl group substituted with one, two, three, four, five, six, seven, eight, or nine halogen atoms.

The term “haloC₁-C₁₃alkylcarbonyl”, as used herein, refers to a haloC₁-C₁₃alkyl attached to the parent molecular moiety through a carbonyl group.

The term “heterocyclyl”, as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a four-to six-membered aromatic or non-aromatic carbocyclic ring or another monocyclic heterocyclyl group. The heterocyclyl groups of the present disclosure are attached to the parent molecular moiety through a carbon atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “hydroxy”, as used herein, refers to —OH.

The term “imidazolylC₁-C₃alkyl”, as used herein, refers to an imidazolyl group attached to the parent molecular moiety through a C₁-C₃alkyl group. The imidazolyl group can be attached to the alkyl moiety through any substitutable atom in the group.

The term “indolylC₁-C₃alkyl”, as used herein, refers to an indolyl group attached to the parent molecular moiety through a C₁-C₃alkyl group. The indolyl group can be attached to the alkyl moiety through any substitutable atom in the group.

The term “isoquinolinyloxy”, as used herein, refers to an isoquinoline group attached to the parent molecular moiety through an oxygen atom. The isoquinoline group can be attached to the oxygen atom through any substitutable carbon atom in the group.

The term “naphthylC₁-C₃alkyl”, as used herein, refers to a naphthyl group attached to the parent molecular moiety through a C₁-C₃alkyl group. The naphthyl group can be attached to the alkyl moiety through any substitutable atom in the group.

The term “nitro”, as used herein, refers to —NO₂.

The term “NR^{x}R^{y}”, as used herein, refers to two groups, R^{x} and R^{y}, which are attached to the parent molecular moiety through a nitrogen atom. R^{x} and R^{y} are independently selected from hydrogen, C₂-C₄alkenyloxycarbonyl, C₁-C₃alkylcarbonyl, C₃-C₁₄cycloalkylcarbonyl, furanylcarbonyl, and phenylcarbonyl.

The term “NR^{x}R^{y}(C₁-C₇)alkyl”, as used herein, refers to an NR^{x}R^{y} group attached to the parent molecular moiety through a C₁-C₇alkyl group.

The term “NR^{t}R^{u}”, as used herein, refers to two groups, R^{t} and R^{u}, which are attached to the parent molecular moiety through a nitrogen atom. R^{t} and R^{u} are independently selected from hydrogen, C₁-C₃alkyl, and triphenylmethyl.

The term “NR^{t}R^{u}carbonyl”, as used herein, refers to an NR^{t}R^{u} group attached to the parent molecular moiety through a carbonyl group.

The term “NR^{t}R^{u}carbonylC₁-C₃alkyl”, as used herein, refers to an NR^{t}R^{u}carbonyl group attached to the parent molecular moiety through a C₁-C₃alkyl group.

The tem “phenoxy”, as used herein, refers to a phenyl group attached to the parent molecular moiety through an oxygen atom.

The term “phenoxyC₁-C₃alkyl”, as used herein, refers to a phenoxy group attached to the parent molecular moiety through a C₁-C₃alkyl group.

The term “phenylC₁-C₃alkyl”, as used herein, refers to a phenyl group attached to the parent molecular moiety through a C₁-C₃alkyl group.

The term “phenylcarbonyl”, as used herein, refers to a phenyl group attached to the parent molecular moiety through a carbonyl group.

The term “pyridinylC₁-C₃alkyl”, as used herein, refers to a pyridinyl group attached to the parent molecular moiety through a C₁-C₃alkyl group. The pyridinyl group can be attached to the alkyl moiety through any substitutable atom in the group.

The term “quinolinyloxy”, as used herein, refers to a quinoline group attached to the parent molecular moiety through an oxygen atom. The quinoline group can be attached to the oxygen atom through any substitutable carbon atom in the group.

The term “sulfanyl”, as used herein, refers to —S—.

The term “sulfonyl”, as used herein, refers to —SO₂—.

The term “thiazolylC₁-C₃alkyl”, as used herein, refers to a thiazolyl group attached to the parent molecular moiety through a C₁-C₃alkyl group. The thiazolyl group can be attached to the alkyl moiety through any substitutable atom in the group.

The term “thienylC₁-C₃alkyl”, as used herein, refers to a thienyl group attached to the parent molecular moiety through a C₁-C₃alkyl group. The thienyl group can be attached to the alkyl moiety through any substitutable atom in the group.

The term “triphenylmethyl”, as used herein, refers to —C(Ph)₃, wherein each Ph is a phenyl group.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

### II. Pharmaceutical Compositions

In some aspects, the present disclosure provides compositions comprising cyclic peptides. The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable carrier” it is meant the carrier, diluent or excipient is compatible with the other ingredients of the formulation and not deleterious to the recipient thereof.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, minitablets, capsules, pills, troches, lozenges, pastilles, sachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be formulated as a tablet. In some aspects, the tablets can be encapsulated into capsules for administration.

The tablets of the disclosure can be in the form of compressed tablets, tablet triturates, chewable lozenges, rapidly dissolving tablets, multiple compressed tablets, or enteric-coated tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of enteric coated tablet.

In some aspects, the compositions of the disclosure can be prepared by dry granulation of the compound of the disclosure with one or more pharmaceutically acceptable carriers, vehicles, and/or excipients. In some aspects, the compositions of the disclosure can be prepared by wet granulation.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, tsoft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. The capsules can also be coated as known by those of skill in the art in order to modify or sustain dissolution of the active ingredient.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

The compositions described herein are typically part of an admixture with suitable pharmaceutical diluents, excipients, and/or carriers (collectively referred to herein as pharmaceutical carriers) suitably selected with respect to the intended form of administration, and consistent with conventional pharmaceutical practices. For instance, for oral administration in the form of a tablet or capsule, the active drug component can be combined with an oral, non-toxic, pharmaceutically acceptable, inert carriers. Moreover, when desired or necessary, suitable binders, lubricants, surfactants, disintegrating agents, glidants, flavoring agents, and coloring agents can also be incorporated into the mixture. Examples of these types of additives include, but are not limited to, lactose, sucrose, dextrose, dextrates, glucose, maltodextrin, mannitol, xylitol, sorbitol, cyclodextrins, calcium phosphate, calcium sulfate, natural starch, a pregelatinized starch, a sodium starch, methyl crystalline cellulose, methylcellulose, microcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, a gum, silicas, silicon dioxide, talc, pre-gelatinized starch, corn starch, magnesium aluminum silicates, sodium lauryl sulfate, sorbitan monooleate, polyoxyethylene sorbitan monooleate, polysorbates, polaxomers, bile salts, glyceryl monostearate, copolymers of ethylene oxide, propylene oxide, stearic acid, calcium hydroxide, talc, corn starch, sodium stearyl fumerate, stearic acid, sodium oleate, sodium stearate, sodium benzoate, sodium acetate, sodium chloride, magnesium stearate, zinc stearate, waxes, talc, and the like, as well as combinations thereof. In some aspects, the compositions described herein comprise, microcrystalline cellulose, mannitol, croscarmellose sodium, silicon dioxide, magnesium stearate, or a combination thereof.

In some aspects, the composition comprises from about 1% (w/w) to about 6% (w/w) of one or more fillers. In some aspects, the term “filler” refers to an inactive substance used to make an active ingredient bigger or easier to handle. Examples of fillers include, but are not limited to, lactose, sucrose, microcrystalline cellulose, calcium carbonate, and calcium phosphate. In some aspects, the composition comprises from about 1% (w/w) wt % to 5% (w/w) of one or more fillers. In some aspects, the composition comprises about 1%, about 2%, about 3%, about 4%, about 5%, or about 6% (w/w) of one or more fillers.

In some aspects, the one or more fillers comprises microcrystalline cellulose. In some aspects, the composition comprises from about 1% (w/w) to about 6% (w/w) microcrystalline cellulose. In some aspects, the composition comprises from about 1% (w/w) wt % to 5% (w/w) microcrystalline cellulose. In some aspects, the composition comprises about 1%, about 2%, about 3%, about 4%, about 5%, or about 6% (w/w) microcrystalline cellulose.

In some aspects, the composition comprises about 1% (w/w) to about 6% (w/w) of one or more disintegrants. In some aspects, the term “disintegrant” refers to an agent added to a formulation, in particular a tablet, to promote the break-up of the tablet into smaller fragment in an aqueous environment. Examples of disintegrants include, but are not limited to, croscarmellose sodium, crospovidone, corn starch, pre-gelatinized starch, and sodium starch glycolate. In some aspects, the composition comprises about 1% (w/w) wt % to 5% (w/w) of one or more disintegrants. In some aspects, the composition comprises about 1%, about 2%, about 3%, about 4%, about 5%, or about 6% (w/w) of one or more disintegrants.

In some aspects, the one or more disintegrants comprises croscarmellose sodium. In some aspects, the composition comprises from about 1% (w/w) to about 6% (w/w) croscarmellose sodium. In some aspects, the composition comprises from about 1% (w/w) wt % to 5% (w/w) croscarmellose sodium. In some aspects, the composition comprises about 1%, about 2%, about 3%, about 4%, about 5%, or about 6% (w/w) croscarmellose sodium.

In some aspects, the composition comprises about 1% (w/w) to about 6% (w/w) silicon dioxide. In some aspects, the composition comprises about 1% (w/w) to about 5% (w/w) silicon dioxide. In some aspects, the composition comprises about 1%, about 2%, about 3%, about 4%, about 5%, or about 6% (w/w) silicon dioxide.

In some aspects, the composition comprises about 1% (w/w) to about 6% (w/w) of one or more glidants. In some aspects, the term “glidant” refers to an agent used to increase powder flow. Examples of glidants include, but are not limited to, talc, magnesium stearate and other stearate salts, stearyl fumarate, stearic acid, and silica. In some aspects, the composition comprises about 1% (w/w) to about 5% (w/w) of one or more glidants. In some aspects, the composition comprises about 1%, about 2%, about 4%, about 4%, about 5%, or about 6% (w/w) of one or more glidants.

In some aspects, the one or more glidants comprises magnesium stearate. In some aspects, the composition comprises about 1% (w/w) to about 6% (w/w) magnesium stearate. In some aspects, the composition comprises about 1% (w/w) to about 5% (w/w) magnesium stearate. In some aspects, the composition comprises about 1%, about 2%, about 4%, about 4%, about 5%, or about 6% (w/w) magnesium stearate.

In still other aspects, using standard coating procedures, such as those described in Remington's Pharmaceutical Sciences, 20th Edition (2000), a film coating can be provided around the formulation of the compounds described herein.

Dosage forms (pharmaceutical compositions) suitable for administration can contain from about 1 milligram to about 300 milligrams of active ingredient per dosage unit. In these pharmaceutical compositions the active ingredient will ordinarily be present in an amount of about 0.5-95% by weight based on the total weight of the composition. In some aspects, dosage forms suitable for administration can contain from about 10 to about 240 milligrams of active ingredient per dosage unit. In some aspects, dosage forms suitable for administration can contain about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, or about 240 mg of active ingredient per dosage unit.

In certain aspects, the present disclosure provides a pharmaceutical composition comprising a biologically active compound, a salcaprozate salt, and nicotinamide. In some aspects, the present disclosure provides a pharmaceutical composition comprising a barrier that prevents drug release in an acidic environment, such as in gastric fluid. The biologically active compound can be any compound that exerts a direct physiological effect on a living thing. In certain aspects, the biologically active compound is one whose bioavailability improves when administered as a pharmaceutical composition described herein. In certain aspects, the biologically active compound is one whose oral bioavailability improves when administered as a pharmaceutical composition described herein. In certain aspects, the biologically active compound comprises a cyclic peptide. In certain aspects, the cyclic peptide can comprise from 5 to 30 amino acids. In some aspects, the cyclic peptide can comprise 5 to 20 amino acids. In some aspects, the cyclic peptide can comprise 12 to 16 amino acids. In certain aspects, the cyclic peptide backbone can comprise 2 to 20 amino acids. In certain aspects, the cyclic peptide backbone can comprise 4 to 18 amino acids. In certain aspects, the cyclic peptide backbone can comprise 6 to 16 amino acids. In certain aspects, the cyclic peptide backbone can comprise 8 to 14 amino acids. In certain aspects, the cyclic peptide backbone can comprise 10 to 14 amino acids. In certain aspects, the cyclic peptide backbone can comprise 2, 3, 4, 5, 6, 7, 8, 9, 10, 11, 12, 13, 14, 15, 16, 17, 18, 19, or 20 amino acids.

In certain aspects, the cyclic peptide can comprise a compound of formula (I):

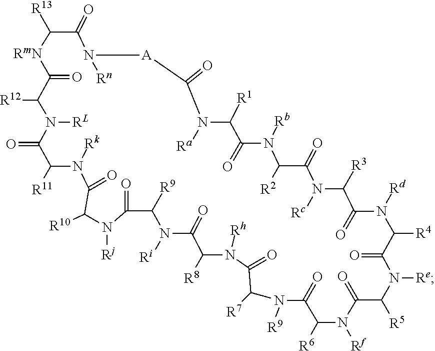

(I);
or a pharmaceutically acceptable salt thereof, wherein:
A is selected from:

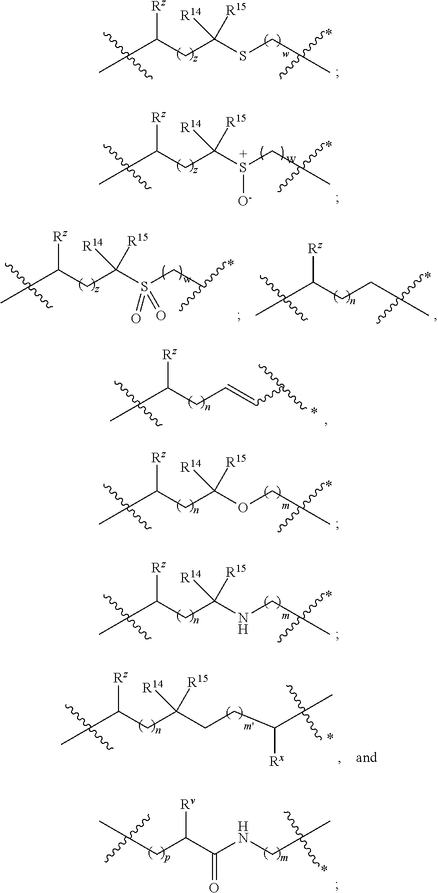

wherein

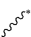

denotes the point of attachment to the carbonyl group and denotes

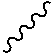

the point of attachment to the nitrogen atom;
n is 0, 1, or 2;
m is 1 or 2;
m′ is 0 or 1;
z is 0, 1 or 2;
w is 1 or 2;
p is 0, 1, or 2;
R¹⁴ and R¹⁵ are independently selected from hydrogen and methyl;
R^{x} is selected from hydrogen, amino, hydroxy, and methyl;
R^{v} is hydrogen, methyl, or a natural amino acid side chain;
R^{z} is selected from hydrogen and —C(O)NHR¹⁶;
R¹⁶ is selected from hydrogen, —CHR¹⁷C(O)NH₂, —CHR¹⁷C(O)NHCHR¹⁷C(O)NH₂, —CH₂C(O)NHCH(R¹⁷)C(O)NHCH(R^{17a})C(O)NH₂; —CH₂C(O)NHCH(R¹⁷)CO₂H; and —(C(R^{17a})₂)₂—X′—R³⁰;
each R¹⁷ is independently selected from hydrogen, —CH₃, —CH₂OH, and —(CH₂)_{w}-triazolyl-X—R³⁵;
R³⁵ is selected from —CO₂H and CH₃;
each R^{17a} is independently selected from hydrogen and —CH₂CO₂H;
X′ is a chain of between 8 and 46 atoms wherein the atoms are selected from carbon and oxygen and wherein the chain may contain one, two, or three C(O)NH groups embedded therein; and wherein the chain is optionally substituted with one or two groups independently selected from —CO₂H, —C(O)NH₂, —CH₂C(O)NH₂, and —CH₂CO₂H;
R³⁰ is selected from —CO₂H, —C(O)NR^{w}R^{x}, and —CH₃ wherein R^{w} and R^{w′} are independently selected from hydrogen and C₁-C₆alkyl, provided that when X′ is all carbon, R³⁰ is other than —CH₃;
X is selected from
—(CH₂)₂CH(CO₂H)NHC(O)(CH₂)_{f};
—(CH₂CH₂O)_{g}; and
—(CH₂CH₂O)_{g}CH₂CH₂NHC(O)CH₂CH₂CH(CO₂H)NHC(O)(CH₂)_{f};
f is 14, 15, or 16;
g is 3, 4, 5, 6, 7, 8, 9, 10, or 11;
R¹³ is selected from a natural amino acid, an unnatural amino acid, —(C(R^{17a})₂)₂—X′—R³⁰, and —(CH₂)_{w}-triazolyl-X—R³⁵;
R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{a}, R^{c}, R^{f}, R^{h}, Rⁱ, R^{j}, R^{m}, and Rⁿ are each independently selected from hydrogen and methyl;
R^{b} is hydrogen or methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R^{e} is hydrogen or methyl, or R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group;
R^{k} is hydrogen or methyl, or, R^{k} and R¹¹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, and hydroxy; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

In certain aspects, the cyclic peptide can comprise a compound of formula (I) wherein R¹⁶ is —CH₂C(O)NHCH(R¹⁷)CO₂H or —(C(R^{17a})₂)₂—X′—R³⁰; and R¹⁷ is —(CH₂)_{w}-triazolyl-X—R³⁵.

In certain aspects, the cyclic peptide can comprise a compound of formula (II):

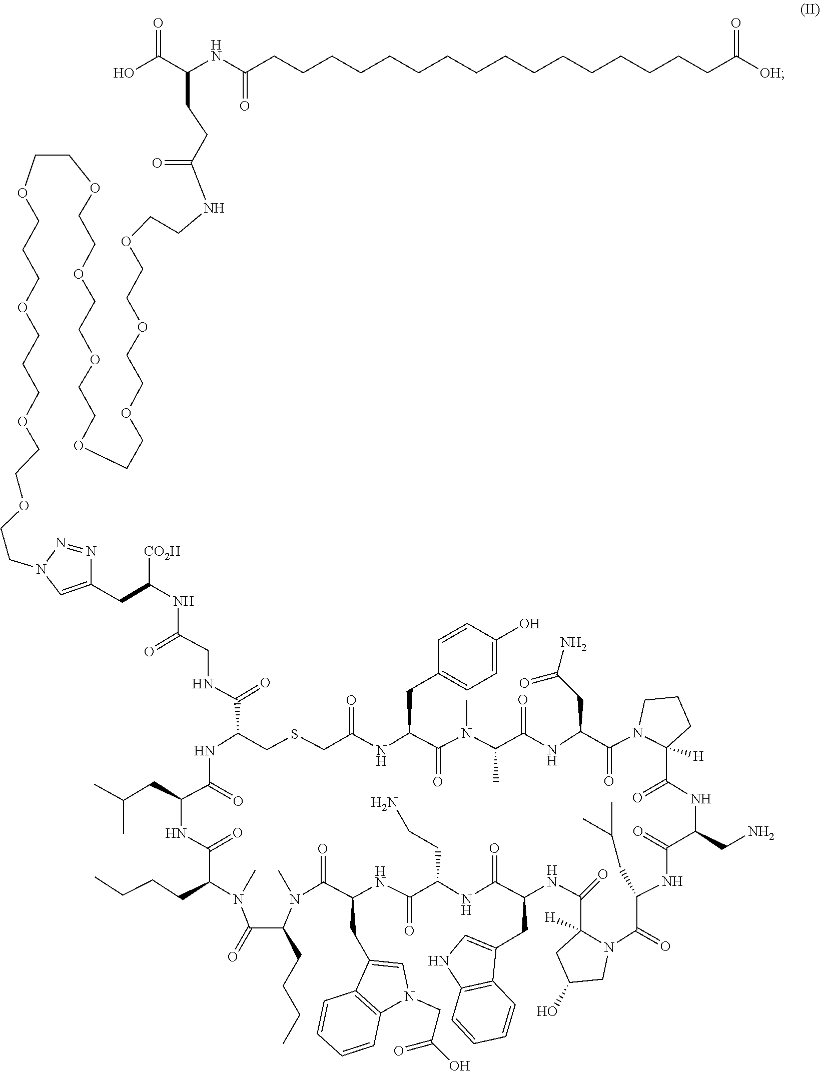 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately by reacting a free base function of the compound with a suitable acid or by reacting an acidic group of the compound with a suitable base. Acid addition salts include those derived from nontoxic inorganic acids, such as acetic, succinic, fumaric, hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In certain aspects, the biologically active compound is present in an amount of about 0.1% (w/w) to about 50% (w/w). In certain aspects, the biologically active compound is present in an amount of about 0.25% (w/w) to about 50% (w/w). In certain aspects, the biologically active compound is present in an amount of about 0.5% (w/w) to about 50% (w/w). In certain aspects, the biologically active compound is present in an amount of about 0.75% (w/w) to about 50% (w/w). In some aspects, the biologically active compound is present in an amount of about 1% (w/w) to about 50% (w/w). In some aspects, the biologically active compound is present in an amount of about 1% (w/w) to about 45% (w/w). In some aspects, the biologically active is present in an amount of about 2% (w/w) to about 40% (w/w). In some aspects, the biologically active is present in an amount of about 3% (w/w) to about 30% (w/w). In some aspects, the biologically active is present in an amount of about 4% (w/w) to about 20% (w/w). In some aspects, the biologically active is present in an amount of about 5% (w/w) to about 10% (w/w). In some aspects, the biologically active compound is present in an amount of about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 21% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), or about 45% (w/w).

The pharmaceutical compositions described herein can comprise a salcaprozate salt. In certain aspects, the salt can be sodium, potassium, magnesium, calcium, or tromethamine/tris(hydroxymethyl)aminomethane. In some aspects, the salt can be sodium.

In certain aspects, the salcaprozate salt is present in an amount of about 30% (w/w) to about 95% (w/w). In some aspects, the salcaprozate salt is present in an amount of about 50% (w/w) to about 90% (w/w). In some aspects, the salcaprozate salt is present in an amount of about 60% (w/w) to about 85% (w/w). In some aspects, the salcaprozate salt is present in an amount of about 60% (w/w) to about 80% (w/w). In some aspects, the salcaprozate salt is present in an amount of about 10% (w/w), about 12% (w/w), about 14% (w/w), about 16% (w/w), about 18% (w/w), about 20% (w/w), about 22% (w/w), about 24% (w/w), about 26% (w/w), about 28% (w/w), about 30% (w/w), about 32% (w/w), about 34% (w/w), about 36% (w/w), about 38% (w/w), about 40% (w/w), about 42% (w/w), about 44% (w/w), about 46% (w/w), about 48% (w/w), about 50% (w/w), about 52% (w/w), about 54% (w/w), about 56% (w/w), about 58% (w/w), about 60% (w/w), about 62% (w/w), about 64% (w/w), about 66% (w/w), about 68% (w/w), about 70% (w/w), about 72% (w/w), about 74% (w/w), about 76% (w/w), about 78% (w/w), about 80% (w/w), about 82% (w/w), about 84% (w/w), about 86% (w/w), about 88% (w/w), about 90% (w/w), about 92% (w/w), about 94% (w/w), or about 96% (w/w).

In certain aspects, the biologically active compound and the salcaprozate salt are present in a w/w ratio of about 0.01 to about 2. In certain aspects, the biologically active compound and the salcaprozate salt are present in a w/w ratio of about 0.02 to about 1.5. In some aspects, the biologically active compound and the salcaprozate salt are present in a w/w ratio of about 0.03 to about 1.4. In some aspects, the biologically active compound and the salcaprozate salt are present in a w/w ratio of about 0.01, about 0.02, about 0.03, about 0.04, about 0.05, about 0.06, about 0.07, about 0.08, about 0.09, about 0.10, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, about 1.5, about 1.6, about 1.7, about 1.8, about 1.9, or about 2.0.

In certain aspects, the pharmaceutical compositions described herein can comprise nicotinamide. In certain aspects, the nicotinamide can be present in an amount of from about 1% (w/w) to about 70% (w/w). In certain aspects, the nicotinamide can be present in an amount of from about 3% (w/w) to about 65% (w/w). In certain aspects, the nicotinamide can be present in an amount of from about 5% (w/w) to about 60% (w/w). In certain aspects, the nicotinamide can be present in an amount of from about 10% (w/w) to about 50% (w/w). In certain aspects, the nicotinamide can be present in an amount of from about 15% (w/w) to about 40% (w/w). In certain aspects, the nicotinamide can be present in an amount of from about 20% (w/w) to about 30% (w/w). In certain aspects, the nicotinamide can be present in an amount of about 1% (w/w), about 3% (w/w), about 5% (w/w), about 7% (w/w), about 9% (w/w), about 11% (w/w), about 13% (w/w), about 15% (w/w), about 17% (w/w), about 19% (w/w), about 21% (w/w), about 23% (w/w), about 25% (w/w), about 27% (w/w), about 29% (w/w), about 31% (w/w), about 35% (w/w), about 37% (w/w), 39% (w/w), about 41% (w/w), about 43% (w/w), about 45% (w/w), about 47% (w/w), about 49% (w/w), about 51% (w/w), about 53% (w/w), about 55% (w/w), about 57% (w/w), about 59% (w/w), about 61% (w/w), about 63% (w/w), about 65% (w/w), about 67% (w/w), about 69% (w/w), or about 70% (w/w).

In certain aspects, the pharmaceutical compositions can comprise one or more protease inhibitors. In some aspects, the one or more protease inhibitors comprises one or more trypsin inhibitors. In some aspects, the one or more trypsin inhibitors can be isolated from bovine pancreas, raw avian egg white, soybean, or lima bean. In some aspects, the one or more protease inhibitors can be selected from soybean trypsin inhibitor, aprotinin, lima bean trypsin inhibitor, ovomucoid trypsin inhibitor, and combinations thereof.

In some aspects, the one or more protease inhibitors can be present in an amount of about 0.1% (w/w) to about 50% (w/w). In some aspects, the one more protease inhibitors can be present in an amount of about 0.5% (w/w) to about 40% (w/w). In some aspects, the one more protease inhibitors can be present in an amount of about 0.75% (w/w) to about 30% (w/w). In some aspects, the one more protease inhibitors can be present in an amount of about 1% (w/w) to about 25% (w/w). In some aspects, the one more protease inhibitors can be present in an amount of about 5% (w/w) to about 20% (w/w). In some aspects, the one more protease inhibitors can be present in an amount of about 0.1% (w/w), about 0.2% (w/w), about 0.3% (w/w), about 0.4% (w/w), about 0.5% (w/w), about 0.6% (w/w), about 0.7% (w/w), 0.8% (w/w), about 0.9% (w/w), about 1.0% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), or about 50% (w/w).

In certain aspects, the present disclosure provides a pharmaceutical composition comprising a cyclic peptide at a concentration of about 0.1% (w/w) to about 50% (w/w); a salcaprozate salt at a concentration of about 30% (w/w) to about 95% (w/w); and nicotinamide at a concentration of about 5% (w/w) to about 60% (w/w). In some aspects, the salcaprozate salt can be salcaprozate sodium. In some aspects, the biologically active can be a cyclic peptide. In some aspects, the cyclic peptide can be a compound of formula (I), or a pharmaceutically acceptable salt thereof. In some aspects, the biologically active compound can be a compound of formula (I), or a pharmaceutically acceptable salt thereof, wherein R¹⁶ is —CH₂C(O)NHCH(R¹⁷)CO₂H or —(C(R^{17a})₂)₂—X′—R³⁰; and R¹⁷ is —(CH₂)_{w}-triazolyl-X—R³⁵. In some aspects, the biologically active compound can be a compound of formula (II), or a pharmaceutically acceptable salt thereof. In some aspects, the composition can comprise one or more protease inhibitors at a concentration of about 1% (w/w) and about 20% (w/w).

In certain aspects, the present disclosure provides a pharmaceutical composition comprising a cyclic peptide at a concentration of about 1% (w/w) to about 45% (w/w); a salcaprozate salt at a concentration of about 50% (w/w) to about 90% (w/w); and nicotinamide at a concentration of about 10% (w/w) to about 50% (w/w). In some aspects, the salcaprozate salt can be salcaprozate sodium. In some aspects, the biologically active can be a cyclic peptide. In some aspects, the cyclic peptide can be a compound of formula (I), or a pharmaceutically acceptable salt thereof. In some aspects, the biologically active compound can be a compound of formula (I), or a pharmaceutically acceptable salt thereof, wherein R¹⁶ is —CH₂C(O)NHCH(R¹⁷)CO₂H or —(C(R^{17a})₂)₂—X′—R³⁰; and R¹⁷ is —(CH₂)_{w}-triazolyl-X—R³⁵. In some aspects, the biologically active compound can be a compound of formula (II), or a pharmaceutically acceptable salt thereof. In some aspects, the composition can comprise one or more protease inhibitors at a concentration of about 1% (w/w) and about 20% (w/w).

In certain aspects, the present disclosure provides a pharmaceutical composition comprising a cyclic peptide at a concentration of about 2% (w/w) to about 40% (w/w); a salcaprozate salt at a concentration of about 60% (w/w) to about 85% (w/w); and nicotinamide at a concentration of about 15% (w/w) to about 40% (w/w). In some aspects, the salcaprozate salt can be salcaprozate sodium. In some aspects, the biologically active can be a cyclic peptide. In some aspects, the cyclic peptide can be a compound of formula (I), or a pharmaceutically acceptable salt thereof. In some aspects, the biologically active compound can be a compound of formula (I), or a pharmaceutically acceptable salt thereof, wherein R¹⁶ is —CH₂C(O)NHCH(R¹⁷)CO₂H or —(C(R^{17a})₂)₂—X′—R³⁰; and R¹⁷ is —(CH₂)_{w}-triazolyl-X—R³⁵. In some aspects, the biologically active compound can be a compound of formula (II), or a pharmaceutically acceptable salt thereof. In some aspects, the composition can comprise one or more protease inhibitors at a concentration of about 1% (w/w) and about 20% (w/w).

In certain aspects, the present disclosure provides a pharmaceutical composition comprising a cyclic peptide; a salcaprozate salt at a concentration of about 45% (w/w) to about 80% (w/w); and nicotinamide at a concentration of about 15% (w/w) to about 30% (w/w). In some aspects, the salcaprozate salt can be salcaprozate sodium. In some aspects, the cyclic peptide can be present at a concentration of about 1% (w/w) to about 45% (w/w). In some aspects, the biologically active can be a cyclic peptide. In some aspects, the cyclic peptide can be a compound of formula (I), or a pharmaceutically acceptable salt thereof. In some aspects, the biologically active compound can be a compound of formula (I), or a pharmaceutically acceptable salt thereof, wherein R¹⁶ is —CH₂C(O)NHCH(R¹⁷)CO₂H or —(C(R^{17a})₂)₂—X′—R³⁰; and R¹⁷ is —(CH₂)_{w}-triazolyl-X—R³⁵. In some aspects, the biologically active compound can be a compound of formula (II), or a pharmaceutically acceptable salt thereof. In some aspects, the composition can comprise one or more protease inhibitors at a concentration of about 1% (w/w) and about 20% (w/w).

In certain aspects, the present disclosure provides a composition comprising a compound of formula (II):

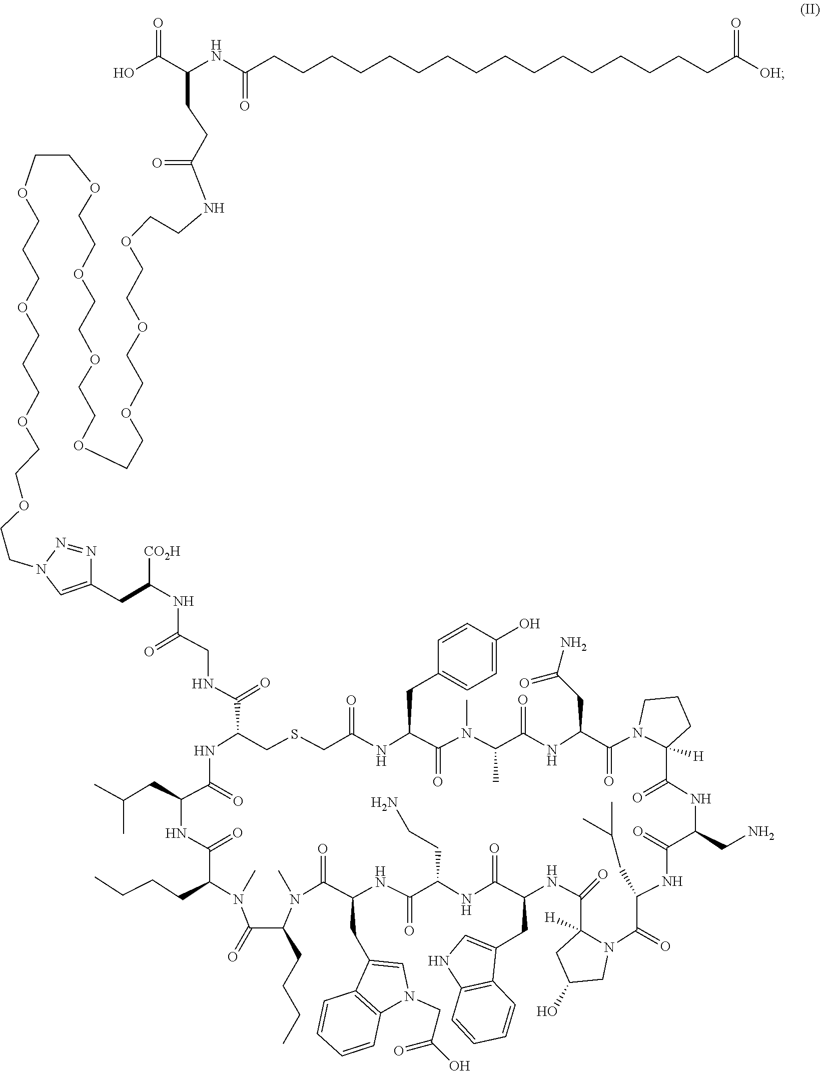 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof;
salcaprozate sodium, and nicotinamide, where the salcaprozate sodium and nicotinamide provide an oral bioavailability of >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

Also within the scope of the present disclosure are kits comprising the compositions of the disclosure and instructions for use. The kit can further contain additional reagents. Kits typically include a label indicating the intended use of the contents of the kit and instructions for use. The term label includes any writing, or recorded material supplied on or with the kit, or which otherwise accompanies the kit.

### III. Methods of Using

In some aspects, the present disclosure provides a method of improving oral bioavailability of a biologically active compound in a subject in need thereof comprising formulating the biologically active compound with a salcaprozate salt and nicotinamide. In some aspects, the salcaprozate salt is salcaprozate sodium (SNAC). In some aspects, the formulation further comprises one or more protease inhibitors.

In some aspects, the present disclosure provides pharmaceutical formulations that improvise the oral bioavailibity of biologically active compounds. In certain aspects, the oral bioavailability is improved by at least 5%. In some aspects, the oral bioavailability is improved by at least 10%. In some aspects, the oral bioavailability is improved by at least 15%. In some aspects, the oral bioavailability is improved by at least 20%. In some aspects, the oral bioavailability is improved by at least 25%. In some aspects, the oral bioavailability is improved by at least 30%. In some aspects, the oral bioavailability is improved by at least 35%. In some aspects, the oral bioavailability is improved by at least 40%. In some aspects, the oral bioavailability is improved by at least 45%. In some aspects, the oral bioavailability is improved by at least 50%. In some aspects, the oral bioavailability is improved by at least 55%. In some aspects, the oral bioavailability is improved by at least 60%. In some aspects, the oral bioavailability is improved by at least 65%. In some aspects, the oral bioavailability is improved by at least 70%. In some aspects, the oral bioavailability is improved by at least 75%. In some aspects, the oral bioavailability is improved by at least 80%. In some aspects, the oral bioavailability is improved by at least 90%. In some aspects, the oral bioavailability is improved by 100%. In some aspects, the oral bioavailability is improved by 125%. In some aspects, the oral bioavailability is improved by 150%. In some aspects, the oral bioavailability is improved by 175%. In some aspects, the oral bioavailability is improved by 200%. In some aspects, the oral bioavailability is improved by 225%. In some aspects, the oral bioavailability is improved by 250%. In some aspects, the oral bioavailability is improved by 275%. In some aspects, the oral bioavailability is improved by 300%. In some aspects, the oral bioavailability is improved by 325%. In some aspects, the oral bioavailability is improved by 350%. In some aspects, the oral bioavailability is improved by 375%. In some aspects, the oral bioavailability is improved by 400%. In some aspects, the oral bioavailability is improved by 425%. In some aspects, the oral bioavailability is improved by 450%. In some aspects, the oral bioavailability is improved by 475%. In some aspects, the oral bioavailability is improved by 500%. In some aspects, the oral bioavailability is improved by 525%. In some aspects, the oral bioavailability is improved by 550%. In some aspects, the oral bioavailability is improved by 575%. In some aspects, the oral bioavailability is improved by 600%. In some aspects, the oral bioavailability is improved by 625%. In some aspects, the oral bioavailability is improved by 650%. In some aspects, the oral bioavailability is improved by 675%. In some aspects, the oral bioavailability is improved by 700%. In some aspects, the oral bioavailability is improved by 725%. In some aspects, the oral bioavailability is improved by 750%. In some aspects, the oral bioavailability is improved by 775%. In some aspects, the oral bioavailability is improved by 800%.

In some aspects, the present disclosure provides a method of improving the oral bioavailability of a compound of formula (II):

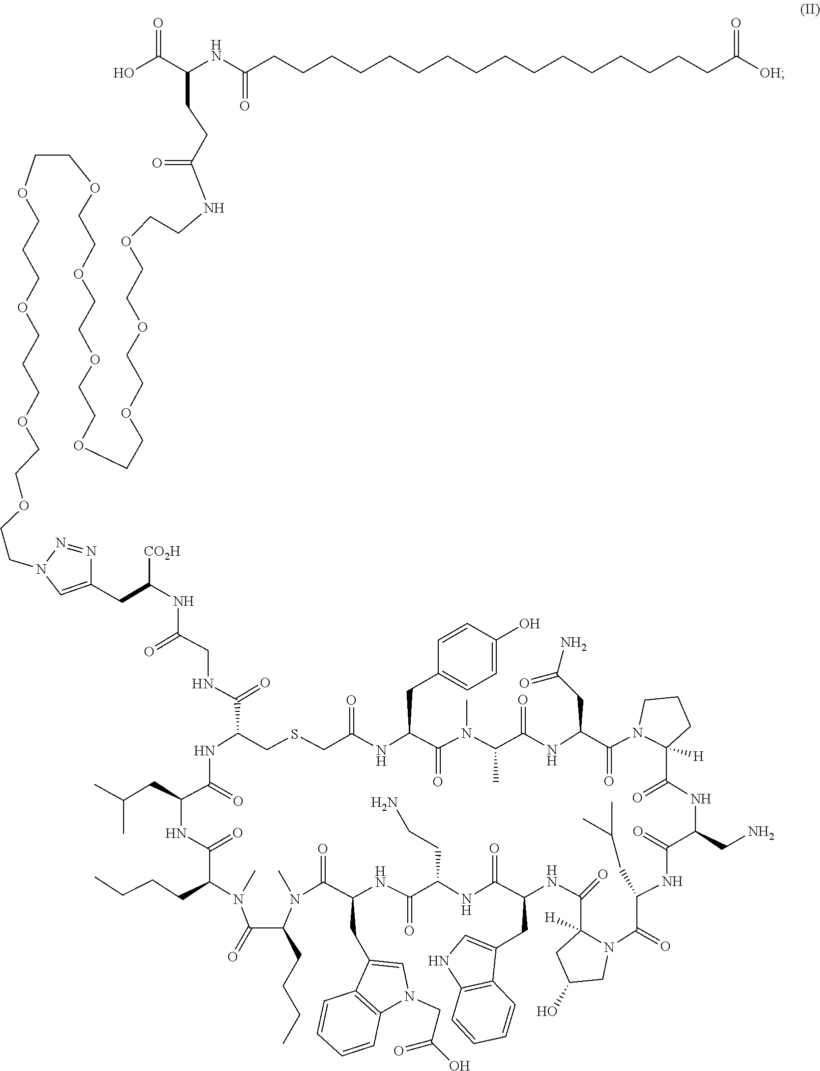 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]

or a pharmaceutically acceptable salt thereof, to >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%, the method comprising formulating the biologically active compound with a salcaprozate salt and nicotinamide.

Administration of a biologically active compound described herein includes, without limitation, administration of a therapeutically-effective amount of the compound. The term “therapeutically effective amount” as used herein refers, without limitation, to an amount of a biologically active compound to treat a condition treatable by administration of a composition comprising the compound. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration. Thus, it is not useful to specify an exact effective amount in advance.

In another aspect, the disclosure pertains to methods of inhibiting growth of tumor cells in a subject using the pharmaceutical compositions of the present disclosure. In some aspects, the biologically active compound is capable of binding to PD-L1, disrupting the interaction between PD-L1 and PD-1, competing with the binding of PD-L1 with anti-PD-1 monoclonal antibodies that are known to block the interaction with PD-1, enhancing CMV-specific T cell IFNγ secretion, and enhancing HIV-specific T cell IFNγ secretion. As a result, in some aspects, the biologically active compounds of the present disclosure are useful for modifying an immune response, treating diseases such as cancer, infectious disease, and/or septic shock, stimulating a protective autoimmune response or to stimulate antigen-specific immune responses.

Cancers whose growth can be inhibited using the pharmaceutical compositions of the present disclosure include, but are not limited to, cancers typically responsive to immunotherapy. Representative examples include melanoma (e.g., metastatic malignant melanoma), renal cell carcinoma, prostate cancer (including, but not limited to, castration-resistant prostate cancer), breast cancer, colon cancer and lung cancer (including, but not limited to, squamous non-small cell lung cancer, non-squamous non-small cell lung cancer). Examples of other cancers that can be treated using the methods of the present disclosure include bone cancer, hepatocellular carcinoma, pancreatic carcinoma, skin cancer, squamous cell carcinoma of the head and neck, cutaneous or intraocular malignant melanoma, uterine cancer, ovarian cancer, rectal cancer, cancer of the anal region, gastric cancer, gastrointestinal tract cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, carcinomas of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, hematological malignancies (such as chronic or acute leukemias including acute myeloid leukemia, chronic myeloid leukemia, acute lymphoblastic leukemia, chronic lymphocytic leukemia), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The pharmaceutical compositions described herein can also be useful for treatment of metastatic cancers.

The pharmaceutical compositions described herein can enhance, stimulate, and/or increase an immune response in a subject in need thereof. As used herein, the term “immune response” refers to the action of, for example, lymphocytes, antigen presenting cells, phagocytic cells, granulocytes, and soluble macromolecules produced by the above cells or the liver (including macrocyclic peptides, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In certain aspects, the immune response can be generated by the innanate immune system. In certain aspects, the immune response can be generated by the adaptive immune system. In certain embodiments, the immune response can be generated by the innate immune system and the adaptive immune system.

The pharmaceutical compositions described herein can be delivered by numerous methods including, but not limited to, orally, subcutaneously, intramuscularly, intraduodenally, or intravenously. As used herein, oral administration includes buccal, lingual, and sublingual administration

The composition can be formulated according to the route of administration based on acceptable pharmacy practice (Remington's Pharmaceutical Sciences, 23^{rd} Edition (2020)),

The dosage regimen for the compositions described herein will, of course, vary depending upon known factors, such as the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the disease state.

By way of general guidance, the daily oral dosage of the active ingredient, when used for the indicated effects, will range between about 0.001 to 500 mg/kg of body weight, preferably between about 0.01 to 100 mg/kg of body weight per day, and most preferably between about 0.1 to 20 mg/kg/day. Intravenously, the daily dosage of the active ingredient when used for the indicated effects will range between 0.001 ng to 100.0 ng per min/per Kg of body weight during a constant rate infusion. Such constant intravenous infusion can be preferably administered at a rate of 0.01 ng to 50 ng per min per Kg body weight and most preferably at 0.01 ng to 10.0 mg per min per Kg body weight. The compositions described herein may be administered in a single daily dose, or the total daily dosage may be administered in divided doses of two, three, or four times daily. In some aspects, the present disclosure provides a pharmaceutical composition comprising one or more dosage units.

### Biological Activity

Cyclic peptides can be prepared by methods known to those of skill in the art (see, for example, U.S. Pat. Nos. 9,308,236 and 9,856,292).

### Example 1—Regional Absorption Assessment of SNAC-Based Formulation

The pharmacokinetics of SNAC-based formulations of Compound (II) (prepared according to the procedure described in U.S. Pat. No. 9,856,292) were explored using two different formulations as shown in Table 1.

TABLE 1
Formulations Used in Absorption Assessment in Rats

| Formulation | Dosage Amount | Formulation Description |
|---|---|---|
| Formulation A | 0.4 mL formulation | 7.5 mg/mL Compound (II), |
| | (i.e., 3 mg | 375 mg/mL Salcaprozate Na |
| | Compound (II)) | (SNAC), 150 mg/mL |
| | | nicotinamide. |
| Formulation B | 0.4 mL formulation | 7.5 mg/mL Compound (II), |
| | (i.e., 3 mg | 100 mg/mL Salcaprozate Na |
| | Compound (II)) | (SNAC) |

The study was run as a two-treatment, two-segment, parallel study in anesthetized, fasted, male Sprague Dawley rats (rat weight: ˜300 g) with 4 rats per group.

For gastric absorptional testing, the subject formulation was dosed by oral gavage in rats with ligation at the pylorus to contain drug in the gastric compartment.

For duodenal absorptional testing, the rats were ligated at pylorus (proximal) and the subject formulation was injected directly into duodenum 5 cm past pylorus ligation (about 15 cm from the pylorus) and the distal part of the segment was ligated to contain drug in intestinal segment.

Samples of the dosed solution from gastric and duodenal segments were extracted at 60 min to measure remaining drug concentrations.

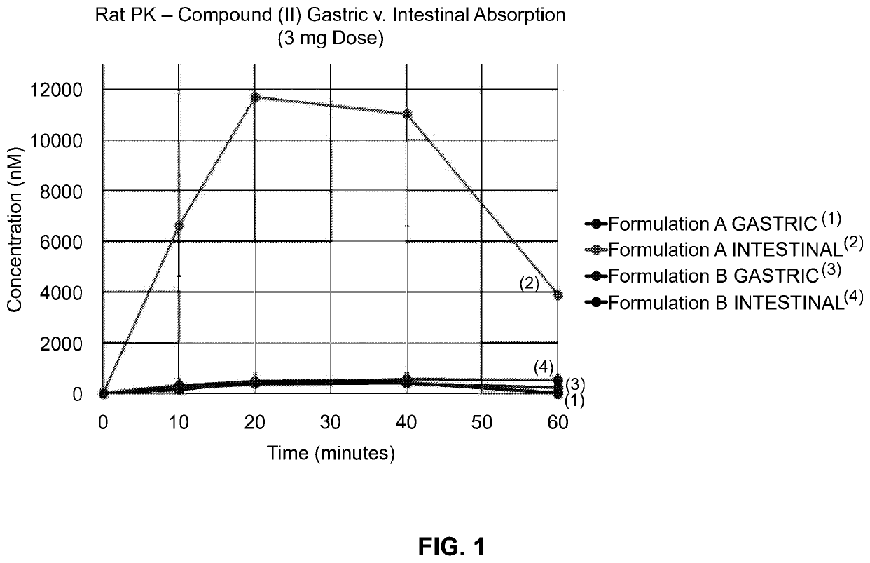

As shown in FIG. 1 and Table 1, the intestinal compartment offers good systemic absorption for Compound (II) compared to the gastric compartment. The formulation with the higher amount of SNAC and with nicotinamide shows >10× exposure in the intestine relative to lower SNAC concentration and no nicotinamide.

TABLE 2
Gastric and Intestinal Absorption of Formulations A and B of Compound (II) in Rats

| | Dose | Cmax (nM) | | Tmax (h) | AUC_{0-inf} (nM*h) | | |
|---|---|---|---|---|---|---|---|
| | (mg) | Mean | Std Dev | (Median) | Mean | Std Dev | CV (%) |
| Formulation A | 3 | 479.50 | 202.62 | 20.0 | 11333.75 | 3884.80 | 34.28 |
| GASTRIC | | | | | | | |
| Formulation A | 3 | 12450.00 | 2050.20 | 20.0 | 270025.00 | 185407.25 | 68.66 |
| INTESTINAL | | | | | | | |
| Formulation B | 3 | 425.25 | 418.43 | 60.0 | 14326.25 | 10808.43 | 75.44 |
| GASTRIC | | | | | | | |
| Formulation B | 3 | 577.00 | 243.84 | 40.0 | 24525.75 | 12295.95 | 50.13 |
| INTESTINAL | | | | | | | |

### Example 2—Preparation of Compound (II) Enteric Coated Tablets of 2.5 mg Strength for 20 mg Dose (8 Tablets Total)

The composition of uncoated core tablets is shown in Table 3. Compound (II), SNAC, and nicotinamide were combined in a mortar and blended using a pestle. The mixed blend was then added to a glass bottle along with the Magnesium stearate, and the mixture was blended for 5 minutes using a Turbula mixer (46 RPM, 5 minutes). The final mixture was compressed using a 7/32″ round standard concave tooling at 400 lb compression force at a target weight of 70 mg.

TABLE 3
Composition of Uncoated Core Tablets

| | | | |
|---|---|---|---|
| | | | Dose |
| Ingredient | % (w/w) | Batch Amt. (g) | Amt. (mg) |

Intra-granular Components

| Compound (II) | 2.15* | 0.321 | (0.362*) | 12 | (13.47*) |
|---|---|---|---|---|---|
| Salcaprozate sodium | 64.52 | 9.678 | | 360 | |
| Nicotinamide | 21.51 | 3.226 | (3.185**) | 120 | (118.47**) |

| | | | |
|---|---|---|---|
| Soybean trypsin | 8.06 | 1.209 | 45 |
| inhibitor | | | |
| Aprotinin | 2.69 | 0.404 | 15 |
| Magnesium stearate | 1.08 | 0.162 | 6 |
| Total | 100 | 15.000 | 558 |

*Amount of compound (II) after potency correction with an Assay “as is” value of 89%
**Amount of nicotinamide is adjusted to compensate for potency correction.

The composition of the seal coated tablet is shown in Table 4. An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 40 cfm; Spray rate: 5 g/min; Atomizing air pressure: 10.8 psi; Pattern air pressure: 10.8 psi; Inlet temperature: 55° C.; Outlet temperature: 40° C.; Pan speed: 20-25 rpm.

TABLE 4
Composition of Seal Coated Tablets

| Ingredient | % (w/w) | Batch Amt. (g) |
|---|---|---|
| Uncoated core tablets and placebo tablets* | 98.04 | 300 |
| Opadry 03K19229 | 1.96 | 6 |
| Total | 100.00 | 306 |

*Placebo tablets are added to achieve a suitable batch size for coating process.

The composition of the enteric coated tablet is shown in Table 5. An aqueous suspension of Acryl EZE II was prepared at 10% solid content. Compound (II) seal coated tablets and placebo tablets were added to a Vector 0.5-L coating pan. The tablets were pan coated to achieve 7% target weight gain. Four coated tablets were added in each #00 gray opaque hard gelatin capsule for the cyno PK study. Each cyno monkey was dosed with 2 capsules (8 coated tablets).

Air volume: 45 cfm; Spray rate: 5 g/min; Atomizing air pressure: 10.8 psi; Pattern air pressure: 10.8 psi; Inlet temperature: 55° C.; Outlet temperature: 40° C.; Pan speed: 20-25 rpm.

TABLE 5
Composition of Enteric Coating

| Ingredient | % (w/w) | Grams per batch |
|---|---|---|
| Compound (II) sealed tablets | 93.46 | 306 |
| (mixed with placebo tablets) | | |
| Acryl-EZE II 493Z180022 White | 6.54 | 21 |
| Total | 100.00 | 327 |

### Example 3—Determination of Oral Absorption of Prepared Formulations of Compound (II) in Cynologous Monkeys

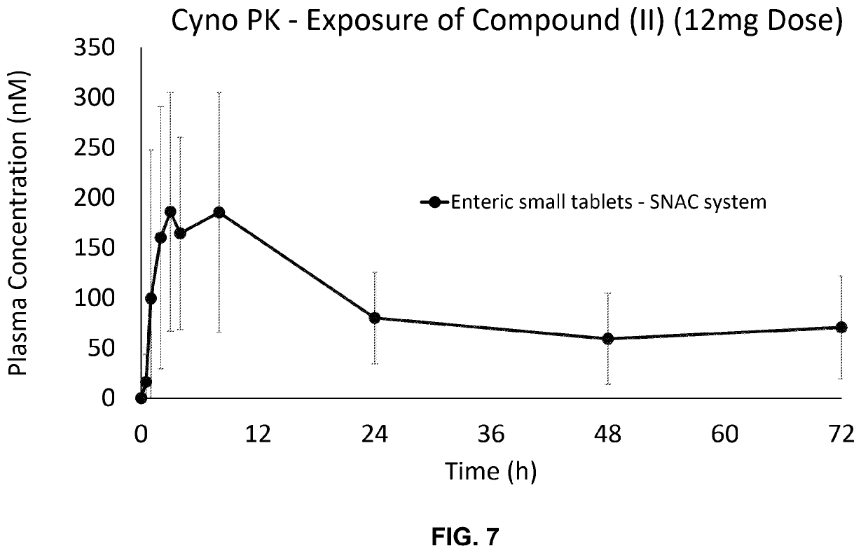

Fasted male cyno monkeys (n=3) were dosed by oral route followed by gavage flush with water to facilitate gastro-intestinal dissolution. Blood samples were taken at predetermined times over 72 hours post-dose to measure comparative pharmacokinetics between the formulations. Due to the long half-life these were conducted in a parallel group design in different sets of cyno monkeys. As shown in FIG. 7 and Tables 6A and 6B, the formulation provided good concentration of Compound (II) over an extended period of time.

TABLE 6A
Pharmacokinetic Profile of Formulated Tablet

| | | Cmax (nM) | | | AUC_{0-24 h} (nM · h) | | BA (%) | | |
|---|---|---|---|---|---|---|---|---|---|
| | Dose | | Std | Tmax (h) | | Std | | Std | CV |
| | (mg) | Mean | Dev | (Median) | Mean | Dev | Mean | Dev | (%) |
| 0.1 mpk IV | 0.5 | 798.33 | 78.80 | 0.3 | 47613.51 | 2131.26 | 100.00 | n/a | 4.48 |
| Example X | 12 | 190.73 | 118.46 | 4.0 | 3332.50 | 2157.88 | 1.34 | 0.87 | 64.75 |

TABLE 6B
Pharmacokinetic Profile of Formulated Tablet

| | | AUC_{inf} | | BA (%) using | | | AUC % | | | | | |
|---|---|---|---|---|---|---|---|---|---|---|---|---|
| | | (nM · h) | | AUC_{Inf} | | | Extrapolated | | C_{24 h} (nM) | | Cmin (nM) | |
| | Dose | | Std | | Std | CV | | Std | | Std | | Std |
| | (mg) | Mean | Dev | Mean | Dev | (%) | Mean | Dev | Mean | Dev | Mean | Dev |
| 0.1 mpk | 0.5 | 51965.95 | 2466.56 | 100.00 | n/a | 4.7 | 8.36 | 1.09 | 544.33 | 45.24 | 121.67 | 9.87 |
| IV | | | | | | | | | | | | |
| Example | 12 | 14385.95 | 7354.30 | 1.15 | 0.59 | 51.1 | 55.84 | 10.54 | 79.97 | 45.65 | 58.53 | 46.05 |
| X | | | | | | | | | | | | |

### Example 4—Preparation of Compound (II) Enteric Coated Tablets of 3.33 mg Strength for 20 mg Dose (6 Tablets Total)

The composition of uncoated core tablets is shown in Table 7. Compound (II), SNAC, and nicotinamide were combined and blended using a Turbula mixer (46 RPM, 10 minutes). Magnesium stearate was added and the mixture was blended for an additional 5 minutes. The final mixture was compressed using a 9/32″ round standard concave tooling at 700 lb compression force to form tablets at a target weight of 139.2 mg.

TABLE 7
Composition of Uncoated Core Tablets

| | | | |
|---|---|---|---|
| Ingredient | % (w/w) | Batch Amt. (g) | Dose Amt. (mg) |

Intra-granular Components

| Compound (II) | 2.4 | 0.24 | (0.27*) | 20 | (22.47*) |
|---|---|---|---|---|---|
| Salcaprozate sodium | 71.86 | 7.186 | | 600 | |
| Nicotinamide | 23.94 | 2.394 | (2.324**) | 220 | (197.53**) |

| | | | |
|---|---|---|---|
| Magnesium stearate | 1.8 | 0.18 | 15 |
| Total | 100 | 10 | 835 |

*Amount of compound (II) is adjusted based on Assay “as is” of 89%.
**Amount of nicotinamide is adjusted to compensate for potency correction.

The composition of the seal coated tablet is shown in Table 8 An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 40-50 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 8
Composition of Coated Tablet

| Ingredient | % (w/w) | Batch Amt. (g) |
|---|---|---|
| Uncoated core tablets and placebo tablets* | 98.04 | 300 |
| Opadry 03K19229 | 1.96 | 6 |
| Total | 100.00 | 306 |

*Placebo tablets are added to achieve a suitable batch size for coating process.

The composition of the enteric coated tablet is shown in Table 9. An aqueous suspension of Acryl EZE II was prepared at 10% solid content. Compound (II) seal coated tablets and placebo tablets were added to a Vector 0.5-L coating pan. The tablets were pan coated to achieve 7% target weight gain. Three enteric coated tablets were encapsulated into a size #00 hard gelatin capsule for the dog PK study. Each dog was dosed with 2 capsules (6 enteric coated tablets).

Air volume: 40-50 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 9
Composition of Enteric Coated Tablet

| Ingredient | % (w/w) | Batch Amt. (g) |
|---|---|---|
| Compound II seal coated | 93.58 | 306 |
| tablets and placebo tablets* | | |
| Acryl-EZE II 493Z180022 | 6.52 | 21 |
| Total | 100.00 | 327 |

*Placebo tablets are added to achieve a suitable batch size for coating process.

### Example 5—Preparation of Compound (II) Enteric Coated Tablets Containing Protease Inhibitor with 1.67 mg Strength for 20 mg Dose (12 Tablets Total)

The composition of uncoated core tablets is shown in Table 10. Compound (II), SNAC, nicotinamide, soybean trypsin inhibitor, and aprotinin were combined and blended. Magnesium stearate was added and the mixture was blended using a Turbula mixer (46 RPM, 5 minutes). The final mixture was compressed using a 7/32″ round standard concave tooling at 400 lb compression force to form tablets at a target weight of 78 mg.

TABLE 10
Composition of Uncoated Core Tablets

| | | | | Dose | |
|---|---|---|---|---|---|
| Ingredient | % (w/w) | Batch Amt. (g) | | Amt. (mg) | |
| Compound (II) | 2.15 | 0.215 | (0.242*) | 20 | (22.47*) |
| Salcaprozate sodium | 64.52 | 6.452 | | 600 | |
| Nicotinamide | 21.50 | 2.150 | (2.123**) | 200 | (197.53**) |

| | | | |
|---|---|---|---|
| Soybean trypsin | 8.06 | 0.806 | 75 |
| inhibitor | | | |
| Aprotinin | 2.69 | 0.269 | 25 |
| Magnesium stearate | 1.08 | 0.108 | 10 |
| Total | 100 | 10 | 930 |

*Amount of compound (II) is adjusted based on Assay “as is” of 89%.
**Amount of nicotinamide is adjusted to compensate for potency correction.

The composition of the seal coated tablet is shown in Table 11. An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 50 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 11
Composition of Seal Coated Tablet

| Ingredient | % (w/w) | Batch Amt. (g) |
|---|---|---|
| Uncoated core tablets and placebo tablets* | 98.04 | 300 |
| Opadry 03K19229 | 1.96 | 6 |
| Total | 100.00 | 306 |

*Placebo tablets are added to achieve a suitable batch size for coating process.

The composition of the enteric coated tablet is shown in Table 12. An aqueous suspension of Acryl EZE II was prepared at 10% solid content. Compound (II) seal coated tablets (8 g) and 292 g of placebo tablets were added to a Vector 0.5-L coating pan. The tablets were pan coated to achieve 7% target weight gain. Six coated tablets were encapsulated into a size #00 hard gelatin capsule for the dog PK study. Each dog was dosed with 2 capsules (12 coated tablets).

Air volume: 50 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 12
Composition of Enteric Coated Tablet

| Ingredient | % (w/w) | Batch Amt. (g) |
|---|---|---|
| Compound II seal coated tablets | 93.58 | 306 |
| and placebo tablets* | | |
| Acryl-EZE II 493Z180022 | 6.52 | 21 |
| Total | 100.00 | 327 |

### Example 6—Preparation of Compound (II) Enteric Coated Tablets of 6.67 mg Strength for 20 g Dose with Reduced SNAC (3 Tablets Total)

The composition of uncoated core tablets is shown in Table 13. Compound (II), SNAC, and nicotinamide were combined and blended using a Turbula mixer (46 RPM, 10 min). Magnesium stearate was added and the mixture was blended for an additional 5 minutes. The final mixture was compressed using a 9/32″ round standard concave tooling at 600 lb compression force to form tablets at a target weight of 142.7 mg.

TABLE 13
Composition of Uncoated Core Tablets

| | | | |
|---|---|---|---|
| Ingredient | % (w/w) | Batch Amt. (g) | Dose Amt. (mg) |

Intra-granular Components

| Compound (II) | 4.67 | 0.28 | (0.315*) | 20 | (22.47*) |
|---|---|---|---|---|---|
| Salcaprozate sodium | 70.1 | 4.206 | | 300 | |
| Nicotinamide | 23.36 | 1.402 | (1.367**) | 100 | (97.53**) |

| | | | |
|---|---|---|---|
| Magnesium stearate | 1.87 | 0.112 | 8 |
| Total | 100 | 6 | 428 |

*Amount of compound (II) is adjusted based on Assay “as is” of 89%.
**Amount of nicotinamide is adjusted to compensate for potency correction.

The composition of the seal coated tablet is shown in Table 14. Clear An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 45 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 14
Composition of Seal Coated Tablet

| Ingredient | % (w/w) | Batch Amt. (g) |
|---|---|---|
| Uncoated core tablets and placebo tablets* | 98.04 | 300 |
| Opadry 03K19229 | 1.96 | 6 |
| Total | 100.00 | 306 |

*Placebo tablets are added to achieve a suitable batch size for coating process.

The composition of the enteric coated tablet is shown in Table 15. An aqueous suspension of Acryl EZE II was prepared at 10% solid content. Compound (II) seal coated tablets and placebo tablets were added to a Vector 0.5-L coating pan. The tablets were pan coated to achieve 7% target weight gain. Three coated tablets were encapsulated into a size #00 hard gelatin capsule for the dog PK study. Each dog was dosed with 1 capsule (3 coated tablets).

Air volume: 45 cfm; Spray rate: 5 g/min; Atomizing air pressure: 10.8 psi; Pattern air pressure: 10.8 psi; Inlet temperature: 55° C.; Outlet temperature: 37° C.; Pan speed: 20-25 rpm.

TABLE 15
Composition of Enteric Coated Tablet

| Ingredient | % (w/w) | Batch Amt. (g) |
|---|---|---|
| Compound II seal coated tablets | 93.58 | 306 |
| and placebo tablets* | | |
| Acryl-EZE II 493Z180022 | 6.52 | 21 |
| Total | 100.00 | 327 |

*Placebo tablets are added to achieve a suitable batch size for coating process.

### Example 7—Determination of Oral Absorption of Prepared Formulations of Compound (II) in Dogs

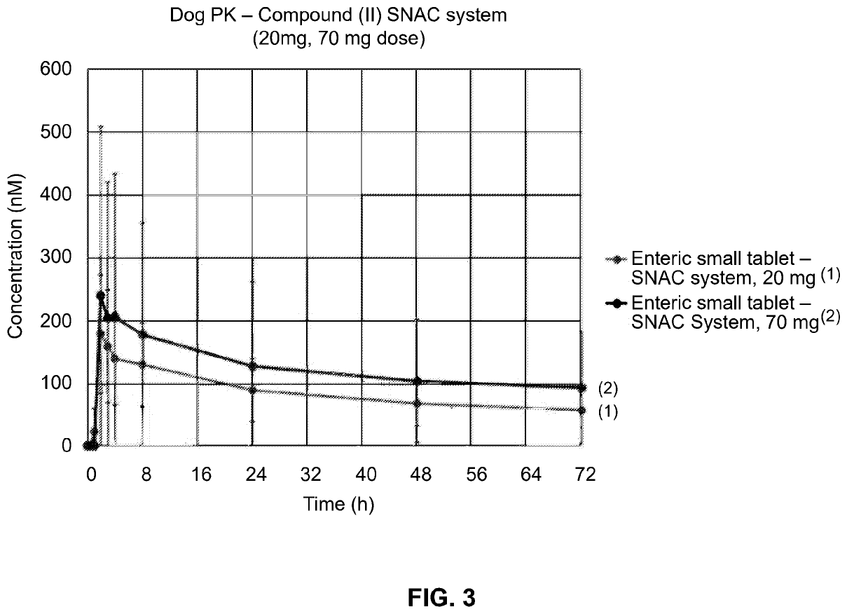

Fasted pentagastrin-pretreated male beagle dogs (n=4) were dosed by oral route followed by gavage flush with water to facilitate gastro-intestinal dissolution. Blood samples were taken at predetermined times over 72 hours post-dose to measure comparative pharmacokinetics between the formulations. Due to the long half-life these were conducted in a parallel group design in different sets of dogs. As shown in FIG. 3 and Tables 16A and 16B, all three formulations provided good concentration of Compound (II) over an extended period of time.

TABLE 16A
Pharmacokinetic Profile of Formulated Tablets

| | | Cmax (nM) | | | AUC₀₋ₜ (nM · h)* | | BA₀₋ₜ (%) | | | AUC_{inf} (nM · h) | | BA_{inf} (%) | | CV |
|---|---|---|---|---|---|---|---|---|---|---|---|---|---|---|
| | Dose | | Std | Tmax (h) | | Std | | Std | CV | | Std | | Std | (inf) |
| | (mg) | Mean | Dev | (Median) | Mean | Dev | Mean | Dev | (%) | Mean | Dev | Mean | Dev | (%) |
| 10 mg IV | 10 | 8546.97 | 638.80 | 0.2 | 322273.57 | 42121.92 | 100.00 | n/a | 13.07 | 402264 | 38879.14 | 100 | n/a | 9.67 |
| Control | | | | | | | | | | | | | | |
| Example 3 | 20 | 178.55 | 94.42 | 2.0 | 6071.75 | 3219.66 | 0.94 | 0.50 | 53.03 | 11660.2 | 5763.76 | 1.45 | 0.72 | 49.43 |
| Example 5 | 20 | 106.73 | 40.84 | 3.5 | 4380.08 | 1666.64 | 0.68 | 0.26 | 38.05 | 18594.33 | 12818.02 | 2.31 | 1.60 | 68.94 |
| Example 6 | 20 | 93.58 | 69.87 | 2.5 | 3676.30 | 2762.62 | 0.57 | 0.43 | 75.15 | 7262.66 | 5074.13 | 0.90 | 0.63 | 69.87 |

TABLE 16B
Pharmacokinetic Profile of Formulated Tablets

| | | | AUC_{0-24 h} | | BA_{0-24 h} | | Relative | |
|---|---|---|---|---|---|---|---|---|
| | | | (nM · h) | | (%) | | BA_{0-72 h} (%) | |
| | Dose | | | Std | | Std | | Std |
| | (mg | % Extrapolated | Mean | Dev | Mean | Dev | Mean | Dev |
| 10 mg IV | 10 | 20.062 | 126167.2 | 12729.57 | 100 | n/a | n/a | n/a |
| Control | | | | | | | | |
| Example 3 | 20 | 48.19 | 2705.75 | 1455.01 | 1.072288 | 0.58 | 100 | n/a |
| Example 5 | 20 | 66.01 | 1816.28 | 621.87 | 0.719792 | 0.25 | 72.14 | 27.45 |
| Example 6 | 20 | 50.72 | 1525.3 | 1172.53 | 0.604476 | 0.46 | 60.55 | 45.50 |

### Example 8—Preparation of Compound (II) Enteric Coated Tablets of 11.67 mg Strength for 70 mg Dose (6 Tablets Total)

The composition of uncoated core tablets is shown in Table 17. Compound (II), SNAC, and nicotinamide were combined and blended using a Turbula mixer (46 RPM, 10 minutes). Magnesium stearate was added and the mixture was blended for an additional 5 minutes. The final mixture was compressed using a 9/32″ round standard concave tooling at 700 lb compression force to form tablets at a target weight of 147.5 mg.

TABLE 17
Composition of Uncoated Core Tablets

| Ingredient | % (w/w) | Batch Amt. (g) | | Dose Amt. (mg) | |
|---|---|---|---|---|---|
| Compound (II) | 7.91 | 0.791 | (0.889*) | 70 | (78.65*) |
| Salcaprozate sodium | 67.8 | 6.78 | | 600 | |
| Nicotinamide | 22.6 | 2.26 | (2.162**) | 200 | (191.39**) |

| | | | |
|---|---|---|---|
| Magnesium stearate | 1.69 | 0.169 | 14.96 |
| Total | 100 | 10 | 885 |

*Amount of compound (II) is adjusted based on Assay “as is” of 89%.
**Amount of nicotinamide is adjusted to compensate for potency correction.

The composition of the seal coated tablet is shown in Table 18. An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 45 cfm; Spray rate: 5 g/min; Atomizing air pressure: 12.1 psi; Pattern air pressure: 10.8 psi; Inlet temperature: 55° C.; Outlet temperature: 40° C.; Pan speed: 23 rpm.

TABLE 18
Composition of Seal Coated Tablet

| Ingredient | % (w/w) | Batch Amt. (g) |
|---|---|---|
| Uncoated core tablets and | 98.04 | 300 |
| placebo tablets* | | |
| Opadry 03K19229 | 1.96 | 6 |
| Total | 100.00 | 306 |

*Placebo tablets are added to achieve a suitable batch size for coating process.

The composition of the enteric coated tablet is shown in Table 19. An aqueous suspension of Acryl EZE II was prepared at 10% solid content. Compound (II) seal coated tablets and placebo tablets were added to a Vector 0.5-L coating pan. The tablets were pan coated to achieve 7% target weight gain. Three coated tablets were encapsulated into a size #00 hard gelatin capsule for the dog PK study. Each dog was dosed with 2 capsules (6 coated tablets).

Air volume: 45 cfm; Spray rate: 5 g/min; Atomizing air pressure: 12.1 psi; Pattern air pressure: 10.8 psi; Inlet temperature: 55° C.; Outlet temperature: 40° C.; Pan speed: 28 rpm.

TABLE 19
Composition of Enteric Coated Tablet

| Ingredient | % (w/w) | Batch Amt. (g) |
|---|---|---|
| Compound II seal coated | 93.58 | 306 |
| tablets and placebo tablets* | | |
| Acryl-EZE II 493Z180022 | 6.52 | 21 |
| Total | 100.00 | 327 |

*Placebo tablets are added to achieve a suitable batch size for coating process.

### Example 9—Preparation of Compound (II) Enteric Coated Tablets of 5.83 mg Strength for for 70 mg Dose (12 Tablets Total)

The composition of uncoated core tablets is shown in Table 20. Compound (II), SNAC, and nicotinamide were combined and blended using a Turbula mixer (46 RPM, 10 min). Magnesium stearate was added and the mixture was blended for an additional 5 minutes. The final mixture was compressed using a 7/32″ round standard concave tooling at 500 lb compression force to form tablets at a target weight of 73.6 mg.

TABLE 20
Composition of Uncoated Core Tablets

| Ingredient | % (w/w) | Batch Amt. (g) | | Dose Amt. (mg) | |
|---|---|---|---|---|---|
| Compound (II) | 7.91 | 0.791 | (0.889*) | 70 | (78.65*) |
| Salcaprozate sodium | 67.8 | 6.78 | | 600 | |
| Nicotinamide | 22.6 | 2.26 | (2.162**) | 200 | (191.39**) |

| | | | |
|---|---|---|---|
| Magnesium stearate | 1.69 | 0.169 | 14.96 |
| Total | 100 | 10 | 885 |

*Amount of compound (II) is adjusted based on Assay “as is” of 89%.
**Amount of nicotinamide is adjusted to compensate for potency correction.

The composition of the seal coated tablet is shown in Table 21. An aqueous suspension of Opadry 03K was prepared at 7.5% solid content. Uncoated core tablets and placebo tablets were added to a Vector 0.5-L coating pan. The tablets were pan coated to achieve 2% target weight gain.

Air volume: 45 cfm; Spray rate: 5 g/min; Atomizing air pressure: 11.0 psi; Pattern air pressure: 11.1 psi; Inlet temperature: 55° C.; Outlet temperature: 40° C.; Pan speed: 20 rpm.

TABLE 21
Composition of Seal Coated Tablet

| Ingredient | % (w/w) | Batch Amt. (g) |
|---|---|---|
| Uncoated core tablets and | 98.04 | 300 |
| placebo tablets* | | |
| Opadry 03K19229 | 1.96 | 6 |
| Total | 100.00 | 306 |

*Placebo tablets are added to achieve a suitable batch size for coating process.

The composition of the enteric coated tablet is shown in Table 22. An aqueous suspension of Acryl EZE II was prepared at 10% solid content. Compound (II) seal coated tablets and placebo tablets were added to a Vector 0.5-L coating pan. The tablets were pan coated to achieve 7% target weight gain. Three coated tablets were encapsulated into a size #00 hard gelatin capsule for the dog PK study. Each dog was dosed with 2 capsules (12 coated tablets).

Air volume: 45 cfm; Spray rate: 5 g/min; Atomizing air pressure: 12.1 psi; Pattern air pressure: 12.0 psi; Inlet temperature: 55° C.; Outlet temperature: 40° C.; Pan speed: 25 rpm.

TABLE 22
Composition of Enteric Coated Tablet

| Ingredient | % (w/w) | Batch Amt. (g) |
|---|---|---|
| Compound II seal coated | 93.58 | 306 |
| tablets and placebo tablets* | | |
| Acryl-EZE II 493Z180022 | 6.52 | 21 |
| Total | 100.00 | 327 |

*Placebo tablets are added to achieve a suitable batch size for coating process.

### Example 10—Determination of Oral Absorption of Prepared Formulations of Compound (II) in Dogs

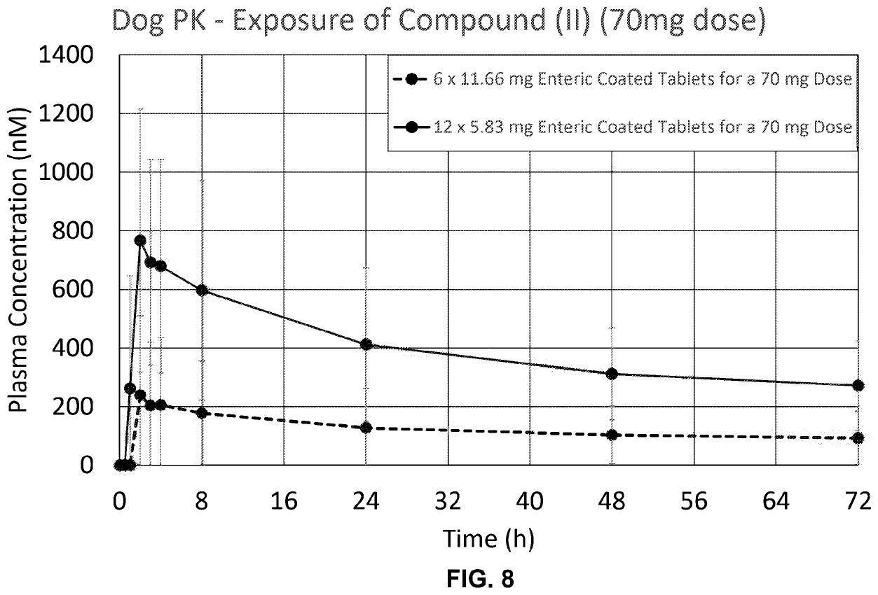

Fasted pentagastrin-pretreated male beagle dogs (n=4) were dosed by oral route followed by gavage flush with water to facilitate gastro-intestinal dissolution. Blood samples were taken at predetermined times over 72 hours post-dose to measure comparative pharmacokinetics between the formulations. Due to the long half-life these were conducted in a parallel group design in different sets of dogs. As shown in FIG. 8 and Tables 23, both formulations provided good concentration of Compound (II) over an extended period of time.

TABLE 23
Pharmacokinetic Profile of Formulated Tablets

| | | Cmax (nM) | | | AUC_{0-72 h} (nM · h) | | |
|---|---|---|---|---|---|---|---|
| | Dose | | Std | Tmax (h) | | Std | BA_{0-72 h} (%) |
| | (mg) | Mean | Dev | (Median) | Mean | Dev | Mean |
| 10 mg IV | 10 | 8546.97 | 638.80 | 0.2 | n/a | n/a | 100.00 |
| Control | | | | | | | |
| Example 6 | 70 | 243.48 | 268.11 | 3.0 | 8875.46 | 8961.11 | 0.39 |
| Example 7 | 70 | 787.75 | 435.29 | 2.0 | 28304.75 | 16253.93 | 1.25 |

| | BA_{0-72 h} (%) | AUC_{inf} (nM · h) | | BA_{inf} (%) | | CV |
|---|---|---|---|---|---|---|
| | Std | | Std | | Std | (inf) |
| | Dev | Mean | Dev | Mean | Dev | (%) |
| 10 mg IV | n/a | 402264 | 38879.14 | 100 | n/a | 9.67 |
| Control | | | | | | |
| Example 6 | 0.40 | 17867.92 | 15378.56 | 0.634548 | 0.546143 | 86.06803 |
| Example 7 | 0.72 | 56253.41 | 27581.54 | 1.997743 | 0.979511 | 49.03087 |

### Example 11—Preparation of Compound (II) Enteric Coated Tablets of 50 mg Strength for 100 mg Dose (2 Tablets Total)

The composition of uncoated core tablets is shown in Table 24. Compound (II), SNAC, nicotinamide, croscarmellose sodium, silicon dioxide, and microcrystalline cellulose were combined and blended using a bin blender at 25 RPM for 250 revolutions. The blended material was passed through a co-mill equipped with 32R screen, and blended again at 25 RPM for 250 revolutions. Magnesium stearate was added to the mixture and blended at 25 RPM for an additional 125 revolutions to form pre-blend. The pre-blend was roller compacted and milled. The milled granules and extra-granular croscarmellose sodium were blended at 25 RPM for 250 revolutions. Extra-granular magnesium stearate was added and blended at 25 RPM for 125 revolutions to form final blend. The final blend was compressed to form tablets at a target weight of 500 mg and hardness of 20 SCU using 0.6030″×0.3140′ (15.3 mm×8 mm) oval standard concave tooling

TABLE 24
Composition of Uncoated Core Tablets

| | | |
|---|---|---|
| Ingredient | % (w/w) | Batch Amt. (g) |

Intragranular Components

| | | |
|---|---|---|
| Compound (II)* | 10 | 120 |
| Salcaprozate sodium | 60 | 720 |
| Nicotinamide | 20 | 240 |
| Microcrystalline cellulose** | 2 | 24 |
| Croscarmellose Sodium | 2 | 24 |
| Silicon dioxide | 2 | 24 |
| Magnesium stearate | 1 | 12 |

Extra-granular Components

| | | |
|---|---|---|
| Croscarmellose Sodium | 2 | 24 |
| Magnesium Stearate | 1 | 12 |
| Total | 100 | 1,200 |

*Amount of compound (II) is adjusted based on Assay “as is”.
**Amount of microcrystalline cellulose is adjusted to compensate for potency correction.

TABLE 25
Amount of ingredients per tablet and per dose
for 2 × 50 mg tablet to achieve 100 mg dose

| | % | Amount per | Amount per |
|---|---|---|---|
| Ingredient | (w/w) | Tablet (mg) | Dose (mg) |
| Compound (II)* | 10 | 50 | 100 |
| Salcaprozate sodium | 60 | 300 | 600 |
| Nicotinamide | 20 | 100 | 200 |
| Microcrystalline cellulose** | 2 | 10 | 20 |
| Croscarmellose Sodium | 4 | 20 | 40 |
| Silicon dioxide | 2 | 10 | 20 |
| Magnesium stearate | 2 | 10 | 20 |
| Total | 100 | 500 | 1,000 |

*Amount of compound (II) is adjusted based on Assay “as is”.
**Amount of microcrystalline cellulose is adjusted to compensate for potency correction.

The tablets were coated in a 4-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Each dog was dosed with 2 tablets (Table 25).

### Example 12—Preparation of Compound (II) Enteric Coated Tablets of 10 mg Strength for Dose of 100 mg (10 Tablets Total)

The final blend from the above example was compressed to form tablets at a target weight of 100 mg and target harness of 10 SCU using 7/32″ (5.55 mm) round standard concave tooling.

TABLE 26
Amount of ingredients per tablet and per dose
for 10 × 10 mg tablet to achieve 100 mg dose

| | % | Amount per | Amount per |
|---|---|---|---|
| Ingredient | (w/w) | Tablet (mg) | Dose (mg) |
| Compound (II)* | 10 | 10 | 100 |
| Salcaprozate sodium | 60 | 60 | 600 |
| Nicotinamide | 20 | 20 | 200 |
| Microcrystalline cellulose** | 2 | 2 | 20 |
| Croscarmellose Sodium | 4 | 4 | 40 |
| Silicon dioxide | 2 | 2 | 20 |
| Magnesium stearate | 2 | 2 | 20 |
| Total | 100 | 100 | 1,000 |

*Amount of compound (II) is adjusted based on Assay “as is”.
**Amount of microcrystalline cellulose is adjusted to compensate for potency correction.

The tablets were coated in a 4-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Five enteric coated tablets were encapsulated into a size #00 hard gelatin capsule for the dog PK study. Each dog was dosed with 2 capsules (10 enteric coated tablets, Table 26).

### Example 13—Preparation of Compound (II) Enteric Tablets of 2.5 mg Strength for 100 mg Dose (40 Tablets Total)

The composition of uncoated core tablets is shown in Table 27. Compound (II), SNAC, nicotinamide, croscarmellose sodium, silicon dioxide, and microcrystalline cellulose were combined and blended using a bin blender at 25 RPM for 250 revolutions. The blended material was passed through a co-mill equipped with 32R screen, and blended again at 25 RPM 250 revolutions. Magnesium stearate was added to the mixture and blended at 25 RPM for an additional 125 revolutions to form pre-blend. The pre-blend was roller compacted and milled. The milled granules and extra-granular croscarmellose sodium were blended at 25 RPM for 250 revolutions. Extra-granular magnesium stearate was added and blended at 25 RPM for 125 revolutions to form final blend. The final blend was compressed to form tablets at a target weight of 25 mg and target hardness of 5 SCU using ⅛″ (3.175 mm) multitip (7 tips) round standard concave tooling.

TABLE 27
Composition of Uncoated Core Tablets

| | | |
|---|---|---|
| Ingredient | % (w/w) | Batch Amt. (g) |

Intragranular Components

| | | |
|---|---|---|
| Compound (II)* | 10 | 200 |
| Salcaprozate sodium | 60 | 1,200 |
| Nicotinamide | 20 | 400 |
| Microcrystalline cellulose** | 2 | 40 |
| Croscarmellose Sodium | 2 | 40 |
| Silicon dioxide | 2 | 40 |
| Magnesium stearate | 1 | 20 |

Extra-granular Components

| | | |
|---|---|---|
| Croscarmellose Sodium | 2 | 40 |
| Magnesium Stearate | 1 | 20 |
| Total | 100 | 2,000 |

*Amount of compound (II) is adjusted based on Assay “as is”.
**Amount of microcrystalline cellulose is adjusted to compensate for potency correction.

TABLE 28
Amount of Ingredients per Tablet and per Dose
for 40 × 2.5 mg Tablet to Achieve 100 mg Dose

| | % | Amount per | Amount per |
|---|---|---|---|
| Ingredient | (w/w) | Tablet (mg) | Dose (mg) |
| Compound (II)* | 10 | 2.5 | 100 |
| Salcaprozate sodium | 60 | 15 | 600 |
| Nicotinamide | 20 | 5 | 200 |
| Microcrystalline cellulose** | 2 | 0.5 | 20 |
| Croscarmellose Sodium | 4 | 0.1 | 40 |
| Silicon dioxide | 2 | 0.05 | 20 |
| Magnesium stearate | 2 | 0.05 | 20 |
| Total | 100 | 25 | 1,000 |

*Amount of compound (II) is adjusted based on Assay “as is”.
**Amount of microcrystalline cellulose is adjusted to compensate for potency correction.

The tablets were coated in a 4-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 10% target weight gain. Twenty coated tablets were encapsulated into a size #00 hard gelatin capsule for the dog PK study. Each dog was dosed with 2 capsules (40 coated tablets, Table 28).

### Example 14—Preparation of Matching Placebo for Compound (II) Enteric Tablets for 50 mg Dose with 5×10 mg Tablets and 5× Placebo Tablets

The composition of uncoated core tablets is shown in Table 29. SNAC, nicotinamide, croscarmellose sodium, silicon dioxide, and microcrystalline cellulose were combined and blended using using a mini V-blender at 28 RPM for 10 minutes. The blended material was passed through a 20 mesh screen, and blended again at 28 RPM for 10 minutes. Magnesium stearate was added to the mixture and blended at 28 RPM for an additional 5 minutes to form pre-blend. The pre-blend was compressed to form compacts at target weight of 400 mg and target solid fraction of 0.7 using 11.28 mm round flat faced tooling. The compacted were milled using oscillator equipped with 4-millimeter and 1-millimeter screen. The milled granules and extra-granular croscarmellose sodium were blended at 28 RPM for 10 minutes. Extra-granular magnesium stearate was added and blended at 28 RPM for 10 minutes to form final blend. The final blend was compressed to form tablets at a target weight of 100 mg and a target hardness of 10 SCU using 7/32″ (5.55 mm) round standard concave tooling.

TABLE 29
Composition of Uncoated Core Tablets

| | | |
|---|---|---|
| Ingredient | % (w/w) | Batch Amt. (g) |

Intragranular Components

| | | |
|---|---|---|
| Compound (II) | N/A | N/A |
| Salcaprozate sodium | 60 | 30 |
| Nicotinamide | 20 | 10 |
| Microcrystalline cellulose | 12 | 6 |
| Croscarmellose Sodium | 2 | 1 |
| Silicon dioxide | 2 | 1 |
| Magnesium stearate | 1 | 0.5 |

Extra-granular Components

| | | |
|---|---|---|
| Croscarmellose Sodium | 2 | 1 |
| Magnesium Stearate | 1 | 0.5 |
| Total | 100 | 50 |

TABLE 30
Amount of ingredients per tablet and
per dose for 5 × placebo tablets

| | % | Amount per | Amount per |
|---|---|---|---|
| Ingredient | (w/w) | Tablet (mg) | Dose (mg) |
| Compound (II) | N/A | N/A | N/A |
| Salcaprozate sodium | 60 | 60 | 300 |
| Nicotinamide | 20 | 20 | 100 |
| Microcrystalline cellulose | 12 | 12 | 60 |
| Croscarmellose Sodium | 4 | 4 | 20 |
| Silicon dioxide | 2 | 2 | 10 |
| Magnesium stearate | 2 | 2 | 10 |
| Total | 100 | 100 | 500 |

The tablets were coated in a 4-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Five enteric coated tablets were encapsulated into a size #00 hard gelatin capsule for the dog PK study. Each dog was dosed with 1 capsule of active tablets and 1 capsule of matching placebo (Table 30).

### Example 15—Preparation of Compound (II) Enteric Coated Tablets with 5 mg Strength for 50 mg Dose (10 Tablets Total)

The composition of uncoated core tablets is shown in Table 31. Compound (II), SNAC, nicotinamide, croscarmellose sodium, silicon dioxide, and microcrystalline cellulose were combined and blended using using a mini V-blender at 28 RPM for 10 minutes. The blended material was passed through a 20 mesh screen, and blended again at 28 RPM for 10 minutes. Magnesium stearate was added to the mixture and blended at 28 RPM for an additional 5 minutes to form pre-blend. The pre-blend was compressed to form compacts at target weight of 400 mg and target solid fraction of 0.7 using 11.28 mm round flat faced tooling. The compacted were milled using oscillator equipped with 4-millimeter and 1-millimeter screen. The milled granules and extra-granular croscarmellose sodium were blended at 28 RPM for 10 minutes. Extra-granular magnesium stearate was added and blended at 28 RPM for 10 minutes to form final blend. The final blend was compressed to form tablets at a target weight of 100 mg and a target hardness of 10 SCU using 7/32″ (5.55 mm) round standard concave tooling.

TABLE 31
Composition of Uncoated Core Tablets

| | | |
|---|---|---|
| Ingredient | % (w/w) | Batch Amt. (g) |

Intragranular Components

| | | |
|---|---|---|
| Compound (II)* | 5 | 2.5 |
| Salcaprozate sodium | 60 | 30 |
| Nicotinamide | 20 | 10 |
| Microcrystalline cellulose** | 7 | 3.5 |
| Croscarmellose Sodium | 2 | 1 |
| Silicon dioxide | 2 | 1 |
| Magnesium stearate | 1 | 0.5 |

Extra-granular Components

| | | |
|---|---|---|
| Croscarmellose Sodium | 2 | 1 |
| Magnesium Stearate | 1 | 0.5 |
| Total | 100 | 50 |

*Amount of compound (II) is adjusted based on Assay “as is”.
**Amount of microcrystalline cellulose is adjusted to compensate for potency correction.

TABLE 32
Amount of ingredients per tablet and per dose
for 10 × 5 mg tablet to achieve 50 mg dose

| | % | Amount per | Amount per |
|---|---|---|---|
| Ingredient | (w/w) | Tablet (mg) | Dose (mg) |
| Compound (II) | 5 | 5 | 50 |
| Salcaprozate sodium | 60 | 60 | 600 |
| Nicotinamide | 20 | 20 | 200 |
| Microcrystalline cellulose | 7 | 7 | 70 |
| Croscarmellose Sodium | 4 | 4 | 40 |
| Silicon dioxide | 2 | 2 | 20 |
| Magnesium stearate | 2 | 2 | 20 |
| Total | 100 | 100 | 1,000 |

The tablets were coated in a 4-L pan LDCS Hi-Coater with 2 layers of coating. The first layer was seal coating with Opadry 03K aqueous suspension (10% w/w solid content) to achieve 2% target weight gain. The second layer was enteric coating with Acryl-EZE II aqueous suspension (20% w/w solid content) to achieve 7% target weight gain. Five enteric coated tablets were encapsulated into a size #00 hard gelatin capsule for the dog PK study. Each dog was dosed with 2 capsule (10 enteric coated tablets, Table 32).

### Example 16—Determination of Oral Absorption of Prepared Formulations of Compound (II) in Dogs

Dogs were administered formulations as follows:
2×50 mg tablets with 10% API (Example 10, 88.6 mg dose after API potency correction)
10×10-mg tablets with 10% API (Example 11, 88.6 mg dose after API potency correction)
5×10 mg tablets with 10% API (Example 11, 44.3 mg dose after API potency correction) and 5 placebo tablets with enhancer only.
10×5 mg tablets with 5% API (44.3 mg dose after API potency correction)
40×2.5 mg tablets with 10% API (Example X, 100 mg dose after potency correction)

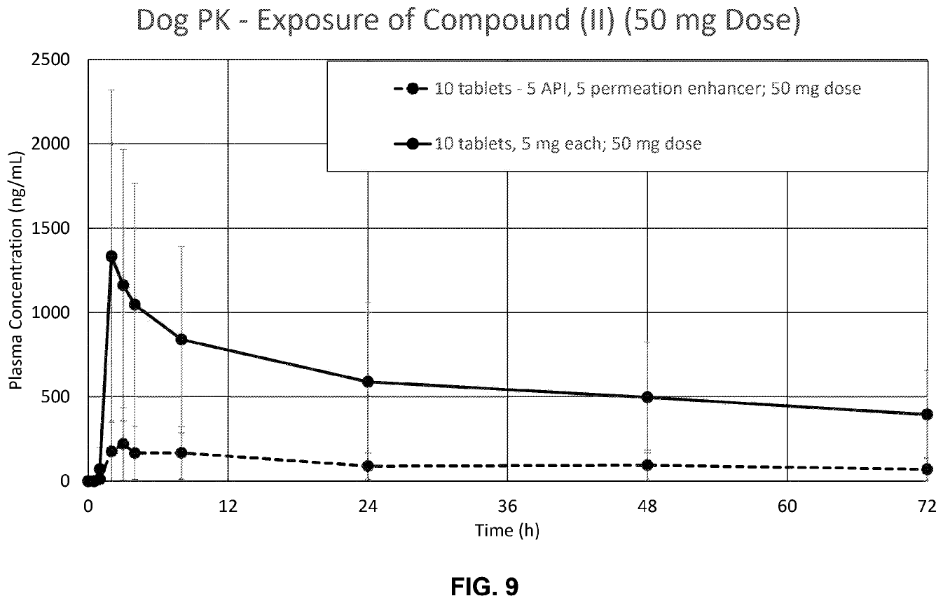

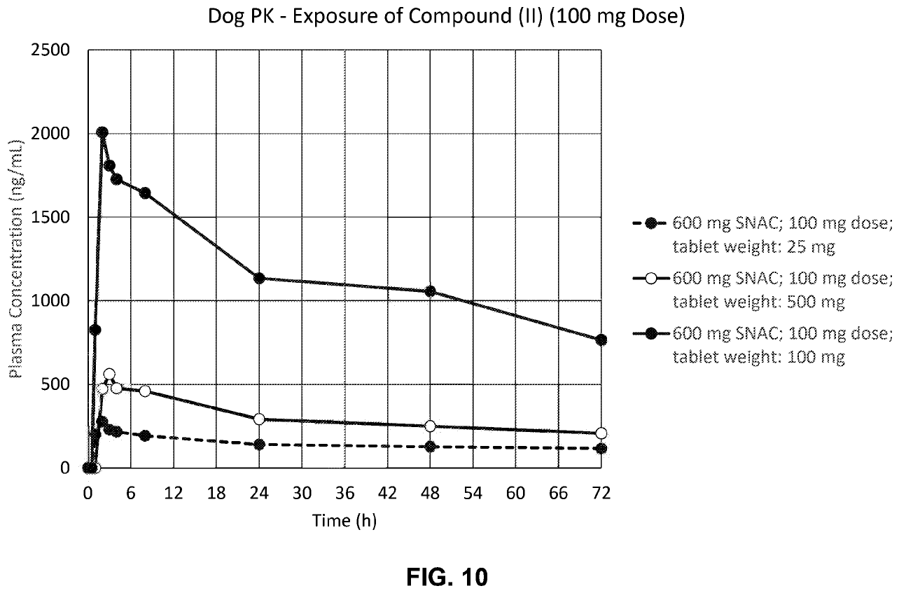

As shown in Table 33 and in FIGS. 9 and 10, dosing dogs with 10×10 mg tablets provided higher bioavailability compared to dosing with 2×50 mg tablets. Without being bound by theory, this may be a result of higher amounts of the permeation enhancer accessible due to the increased surface area. Interestingly, however, further increasing the number of tablets to 40 (and lowering the dose to X per tablet) provided lower bioavailability. Without being bound by theory, this may be due to an insufficient local concentration of salcaprozate sodium for permeation enhancement.

TABLE 33
Pharmacokinetic Data

| | | Cmax (ng/mL) | | | AUC_{0-24 h} (ng · h/mL) | | BA_{0-24 h} (%) | | | AUC_{0-72 h} (ng · h/mL) | | BA_{0-72 h} (%) | | |
|---|---|---|---|---|---|---|---|---|---|---|---|---|---|---|
| | Dose | | Std | Tmax (h) | | Std | | Std | CV | | Std | | Std | CV |
| | (mg) | Mean | Dev | (Median) | Mean | Dev | Mean | Dev | (%) | Mean | Dev | Mean | Dev | (%) |
| 10 mg IV | 10 | 25466.29 | 1903.34 | 0.2 | 375888.35 | 37927.63 | 100.00 | n/a | 10.09 | 832979.81 | 115022.86 | 100.00 | n/a | 13.81 |
| Control | | | | | | | | | | | | | | |
| 2 tablets, | 89 | 571.63 | 324.30 | 3.0 | 9151.23 | 5441.70 | 0.27 | 0.16 | 59.46 | 21123.74 | 11402.56 | 0.29 | 0.15 | 59.26 |
| 50 mg each; | | | | | | | | | | | | | | |
| 100 mg dose | | | | | | | | | | | | | | |
| 10 tablets, | 89 | 2151.25 | 1242.09 | 2.5 | 34265.32 | 20322.19 | 1.03 | 0.61 | 59.31 | 82411.89 | 50732.50 | 1.12 | 0.69 | 61.56 |
| 10 mg each; | | | | | | | | | | | | | | |
| 100 mg dose | | | | | | | | | | | | | | |
| 10 tablets - | 44 | 220.86 | 215.86 | 3.0 | 3214.53 | 2959.54 | 0.19 | 0.18 | 92.07 | 7400.88 | 6845.78 | 0.20 | 0.19 | 92.50 |
| 5 API, 5 | | | | | | | | | | | | | | |
| permeation | | | | | | | | | | | | | | |
| enhancer; | | | | | | | | | | | | | | |
| 50 mg dose | | | | | | | | | | | | | | |
| 10 tablets, | 44 | 1360.17 | 964.46 | 2.0 | 18273.64 | 12788.13 | 1.10 | 0.77 | 69.98 | 42012.17 | 29117.08 | 1.14 | 0.79 | 69.31 |
| 5 mg each; | | | | | | | | | | | | | | |
| 50 mg dose | | | | | | | | | | | | | | |
| 40 tablets, | 100 | 327.75 | 198.42 | 2.5 | 4244.27 | 2889.48 | 0.11 | 0.08 | 68.08 | 10363.26 | 7435.92 | 0.12 | 0.09 | 71.75 |
| 2.5 mg each; | | | | | | | | | | | | | | |
| 100 mg dose | | | | | | | | | | | | | | |

### Example 17—Bioavailability of SNAC Formulations

Multiple pharmacokinetic dog studies were conducted with 2-4 arms available for scale-up formulation optimization. Two dose levels at 50 mg and 100 mg were assumed for Phase 1 human studies. Results are shown in Table 34.

TABLE 34
Bioavailability of Various Formulations

| | | | |
|---|---|---|---|
| API Dose (mg) | 50 | 50 | 100 |
| SNAC Dose (mg) | 600 | 600 | 600 |
| Nicotinamide Dose (mg) | 200 | 200 | 200 |
| Number of Tablets | 10 × 5 mg | 5 × 10 mg | 10 × 10 mg |
| | Tablets | Tablets | Tablets |
| | | 5 × True | |
| | | Placebo | |
| Tablet Strength (mg) | 10 | 5 | 10 |
| Tablet Weight (mg) | 100 | 100 | 100 |
| Bioavailability, 0-72 h | 1.14% | 0.20% | 1.12% |

As shown in Table 34, using placebo tablets as a source of SNAC to allow for fewer tablets containing API provided significantly lower bioavailability than when only tablets with API were dosed. In addition, 50 mg API and 100 mg API produced similar bioavailabilities.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A pharmaceutical composition comprising a biologically active compound, a salcaprozate salt, and nicotinamide.

2. The pharmaceutical composition of claim 1, wherein the salcaprozate salt is salcaprozate sodium.

3. The pharmaceutical composition of claim 1 or 2, further comprising one or more protease inhibitors.

4. The pharmaceutical composition of claim 3, wherein the one or more protease inhibitors comprises one or more trypsin inhibitors.

5. The pharmaceutical composition of claim 4, wherein the one or more trypsin inhibitors are isolated from bovine pancreas, raw avian egg white, soybean, or lima bean.

6. The pharmaceutical composition of any one of claims 3 to 5, wherein the one or more protease inhibitors are selected from soybean trypsin inhibitor, aprotinin, lima bean trypsin inhibitor, ovomucoid trypsin inhibitor, and combinations thereof.

7. The pharmaceutical composition of any one of claims 1 to 6, wherein the biologically active compound comprises a cyclic peptide.

8. The pharmaceutical composition of claim 7, wherein the cyclic peptide comprises from 5 to 30 amino acids.

9. The pharmaceutical composition of claim 7 or 8, wherein the cyclic peptide comprises from 5 to 20 amino acids.

10. The pharmaceutical composition of any one of claims 7 to 9, wherein the cyclic peptide comprises from 12 to 16 amino acids.

11. The pharmaceutical composition of any one of claims 7 to 10, wherein the cyclic peptide is a compound of formula (I): 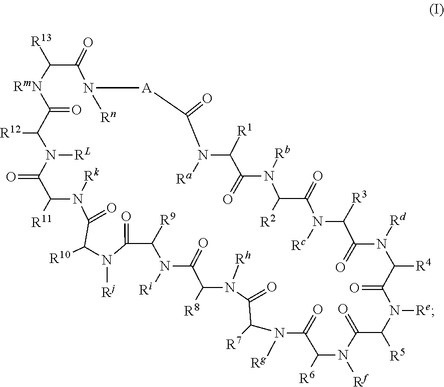
or a pharmaceutically acceptable salt thereof, wherein:
A is selected from: 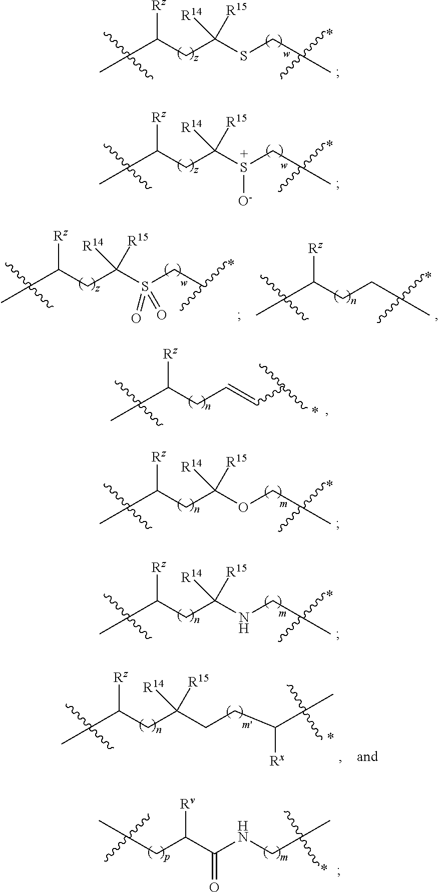
wherein 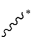
denotes the point of attachment to the carbonyl group and denotes the 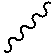
point of attachment to the nitrogen atom;
n is 0, 1, or 2;
m is 1 or 2;
m′ is 0 or 1;
z is 0, 1 or 2;
w is 1 or 2;
p is 0, 1, or 2;
R¹⁴ and R¹⁵ are independently selected from hydrogen and methyl;
R^{x} is selected from hydrogen, amino, hydroxy, and methyl;
R^{v} is hydrogen, methyl, or a natural amino acid side chain;
R^{z} is selected from hydrogen and —C(O)NHR¹⁶;
R¹⁶ is selected from hydrogen, —CHR¹⁷C(O)NH₂, —CHR¹⁷C(O)NHCHR¹⁷C(O)NH₂, —CH₂C(O)NHCH(R¹⁷)C(O)NHCH(R^{17a})C(O)NH₂; —CH₂C(O)NHCH(R¹⁷)CO₂H; and —(C(R^{17a})₂)₂—X′—R³⁰;
each R¹⁷ is independently selected from hydrogen, —CH₃, —CH₂OH, and —(CH₂)_{w}-triazolyl-X—R³⁵;
R³⁵ is selected from —CO₂H and CH₃;
each R^{17a} is independently selected from hydrogen and —CH₂CO₂H;
X′ is a chain of between 8 and 46 atoms wherein the atoms are selected from carbon and oxygen and wherein the chain may contain one, two, or three C(O)NH groups embedded therein;
and wherein the chain is optionally substituted with one or two groups independently selected from —CO₂H, —C(O)NH₂, —CH₂C(O)NH₂, and —CH₂CO₂H;
R³⁰ is selected from —CO₂H, —C(O)NR^{w}R^{x}, and —CH₃ wherein R^{w} and R^{w′} are independently selected from hydrogen and C₁-C₆alkyl, provided that when X′ is all carbon, R³⁰ is other than-CH₃;
X is selected from
—(CH₂)₂CH(CO₂H)NHC(O)(CH₂)_{f};
—(CH₂CH₂O)_{g}; and
—(CH₂CH₂O)_{g}CH₂CH₂NHC(O)CH₂CH₂CH(CO₂H)NHC(O)(CH₂)_{f};
f is 14, 15, or 16;
g is 3, 4, 5, 6, 7, 8, 9, 10, or 11;
R¹³ is selected from a natural amino acid, an unnatural amino acid, —(C(R^{17a})₂)₂—X′—R³⁰, and —(CH₂)_{w}-triazolyl-X—R³⁵;
R¹, R², R³, R⁴, R⁵, R⁶, R⁷, R⁸, R⁹, R¹⁰, R¹¹, and R¹² are independently selected from a natural amino acid side chain and an unnatural amino acid side chain or form a ring with the corresponding vicinal R group as described below;
R^{a}, R^{c}, R^{f}, R^{h}, Rⁱ, R^{j}, R^{m}, and Rⁿ are each independently selected from hydrogen and methyl;
R^{b} is hydrogen or methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{d} is hydrogen or methyl, or, R^{d} and R⁴, together with the atoms to which they are attached, can form a ring selected from azetidine, pyrrolidine, morpholine, piperidine, piperazine, and tetrahydrothiazole; wherein each ring is optionally substituted with one to four groups independently selected from amino, cyano, methyl, halo, hydroxy, and phenyl;
R^{e} is hydrogen or methyl, or R^{e} and R⁵, together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{k} is hydrogen or methyl, or, R^{k} and R¹¹, together with the atoms to which they are attached selected from azetidine, pyrrolidine, morpholine, piperidine, piperazine, and tetrahydrothiazole; wherein each ring is optionally substituted with one to four groups independently selected from amino, cyano, methyl, halo, and hydroxy; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

12. The pharmaceutical composition of claim 11, wherein
R^{c}, R^{f}, R^{h}, Rⁱ, R^{m}, and Rⁿ are hydrogen;
R^{b} is methyl, or, R^{b} and R², together with the atoms to which they are attached, form a ring selected from azetidine, pyrrolidine, morpholine, piperidine, piperazine, and tetrahydrothiazole; wherein each ring is optionally substituted with one to four groups independently selected from amino, cyano, methyl, halo, and hydroxy;
R^{g} is hydrogen or methyl or R^{g} and R⁷, together with the atoms to which they are attached, can form a ring selected from azetidine, pyrrolidine, morpholine, piperidine, piperazine, and tetrahydrothiazole; wherein each ring is optionally substituted with one to four groups independently selected from amino, benzyl optionally substituted with a halo group, benzyloxy, cyano, cyclohexyl, methyl, halo, hydroxy, isoquinolinyloxy optionally substituted with a methoxy group, quinolinyloxy optionally substituted with a halo group, and tetrazolyl; and wherein the pyrrolidine and the piperidine ring are optionally fused to a cyclohexyl, phenyl, or indole group; and
R^{L} is methyl or, R^{L} and R¹², together with the atoms to which they are attached, form a ring selected from azetidine and pyrrolidine, wherein each ring is optionally substituted with one to four independently selected from amino, cyano, methyl, halo, and hydroxy.

13. The pharmaceutical composition of claim 11 or 12, wherein
R¹⁶ is —CH₂C(O)NHCH(R¹⁷)CO₂H or —(C(R^{17a})₂)₂—X′—R³⁰; and
R¹⁷ is —(CH₂)_{w}-triazolyl-X—R³⁵.

14. The pharmaceutical composition of any one of claims 7 to 13, wherein the cyclic peptide is a compound of formula (II): 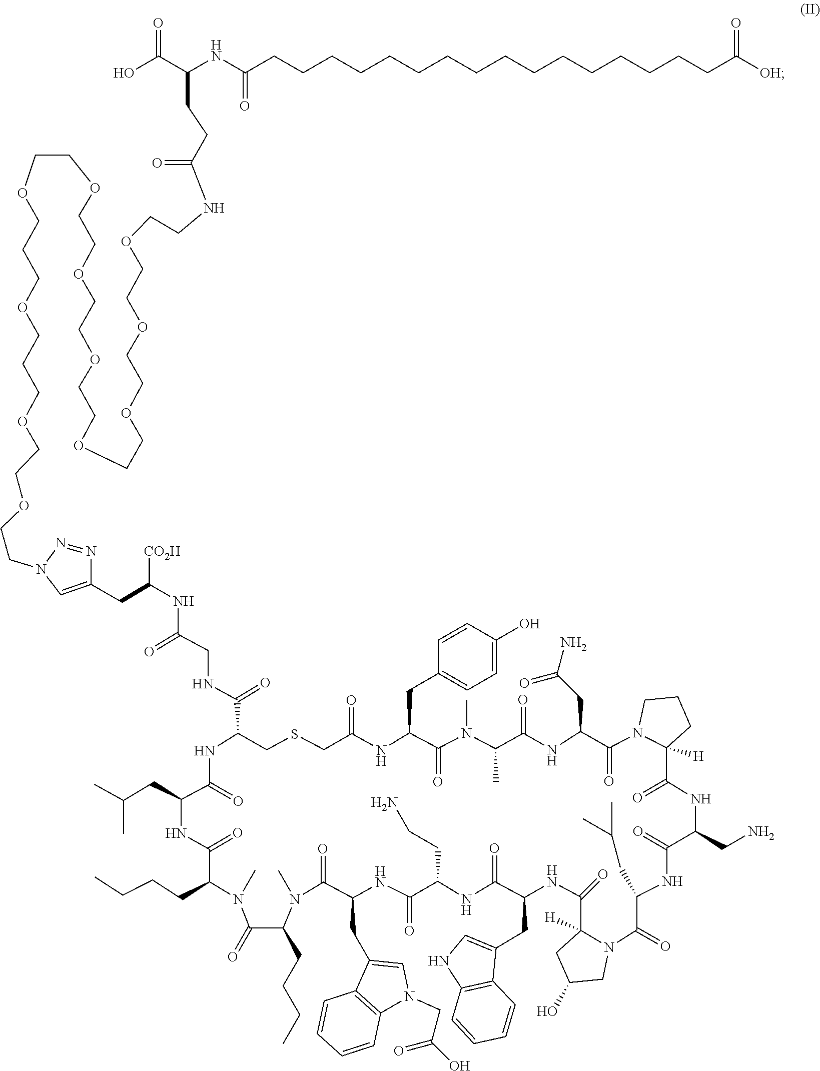 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof.

15. The pharmaceutical composition of any one of claims 1 to 14, wherein the biologically active compound is present in an amount of about 0.1% (w/w) to about 50% (w/w).

16. The pharmaceutical composition of any one of claims 1 to 15, wherein the biologically active compound is present in an amount of about 1% (w/w) to about 45% (w/w).

17. The pharmaceutical composition of any one of claims 1 to 16, wherein the biologically active compound is present in an amount of about 2% (w/w) to about 40% (w/w).

18. The pharmaceutical composition of any one of claims 1 to 16, wherein the biologically active compound is present in an amount of about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 21% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 31% (w/w), about 32% (w/w), about 33% (w/w), about 34% (w/w), about 35% (w/w), about 36% (w/w), about 37% (w/w), about 38% (w/w), about 39% (w/w), about 40% (w/w), about 41% (w/w), about 42% (w/w), about 43% (w/w), about 44% (w/w), or about 45% (w/w).

19. The pharmaceutical composition of any one of claims 1 to 18, wherein the salcaprozate salt is present in an amount of about 30% (w/w) to about 95% (w/w).

20. The pharmaceutical composition of any one of claims 1 to 18, wherein the salcaprozate salt is present in an amount of about 50% (w/w) to about 90% (w/w).

21. The pharmaceutical composition of any one of claims 1 to 18, wherein the salcaprozate salt is present in an amount of about 60% (w/w) to about 85% (w/w).

22. The pharmaceutical composition of any one of claims 1 to 18, wherein the salcaprozate salt is present in an amount of about 60% (w/w) to about 80% (w/w).

23. The pharmaceutical composition of any one of claims 1 to 22, wherein the biologically active compound and the salcaprozate salt are present in a w/w ratio of about 0.02 to about 1.5.

24. The pharmaceutical composition of any one of claims 1 to 22, wherein the biologically active compound and the salcaprozate salt are present in a w/w ratio of about 0.03 to about 1.4.

25. The pharmaceutical composition of any one of claims 1 to 22, wherein the biologically active compound and the salcaprozate salt are present in a w/w ratio of about 0.02, about 0.03, about 0.04, about 0.05, about 0.06, about 0.07, about 0.08, about 0.09, about 0.10, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1.0, about 1.05, about 1.1, about 1.15, about 1.2, about 1.25, about 1.3, about 1.35, about 1.4, about 1.45, or about 1.5.

26. The pharmaceutical composition of any one of claims 1 to 25, wherein the nicotinamide is present in an amount of about 5% (w/w) to about 60% (w/w).

27. The pharmaceutical composition of any one of claims 1 to 25, wherein the nicotinamide is present in an amount of about 10% (w/w) to about 50% (w/w).

28. The pharmaceutical composition of any one of claims 1 to 25, wherein the nicotinamide is present in an amount of about 15% (w/w) to about 40% (w/w).

29. The pharmaceutical composition of any one of claims 1 to 25, wherein the nicotinamide is present in an amount of about 20% (w/w) to about 30% (w/w).

30. The pharmaceutical composition of any one of claims 3 to 29, wherein the one or more protease inhibitors are present in an amount of about 0.1% (w/w) to about 50% (w/w), about 0.5% (w/w) to about 40% (w/w), about 0.75% (w/w) to about 30% (w/w), about 1% (w/w) to about 25% (w/w), or about 5% (w/w) to about 20% (w/w).

31. A pharmaceutical composition comprising:
(a) a cyclic peptide;
(b) salcaprozate sodium at a concentration of about 45% (w/w) to about 80% (w/w); and
(c) nicotinamide at a concentration of about 15% (w/w) to about 30% (w/w).

32. A pharmaceutical composition comprising:
(a) a cyclic peptide;
(b) salcaprozate sodium at a concentration of about 45% (w/w) to about 80% (w/w);
(c) nicotinamide at a concentration of about 15% (w/w) to about 30% (w/w); and
(d) a protease inhibitor at a concentration of about 1% (w/w) and about 20% (w/w).

33. The pharmaceutical composition of claim 31 or 32, wherein the cyclic peptide is a compound of formula (II): 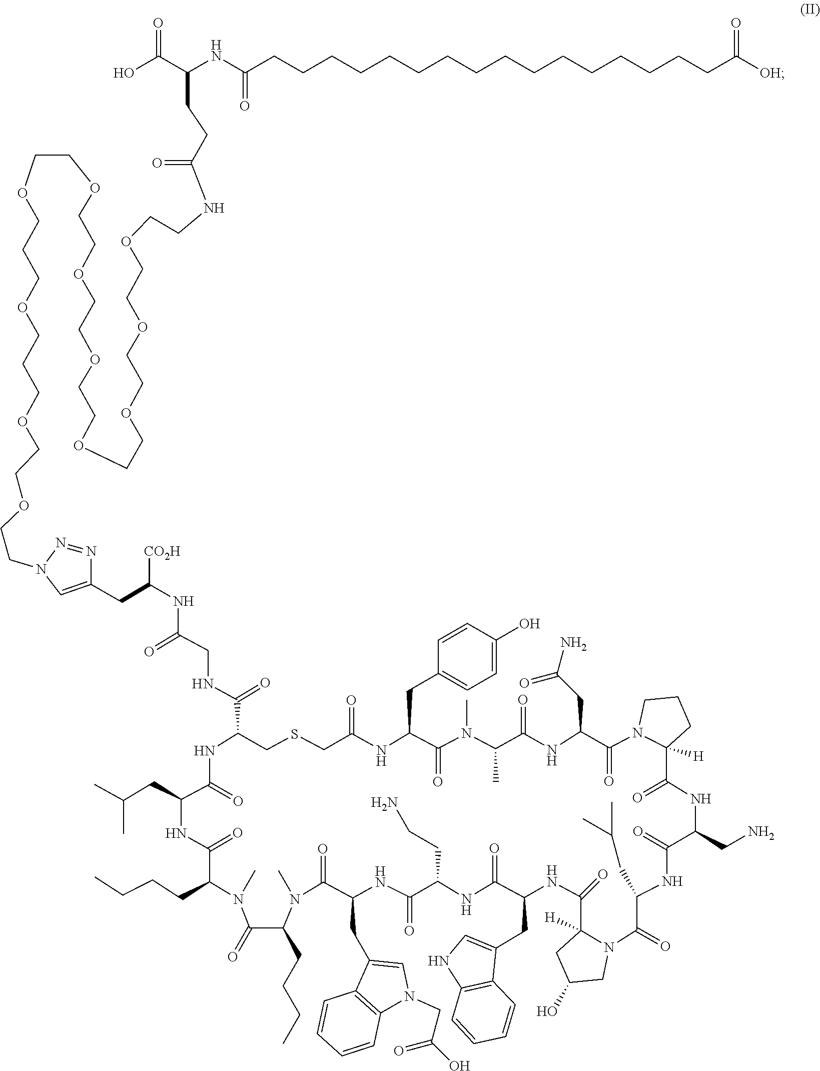 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof.

34. The pharmaceutical composition of claim 33, wherein the cyclic peptide is present in an amount of about 1% (w/w) to about 40% (w/w).

35. A method of improving oral bioavailability of a biologically active compound in a subject in need thereof comprising formulating the biologically active compound with a salcaprozate salt and nicotinamide.

36. The method of claim 35, wherein the formulated biologically active compound has improved oral bioavailability compared to the biologically active compound without the salcaprozate salt and nicotinamide.

37. The method of claim 36, wherein the oral bioavailability is improved at least by about 10%, at least by about 20%, at least by about 30%, at least by about 40%, at least by about 50%, at least by about 60%, at least by about 70%, at least by about 80%, at least by about 90%, at least about 100%, at least about 200%, at least about 300% at least about 400%, at least about 500%, at least about 600%, at least 700%, or at least 800%.

38. The method of any one of claims 35 to 37, wherein the salcaprozate salt is salcaprozate sodium.

39. The method of any one of claims 35 to 38, wherein the biologically active compound is a cyclic peptide.

40. The method of any one of claims 35 to 39, wherein the cyclic peptide is a compound of formula (II): 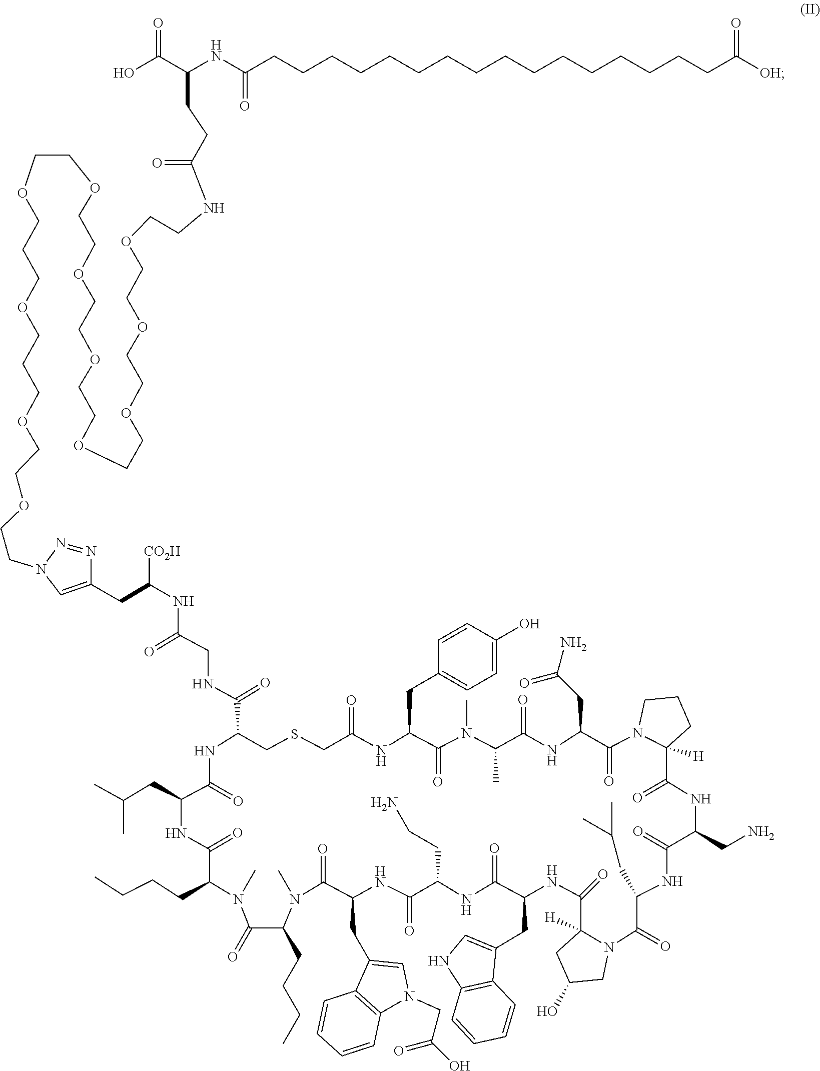 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof.

41. A composition comprising a compound of formula (II): 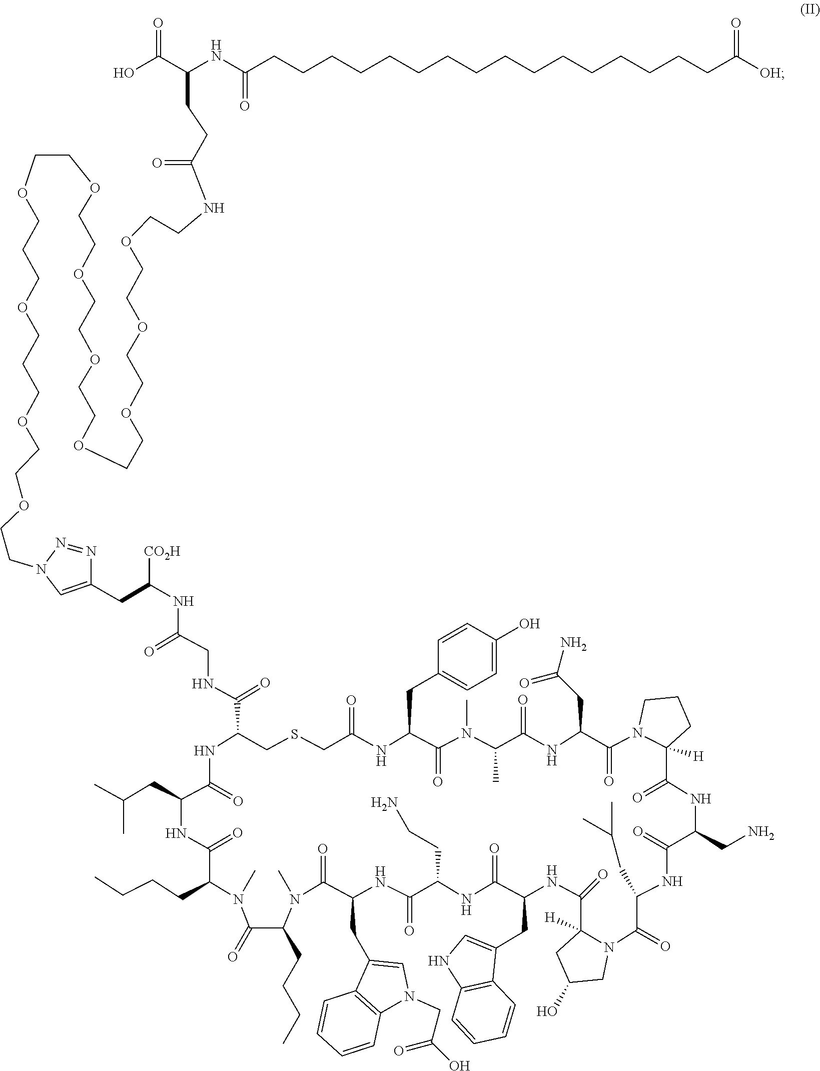 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof;
salcaprozate sodium, and nicotinamide, wherein the salcaprozate sodium and nicotinamide provide an oral bioavailability of >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

42. A method of improving the oral bioavailability of a compound of formula (II): 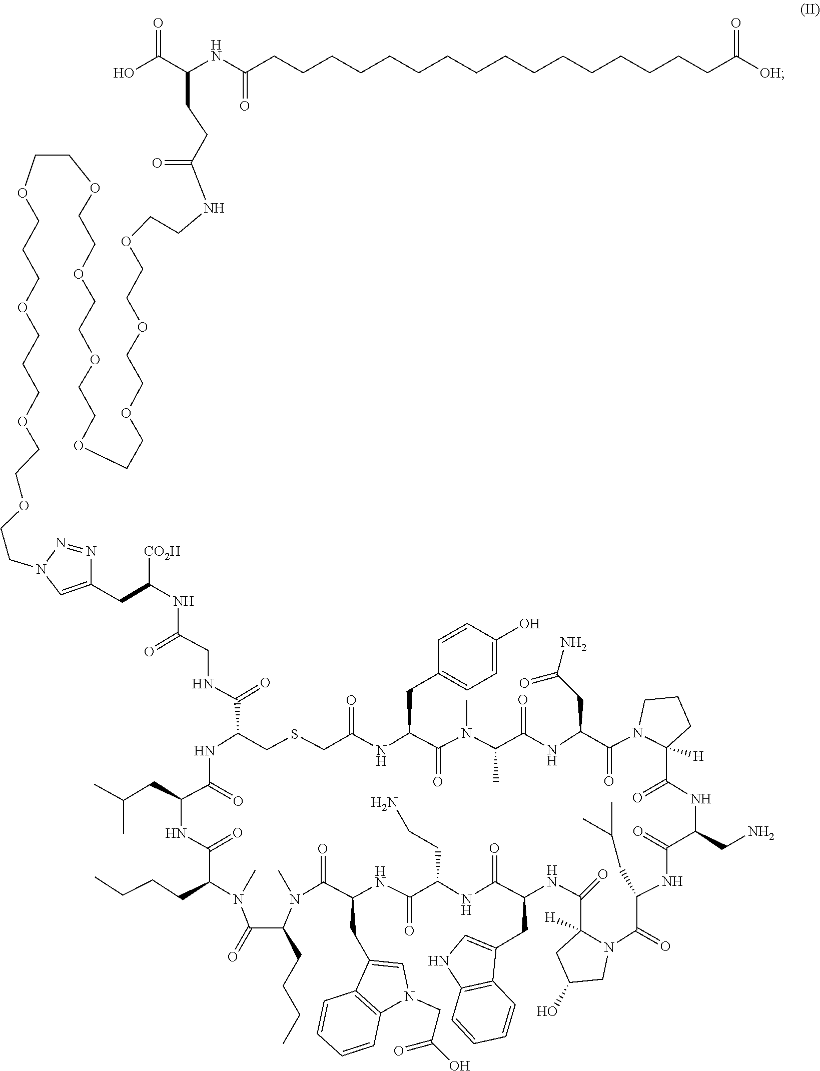 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof, to >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%, the method comprising formulating the biologically active compound with a salcaprozate salt and nicotinamide.

43. A method of inhibiting growth, proliferation, or metastasis of cancer cells in a subject in need thereof, said method comprising administering to the subject a therapeutically effective amount of a pharmaceutical composition of any one of claims 1 to 34 and 41.

44. The method of claim 43, wherein the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and a hematological malignancy.

45. A method of enhancing, stimulating, and/or increasing an immune response in a subject in need thereof, said method comprising administering to the subject a therapeutically effective amount of a pharmaceutical composition of any one of claims 1 to 34 and 41.

46. A method of blocking the interaction of PD-L1 with PD-1 and/or CD80 in a subject, said method comprising administering to the subject a therapeutically effective amount of a pharmaceutical composition of any one of claims 1 to 34 and 41.

47. The method of any one of claims 43 to 46, wherein the formulation is administered orally.

48. The method of any one of claims 43 to 46, wherein the formulation is intravenously administered.

49. The method of any one of claims 43 to 46, wherein the formulation is intraduodenally administered.

50. A method of administering a compound of formula (II): 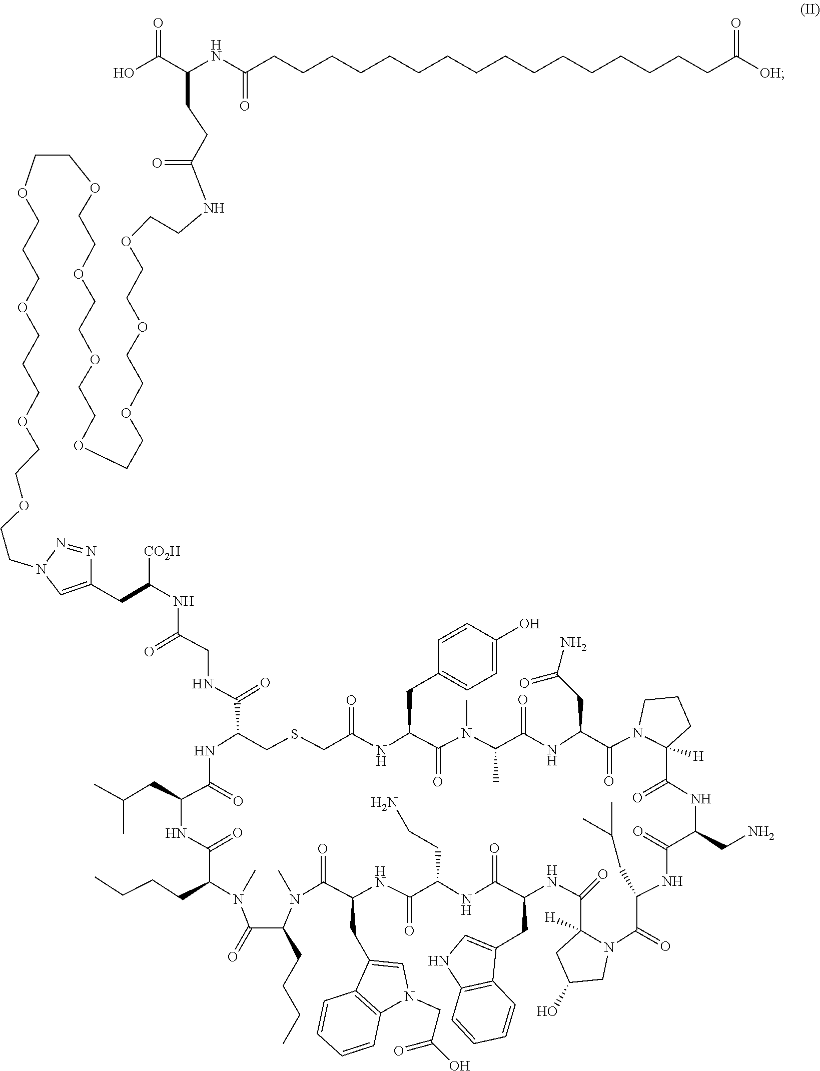 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
comprising administering the compound orally with salcaprozate sodium and nicotinamide wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

51. An orally administered composition comprising a compound of formula (II): 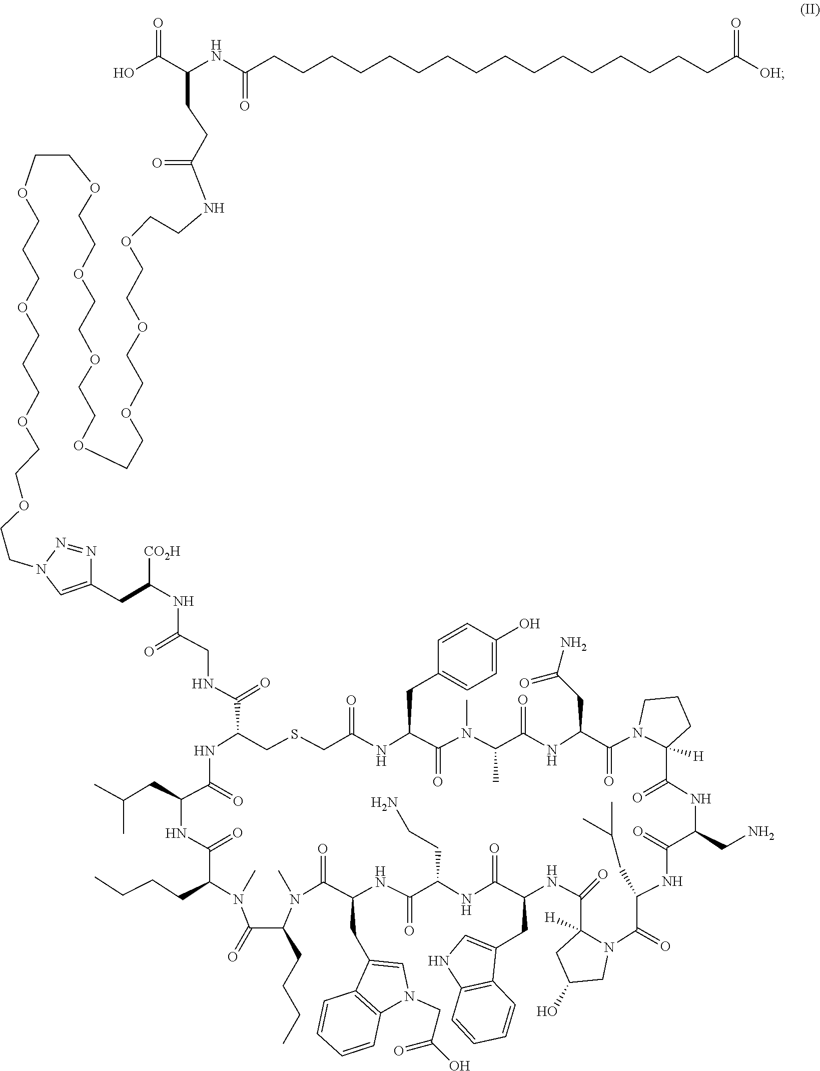 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof,
salcaprozate sodium, and nicotinamide, wherein the oral bioavailability of the compound of formula (II) is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

52. A method of administering a compound of formula (II): 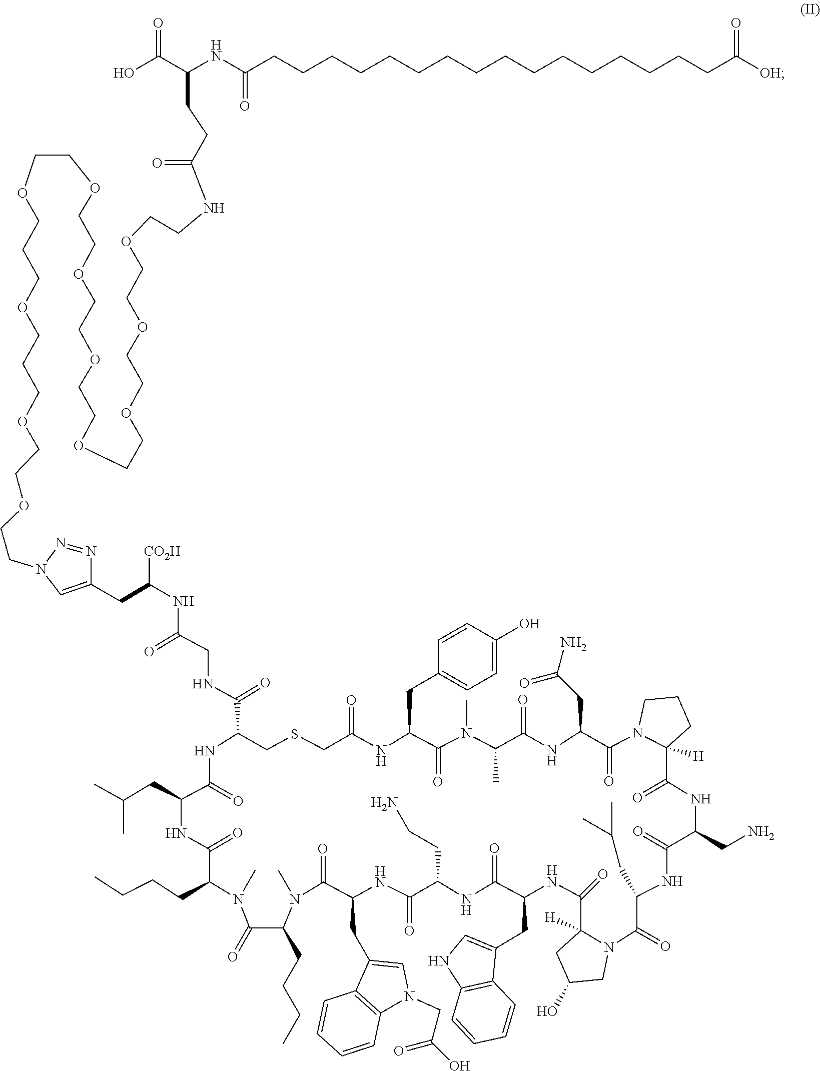 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
comprising administering the compound orally with salcaprozate sodium and nicotinamide wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.

53. A method of orally administering a peptide comprising orally administering a compound of formula (II): 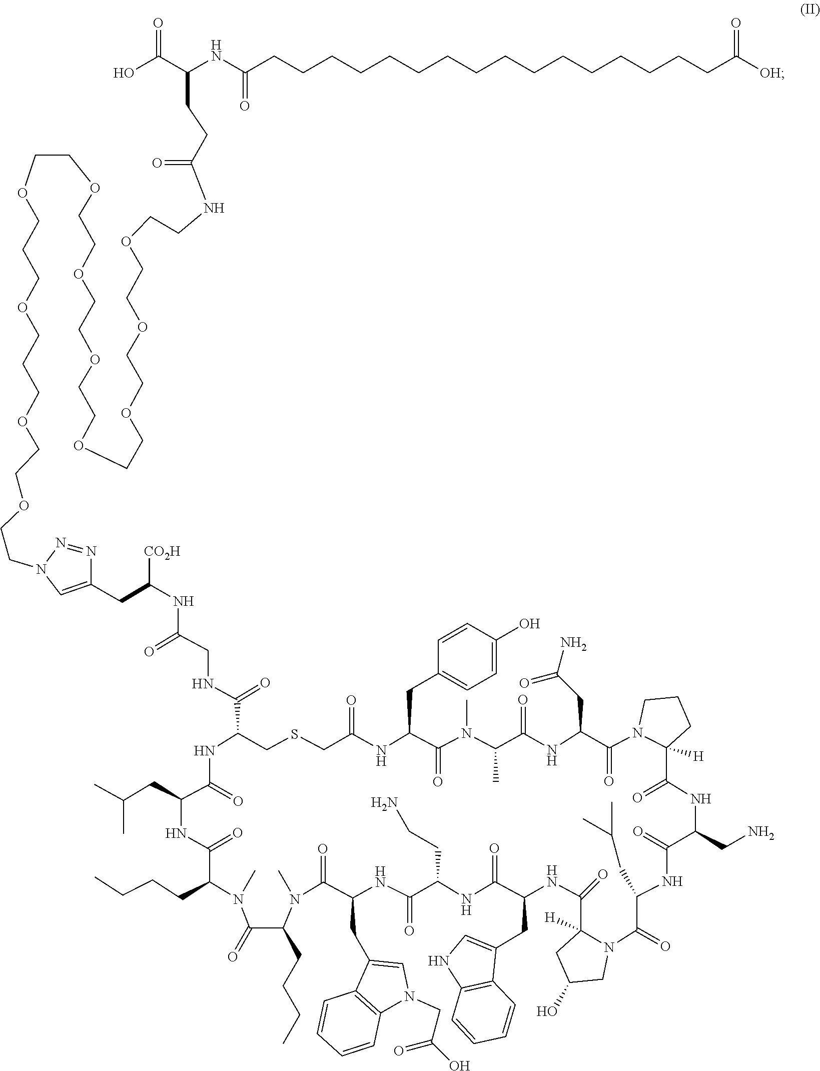 [SMILES: CCCC[C@H]1C(=O)N(C)[C@@H](CCCC)C(=O)N[C@@H](CC(C)C)C(=O)N[C@H](C(=O)NCC(=O)N[C@@H](Cc2cn(CCOCCOCCCOCCCOCCOCCOCCOCCOCCOCCOCCOCCNC(=O)CC[C@H](NC(=O)CCCCCCCCCCCCCCCCC(=O)O)C(=O)O)nn2)C(=O)O)CSCC(=O)N[C@@H](Cc2ccc(O)cc2)C(=O)N(C)[C@@H](C)C(=O)N[C@@H](CC(N)=O)C(=O)N2CCC[C@H]2C(=O)N[C@@H](CN)C(=O)N[C@@H](CC(C)C)C(=O)N2C[C@H](O)C[C@H]2C(=O)N[C@@H](Cc2c[nH]c3ccccc23)C(=O)N[C@@H](CCN)C(=O)N[C@@H](Cc2cn(CC(=O)O)c3ccccc23)C(=O)N1C]
or a pharmaceutically acceptable salt thereof,
salcaprozate sodium, and nicotinamide, wherein the oral bioavailability of the compound is >0.3%, >0.4%, >0.5%, >0.6%, >0.7%, >0.8%, >0.9%, >1.0%, >1.1%, >1.2%, >1.3%, >1.4%, >1.5%, >1.6%, >1.7%, >1.8%, >1.9%, >2.0%, >2.1%, >2.2%, >2.3%, >2.4%, or >2.5%.
